SEQUENCE LISTING (24 sequences)

SEQ ID NO: 1 - DNA, 18193 bp, Homo sapiens
  misc_feature (1)..(18193): Human CD22 genomic DNA Position 35820072 to position 35838264 of GenBank Accession No. NC_000019.9
acttctccttttgctctcagatgctgccagggtccctgaagagggaagacacgcggaaacaggtaaaaatcattttgcttttattttgcattcaacaagcaagttattacggaacagcagttatgggccaggcatacctcccagagctgggaacacagtggggacctccctggctctctcttaccggtgttacaacaggttgtagacagacccctgtcttgagcatcctccttgccaggcctgctgagtcttctgagagtagggtaggttattggatgcccaggagggaagaaggagccagggaggtcagccccaaggttctgcaaggccctcaacaggcctggactgaggaggtctggacagcatggccctgtcctgagcctctgtgcataataactgctgtccctaacctccaccccaccctcagccttccaattcccgggcctggggccctactcctgtgctccagagactcctggagctccttgaggcagcacacagtcctgctctggaggcgcccatctcccactcatgctgggatgctccagcccgtcccagagcaggttgtggctggagggtgctggcagaggagggacaatggcccggctcctggaggcaagtgttggctgcagggaacggagtctagtccttgccacagcccttgttaccccttaggtaaccttaaggggatttcaaagaactctggctctgcaaccctgctaagttttttatggaacatgtaaaatagatcccatggccaaagaagtatggacaatgtattatactatactctaatccccatgtctagagattaatggtgtagatagagtttactgaaaggtttttaaagtcctgcagtaaagaatcttacttaagccaggtgcggtggctcacgcctgtcatctcagcactttgggaggccaagttggggggatcacctgaggtcaggagtttgaaaccagcctggccaacatggtgaaaccccatctctactaaaacaaacaaacaaacaaacaaatcagacgggtgtggtggcacatgcctatagtcccagctactcaggaggctgaggcaggagaatcacttgaaccctggaggcagaggatgcagtgaactgagatcacaccactgcacttcagcctgggcgacagaagaatgagactctgtctcaaaagaaaaaaaaagaatcttacttaactcttttaccctgcctttctcaagtgtatgtgaccttgaagatgttttttgcatgtgacatgatgagtacctgcggggctgatgtcagctctgcctggggaatctgcctcagcttcagaccctgcaccccaaattcatgcttagcccaggccagacccccttcctgctgtcggcaagcagcttagctttccgcacacccatgaacagatacccagcagcctttggcatgagccatgtggcccgaggagataaacagacagagagaggaacattgatgcgtgtgaaatgttggcaggtgagtgggaggggcttgggttggtgctgagaccggagacacactctgctgtcacatccttcagggtccccgggggccagcacggagggccatgatcatgtaaattgggacttaaatttaactctatgtaaatataactctatgcagaggtcagggatggtagaggacaagctcactgcagagaaagaaagaaagatgggcaacagttgcagaacctaacccacagataggttttgtttgaccagctctgtacatttttagtttattttttatctttttgagacagggtcttgccctgtcgcccaggctggagtgcagttgcatgatctcggctcattccaacttctacttcccgggttcaagcaattctcctgcctcagcctcatgagtatctgggattacaggtgtacgccaccacgcctggctaattttttgcattttgagtaaagatggggttttgccatgttggccaggctggtcttgaactcctgacctcaagtgatctgcctgccttggcatcccaaagtattgggattacaggtgtgagccactgcgcctggctcacccatactttgttactggttttagaattcctttttacctggtctttcaaaccaagttttgaccatgtctgtccctgagtataatccttgatgagtagcaccagagagagctccacgttacccggggcctggcattcaaagcactcagtggtctggtgccactcaaagcctgtgagccaggcaggaagtttataaaattagagatggatgaatggatggatgggaaaaagcaagcaagggaggaaggaaggaagaaaggaaggagttctgggaagagagagaggctgcccccgcagggctctcagtttgaaggagaggtgcagcacactcagaaaaaactcccggccaggagtggtggctcacacctgtagtgccagcactttgggaggccaaggctggcagatcacctgagatcaggagttcaagaccagccaggccaacatggtgaaaccccatctctactgaaaatacaaaaatcagccaggcgtggtggcacctgcctttacccagctacttggggggctgaggccagagaaacgcttgagcctgggaagcagaggttgcagtgagcggagatcacaccactgcactctagcctgggagacagagcaagactctgtctcaaaaaaaaagaaagaactccgtgagcatcccaaatgccacatccccataatgcaaccagacccggtcctcccggatccaggggaagggtcggagctcagtgggtgagcaagaaaagccacgtggaagaagtaagcggccagatggctcaaagacaaactctcccctgctcaggcttgcacccagacacgacaccatgcatctcctcggcccctggctcctgctcctgggtaaggactgtggcctgggctagtactggggttctgggatgtcagtgggcactggaggaggaggaaagacccaggcagagaggcgtcaacatagggtagggtgggggcagcggtgtgtatagataaatgtacatacaaatatatatgtttcccatatatacatacacttctctcattgcatcctgctgtctttgtgtatctctgtatttctcccactatcttcagagggaattctttagtctttagaaagctctctgctgggtgcagtggctcatgcctgaaatctcagcactttgtgaggctgaggtgggaggatcacttgaggtcaggagttcaaggccagcttggacaacatagcaagacccctatctctaaaaagaataaaaaattaaacaaattttaaaataaaaaataacttttaaaagaaggctctcatgcccccttagtaatgctttctgatcactgtggtgagttctagaatacttggctttctctgactcaagtaaatgggtttttgagcaccctgaaaccctctacgcctgggagggggcctgcgtctggatcccctgcacctacagagccctagatggtgacctggaaagcttcatcctgttccacaatcctgagtataacaagaacacctcgaagtttgatgggacaagactctatgaaagcacaaaggatgggaaggttccttctgagcagaaaagggtgcaattcctgggagacaagaataagaactgcacactgagtatccacccggtgcacctcaatgacagtggtcagctggggctgaggatggagtccaagactgagaaatggatggaacgaatacacctcaatgtctctggtaaggccttcggggagcgggtcctctgctctgggcaggggtgagtgggaggcaggaaatacctgactcctggcacagagctcacaaaccgagcttcctgcagagctcaggcaggggacgccagcggatgacgatggcgatgcagcagcactagacagagctgcgggacctcggatgtcccatctgaccctcagttcctgcccctctgggacctggattccaaagttatcggatctcctgatctttccctctttctttctcaccatgtatttttcagcttttgtgacaaactcgtgtttggccttagtactggaaccatccaagcaccgcattcctttagtggggtgggtctgcatgtaaccagcctcagctctcccccaggctggaccgcccccttcagacattggggtctgaagctgcatcgctcaccccaaacttagggctgctcagagctccctatagaggaaactgggtgtggaacggagtaaccacactccctagggcagaggccccagacctgtgtttgatgggtcaaatccacccctacatgtgcagtgccataactgagcctgtgttataaaaatcataaaatgtttgttttactttttttcctcttcttttccccttccccttcctttccctttttatgagacagggtctcgcactgtcatcacccaggctggagtgcagtggcgcgatctcagctcactgcagccttgatctcccgggttcaagcaatcctcccacctccaaagtagctaggatcacaggtgcatgccaccatgcccagctaactttttaatttgttgtagaaatgggtcttgctatgttgccaggctggtcttgaactcctggcctcaagcaatcctcctgcttcaacctctcaaagcattaggattacaggcatgagtcaccgcacccggcctgatcttctgatttttcaagagacgatggaaatctagattttccagtgaaatctcccagcatgttaatgttcatggcaagttctcatgagctcttcaaggtcagatgacactgttgttgaaccccaaaggccaggtttgagctcccgatgcgtagtcagccaaacactgacacctcagtgcttagaagctgagataggattatttaatttagccaaagcaaggggacagaagagagaaactctcaaatctgacctgactttgagcgttagtgggggcttttatgagtaaagtaggtgtgcaggaggtgacctcccgatcgtcaaagctgttgtgcccctcggctcatcaaatttctggatgtcatcaaggaggtctgtgtgacctaaggatcattgttctttgaaagagagacaagttcatttatcttgcaggcagctgccagggagtcaggatgtaagttaaacaattatgagtgaattaatggttacctgctactgaaatgaccaaatgtaactaatcatgcatggaggaaggaaaggacaaagagaaaagaaaagaagtaaaacgaacatcttataatttttataacataggttcagttatagcactgcacatgtaggggtggctttgacccatcaaacacaggtgtggggggcccctgccacagggagtgtggtcactccgttccacaccgtttcccggatgggcagttctgagcagccttaaatttggggtgagcaatgcaacttcagaccccgctgtctgaaggaggtggtccagccttggggagagctgaggctgggtgcacacagacccactccactaaaggaatgcggtgcgtggatggctgcaatactgaaggaagccatgaaagtaacacaagtttgtcaaagctgaaaagtccgtgaactaaactgataaaattcacttagaggtggtgggaataggccgggcacggtggctcacgcctgtaatcccagaactttgggaggccgaggcaggcggatcacaaggtcaggagatcgagaccatcctgactaacacagtgaaaccctgtctctactaaaaatgcaaaaagttagccaggcgtggtggcaggcgcctgtagtcccagctactcaggaggctgaggcaggagaatggcgtgaacccggaaggtggagcttgcagtgagctgagatcacgccactacactccagtccgggcgacagagcgagactctgtctcaaaaaaaaaaaaaaaaaaaaaagaggtggtgaggtggtgggaatagaactttgcaatcattcagaaagcagacatccggcaatgagcatgaaagaaaagtagatcacagttcccaaggaggcttagtgtggtcaaggcctttcagaggggatttcgggtgagcaaaagtgattcaaagtggagaaaagcaggactgcgttgggtgcagtggttcccgcctgtaaccccagcactttgggaggctgagatcggaggatcgtttgagcccaggagtttgagaccagcctgggtaacatggcaagaccctgtctctacaaaatgtttaaaaaactagctaggtgtggtggcacatgcctgtagtcccagttactcagggggctgaggtgagaggattgcttgagcctaggaggttgaggctacagtgagctaggattgcgccactgcactccagcctgggtgacaaagcaagaccctgtctctaaagaaaaaaagaggccgggtgtggtggcttacacctgtaatcccagcactttgggaggccaaggcgggaggatcacttgaggtcaggagttcaagaccagcctggccaacatggcgaaaccctgtctctactagaaatacagaaaaattagctgggtgtggtggtgggcacctgtaatcccagctactcgggaggctgaggcaggaggatcacttgaactggggaggtagaggttacagcgagccaagatcgcgccactgcactccagcctgggtgacagagggaaactccatctcaaaaaaacaacaacacaacaacaacaacaataacaacaaaaaaacaaagcaggactggagagaggtggaatgaagtggcaaggggttcctgaggggtgatttgggacaggacatctaaagccaggtgtacgctcacgtcctcagtcccccaggctcctgcacgggctctgttcttttgcagaaaggccttttccacctcatatccagctccctccagaaattcaagagtcccaggaagtcactctgacctgcttgctgaatttctcctgctatgggtatccgatccaattgcagtggctcctagagggggttccaatgaggcaggctgctgtcacctcgacctccttgaccatcaagtctgtcttcacccggagcgagctcaagttctccccacagtggagtcaccatgggaagattgtgacctgccagcttcaggatgcagatgggaagttcctctccaatgacacggtgcagctgaacgtgaagcgtgagtctccccggcatgcctgtgggaagggcaaggtctgtgtcaccttctccccagccccgcagggggcatgcacccagggcagggggaagcctgcacagacggcggcatcctccagccctggtcacgccgccttgtcagccctggtgtttcgggaaaaagatttgctctagcctaacagaataaaatggtccatcctcaagccatgacatgaattggggattatctggttaggtctttttgttcccctcttggtggggatttttttcgcatcattatcttgtgcctcattcattcaataaatacgtatcatgaacctactaggtaccaggccttattacggctgccaatggggggcatggggcggtgggcagggtgcagcagtgagcaaaactcttgccccacgcggagccagcgctgcagtgaaagagacagacaacaaatggattaccaaagaaatacagagcatgagccaagacattagaatctggaacaaagcaatgttaacaaagaaatatacaacactattgtaggtagtgatatgtgtgttaggaaaaaaataaggccgagagaggggagtgatggagagagacctctctaagaaggtgagcacttaggccgggtgcggtggctcacgcctgtaatcctagcactttggaaggccgaggcggggggatcacaaggtcaggagatcgagaccatcctggctaacatggtgaaaccccatctctagtaaaaatacaaaaaattagccaggcatgatggcaggcgcctgtagtcccagctacttgggaggccaaggcaggagaatgacatgaacccaggaggcggagcttgcagtgagctgagatcgcaccactgcactccaacctgggtgacgagtgagactccatctcaaaaaaagaaaaaaaaagaaggtgagcacttaagctgactggaacggagggagtggctgaggccggcacatatttgggagaggaggatttcagggaaagagaagggcaggttcaaagctttgaggcagcagctgggtgggaggacctcagaggctgaagttcaccgcctctgagggccacatggtaggacaggaggcaggagatggagcaggactctggatcccgagggctgaggtgagggtttgggattctatgcaaagcacagaacccgctcgtggtttccagcaggattggcgaggtctggtagatggtgttaggaaccctgcgtgctgctggtggagaccaagaagtagaggaagtgggagggggaagctgagagccgagttaggaggctgtgaccatcacctgggtgaagggactggcaggccatggctttgtcagaataaaagcactttccacaccctccaccctctgaggattccccgccccctccccgactgcccctctgctcctccagacaccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaagtactgctgtcaggtctccaatgacgtgggcccgggaaggtcggaagaagtgttcctgcaagtgcagtgtgagcccctcggagctggggacaggccaggcagggaggtagcaggggtggacccggagaggggagccacgggggctctcggggccgtgtgcacaggttgggggtgctctcctcacccctccactcgcctctgccccctcttccagatgccccggaaccttccacggttcagatcctccactcaccggctgtggagggaagtcaagtcgagtttctttgcatgtcactggccaatcctcttccaacaaattacacgtggtaccacaatgggaaagaaatgcagggaaggacagaggagaaagtccacatcccaaagatcctcccctggcacgctgggacttattcctgtgtggcagaaaacattcttggtactggacagaggggcccgggagctgagctggatgtccagtgtgagtagccacggagcctctggttctagggagagaagatggacacagggaacggggaaggcagatggggtgcagggcattccggggtcctggagtcaagagcaaggagcagccagggtctcctgttcacaactgctgtctcagctcctagcatagggcctggctcatggagggtgcttggtagatgcttgtttttttgtgttttagttttttttgagatggagtctccctctgtctcccaggctggagtgcagtggcacaatctcggctcactgcaagctccaccttcccgggttcatgccattctcctgtctcagcctcctgagtagctgggatgacaggcgcccgccaccacgcccggctaattatttgtacttttagtagagacggggtttcaccgtgttagccaggatggtctcgatctcctgacctcgtgatccacccgcctcggcctcccaaagtgctgggattagaggcgtgagccaccgcacccggcctgtagatgcttgttgaatgaatgaggccaggtgcggtggctcccgcctgcaatcccagctctttaggaggctgaggcaggagggtctcttgaacccaggagtttgaaaccagcctgggccacacagcgagataccatctctacaaaaaaatacaaaaattagctgagcatggtggtgcgtgcctgtggtcccagctactggggaggctgaggagggagaatcacttgagcccaggaagtgaaggctgcagactgaactatgttcctgctactgcactctagcctgggtgacagagcaagacctgggtctcaaaaagaaaaagaaaatgctgggactgggattataggtgtgagctgggcacagtggctcacacttgtaatcccagcactttgggaggccaaggtaggcagctcacttgaggccacgagttcaagcccagcctgggcaacatggtaaaaccccatctctacaaaaaaaaattagcctggcatggtcatgcatgcctgtagtctcagctacccaggaggctgaggtgggaggatcacctgagcctggaagtagaggctgcagtgagcctgtgatcatgccgctgcactccagcctgggtgactgagtgagaccttgtctcaaaaaaaagaatggattggccaggcatggtggcttacgcctgtaatcccagcactttgggaggccaaggtgggcagatcacctgaggtcaggagttcgagaccagcctggccaacatggcgaaaccctgtctctactaaaaatacaaaaaattagccgggcatggtggcaggcacctgaaatccacctacctgggaggctgaggcaggagaatcgcttgaacccaggaggcagaggttgcaatgagctgagatcatgtcactgcactctagcctgggtagagtgagattctgtctccaaaaaaaaagaataattaactgcaagataccaggactccatgagggacacagagacttagcgtccctaggcagtggctgagtcttgagagatcttgaaatttttaatgagaactggggcaaactgtctcattacctctccattgtaagacccttttaacacagtgaatcttgttaccatgtagagcacaggacaaaaagaacacataagctcgatccttggaccctgagatgtcatatgtgctaacaggacaggggtgcccaaatcctgagcatctcagacagctggatgcgtgaccacagcagccacagtgatgtttgtggagtgtttgccacacgccacactctgctctaagggagtcatctcatttccttcttgaaacaattcccaggaggttaggaactctcaggactcaccccccacccaccccgcactgcctcttgtttttaagatggagtctccctctgttgcccaggctggagtgcagtggcttgatctcggctcactgcaacctccacctcccgggttcaagcgattctcttgcctcgaccacctgagtagctgggactacagacatgcaccagcatgcatagctaatttttaaaatttttagtagagacagggttttgctgtgttggccaggtctcgaactcctgacctcaggtgatctgcccacctcagcctcccaaagtgctgaaattacaagcgtgagccaccatgcccggcctcaggaccccatttgacagacgtgtaaactgagacctgcagcccatgcccgtgcgcctctcagcagccgtctggcttcagcgctgtgccctgaacccctgcaccaaaccacagccaggccagagagccagggcagggtttctgttctcacacagatgcaggaaggacgcacaagcccccctttgattaatttaggacctgtgcagatgcccgggacaggtagcgggagaagaggaagcaggagggaaggggtactgtggacagctggcttttctttactcacctctctggttttcttccagatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcagcttgtaatagttggtgctcgtgggcctcccctgtcgccctgaatgtccagtgtgagtccctgggctaggcagggggatctgggaggtggcccggctgggatgaggggatgaaggcaacgaggccggagcctgggccagtgtcttcaacagaattgagggacaggagcctggcgtcagggccaaggggaggggaggcctggggacagcaaaagggacagggagcggagaggtcagcttgggacccttgccctccagatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtgtgagtgagggccggaggctgggagtggagcagagaagggaccagtggcctgcctggtagtgacttcgcaccccctccccctgcccgccatgcagatgcacccaggaggctgcgtgtgtccatgagcccgggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactgtaaggcctcttcctgctcttgttcttcttggtggtggtcagtccttccttccttccttccttccttccttccttccttccttccttcctttctttctctctttcttttcctcctcctcttcttcccctcctcctcctcttcctccttcttcttcttcttctgctttctccctctcctcctcctctttcttctccttctccttccttcttcttcttcttatagagacaaggtcttactctgtcacccaggctggagtgcagtcgcataatgacacctcactgcagcctccaattcctgggctcaagcaatcctttcgcctcagcctcctgagtagctgggactatagacatgcaccaccacatccggctaatttttacatttttgtagagatggggtcttactatgttgaccaggctggtttcaaactcctgacctcaagtgacccttctgcctcagcttcccaaagccctgggattagagccatgagccactgtgcctctccctgtttcctctcatgctcctctctggccctgtctcctccagtgacttcccgaaaagccttccagaccagctggccccttcttccccacctcactgtcccctttcccattcacaccccttgctccaggccatctgccgctgtgctggaggagtcgtcttgtcctgcgtcaccagtctctctttttctacctcccctgcagtctctcctgcctccctctagggtcccctctcaggctgctccaccttccagaagtcctgagctcctactgggtccctgtctctctctctctgactactctgtctctctcagccctgacaggctccttgtttttatcttttaaaacgtaaatgattttttttttaaacggagtctcgatctgtcacccaggctggagtgcagtggcgtgatcacggctcactgcaagctctgcctcccgggttcacgccattctcctgcctcagctccccgagtagctgggactacaggcgcctgccaccatgcccggctaattttttgtatttttagtagagatgtggtttcaccgtggtctcagtctcctgacctcgtgatccgctcgcctcggccttccaaagtgctgggattacaggcgtgagccaccgcacccggctgattttttttttttttttttgagacagggtctcaccctgttgcacaggttggagtgcagtggcacaatctcagctcctgcagcctcgaccccctaggctcaagtgatactcctgcctcggcctccacagtagtaaacttgagtggtttttaaacagatcatacaagcacctggtaaaaaaataaaatgtaagcatgaaagggtaaatagtggaaagacagacgcggctcactcttgatccctgagaactctcatccccaccaaaggtcctcactgcacacagtcttcccatctcactgcacacagtcttcccatctcctccaggggtatttttcacacgcacaattcacacacagaggtctgtgcctatatacccttcctcatgacacagacgaggcagagcccacactttgcctgggaattttctttgcttttttccatgtaataatactgtttggacattgttctgtatctatgcatacacagagtgtctaaagtctggaggctgggcatggtggttcatgcctgtaatcccaacactttgagaggccatggtgggaggatcacttgagcccaggagtttgagaccagcctgggcaacatagcccacacctcatagcaggatctcatctctacaaaaaataccaaaaaaaattttaaaaacgaacaaacaaaacaataacaacaacaaaaaagagtctgaaagcatatggaagatacctaattaatttattgtactttttcagattaataattggaaacatggccttaaattgagggaacctgaccaggcacagtggctcacgcctgtaatcccagcactttgggaggccaaagtgggtggccacaagcactttgggaggccacaaggtgggtggatcacttgaggccaggagttccagaccagcctggccaatggtgaaaccccatctctactaaaaatacaaaaattagctgggtgtggtggcgcgcacctgtagtcccaactactcgggaggctgaggcaggagaatcgcttgaacccgggaggcagaggttgcagtgagccaagatctcaccactgcactccagcctgggtgaaagagcgagactctgtctcaatcaatcaatcagtcaatctagggaaccatcaccagcacggccgaaggccaaaggaaaacatctgaaacatgttatctggaaatgtaacagacacatcccatcatataaaaccattgttccctgggtatcccctttgcagacagcctgagagcctcctctttttcctggggccatggagtcctcgcgtatgaaggttgcatacatcctcctcccagtgccctcttcagggcctaggtctgcagcctcttgttttgggcacgcctgcaaggacagttgcaagatgcattcccatgtggaattgctgggtggaagacaggtgcactttcagagctgatcacacagctatactgccgtgaagagctgcacccatttatgcgggtgcgggtcacaggctctgggcgtgccacgtctgtgggaaaagcagctcctcaggagaattagtgccttattcctaaagggaggcccagagcaagcaggggccgttgtcaccctcttcctctgcacccctgcacgcatgcaggggagaaggacgagtctggctgcagagagaagtccaggatgccttccaggcaaaggctgccagggagagctggcccctcagttgattaaggtctctcctttctccacccagatagcccggagaccatcggcaggcgagtggctgtgggactcgggtcctgcctcgccatcctcatcctggcaatctgtgggctcaagctccagcgacggtgagctcctgccatcccccaccacctcctctatcccttggcagaggcccgtgtccagttgctccatctcgaagcctccagcctgtggagtccctgaccctcacacatgtgcctttatttctcagttggaagaggacacagagccagcaggggcttcaggagaattccagcggccagagcttctttgtgaggaataaaaaggtaggatggggctgggcacgatggctcatgcctgtaatcccagcactttgggaggctgaggcaggtggatcacctgaggtcaggagttcgagaccagcgtagccaacatggtgaaaccctgtctctacaaaaaatattttaaaaattagccgggcgtgatggtccatgcctgtaattccagctactcgggaggctgaggcaggagaatcgcttgaacctgggaggcagaggttgcagtgagccaagattgcgccattgcactccagcctgggtgacatactgagactctgcctcaaaaaaaaaaaaaaaaaaaaaaaaagggtaggcatgaggcagactgtgaagctgagtggggaaacaaggtgaaggaaggggataaaatgtcctctgaggagacctgggctgtcagaggccagggaaggggacgagggttggggaacaggtggttagcacttcatcctcgtctccctcccaggttagaagggcccccctctctgaaggcccccactccctgggatgctacaatccaatgatggaagatggcattagctacaccaccctgcgctttcccgagatgaacataccacgaactgggtactgagggtaccaggagggtgaccctgcaccctgggagggaggcgggaggaaaagctctgtcccgcctgccctctttgtgccaggcccatgccaggcaccctgatacatgctctgcctcattcccactcggcaacaagcctctgggtaagggccagtctccagaagtggacctgctccttcaagtcagcagcacacatgcccagtccgttcttatcacaaagatcaagacctcatgatacacacccacgcctactccctccacgcacatttgcacgagctcctactgtgagctttgggcactggggaggccacaggttttacataaggggctgaggttggggtgctgttgggggctctgggtgttgagagggaggagagttcgtgggagatgcttcatgcgtggtcgtctatctgccctgtctctcagagatgcagagtcctcagagatgcagagacctcccccggactgcgatgacacggtcacttattcagcattgcacaagcgccaagtggtaaggagggtctccccaggtctccccagaggggctgtggaaggctggggacagggcctggcctcagtggtgggtcccacataggaaggagttgggtaggcatccgtggtggcagaggttgggtgttggagacggttgctccggcagagctggccagagccaggcaggtacctccaggtcctggatgccggccacagccagtttcctgacacgaggacacccgcctgggcttttggacccccgggtggaatgaaggagagaatgcggagaaaagcggcggtggaggctggcgacagtggggccaggctaaccaccatgcggttttctcagggcgactatgagaacgtcattccagattttccagaagatgaggggattcattactcagagctgatccagtttggggtcggggagcggcctcaggcacaagaaaatgtggactatgtgatcctcaaacattgacactggatgggctgcagcagaggcactgggggcagcgggggccagggaagtccccgagtttccccagacaccgccacatggcttcctcctgcgcgcatgtgcgcacacacacacacacacgcacacacacacacacacactcactgcggagaaccttgtgcctggctcagagccagtctttttggtgagggtaaccccaaacctccaaaactcctgcccctgttctcttccactctccttgctacccagaaatccatctaaatacctgccctgacatgcacacctccccctgcccccaccacggccactggccatctccacccccagctgcttgtgtccctcctgggatctgctcgtcatcatttttccttcccttctccatctctctggccctctacccctgatctgacatccccactcacgaatattatgcccagtttctgcctctgagggaaagcccagaaaaggacagaaacgaagtagaaaggggcccagtcctggcctggcttctcctttggaagtgaggcattgcacagggagacgtacgtatcagcggccccttgactctggggactccgggtttgagatggacacactggtgtggattaacctgccagggagacagagctcacaataaaaatggctcagatgccacttcaaagaa

SEQ ID NO: 2 - protein, 751 aa, Homo sapiens
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKRCRVLRDAETSPGLR

SEQ ID NO: 3 - protein, 15 aa, Homo sapiens
RCRVLRDAETSPGLR

SEQ ID NO: 4 - DNA, 24 bp, Artificial Sequence
  Therapeutic Morpholino
gactctgcatctctttttattcct

SEQ ID NO: 5 - DNA, 20 bp, Artificial Sequence
  Primer
gcccgggggaccaagtgatg

SEQ ID NO: 6 - DNA, 25 bp, Artificial Sequence
  Primer
gtggaagagaacaggggcaggaagt

SEQ ID NO: 7 - DNA, 21 bp, Artificial Sequence
  Probe
cctgcctcgccatcctcatcc

SEQ ID NO: 8 - DNA, 38091 bp, Homo sapiens
gatcatgcctttggtgtcatgtctaagaacacttcatcaagctctaagtctcacagattttctcctatgttatcttccagaggttttttgtttttattttattttactttttaattaagacagagtttcgctctgttgcccaggctggagtgcagtggcaggatctcagctcactgcaacctccgcctcccaggttcaagtgatcctcctgccttggcctcctgagtagctgggactacaggcacgcaccacccaactggctaattttttgcatttttatgtggttgctttcgagaattttctctgcctcttgtttttagaagtttgactatgatgtgtcttggcatgaatttctttgggtttatcctgtttgtgatgcgctcagcttcttgaatctgtagacatatgttgtgcgtgtgtgttgtcaaatgaagataatttgcagccattacttcttggaatatttttttaaggcccaccctccttttcctccccttctgggacttcaatgacaaaatgttagatcttcttttattatagttactcatgtccttgaggctttgttccattttctttcatctattttctttccattgttcagaatgggtaatttctattgttatacgttttgtttcactgcttctttcttctatactctccattctgctattgagcccatccattatggtttttatttcagctcttgtattttttagttgtattttttagttctaaagtttccatttggttcttctttatattttctatttctttgctgatactttttattattttatttgtttcaagcacatttataatttctcagtgaaacacttttatgatggctgctccaaaatctttgtcagataattctaacatctgtgtcgtcttgatgtttgtgtctgttaattatcttttgttattcagtttgagaatgcctgattcctagtatgacaagtgatttgccattgaagtcaggatattctgggcactgtgttataagattctggatcttctttaaatattttgttttagtgggcttccctttatactgctccagcgaaataaagtagggcactgcctgattgcttccagacagggataaaagtccaggttctccatccagcctttcctgaacctggggggttgggagagatgaggcctccatgttattgtttggtaagggtagaagttcaggttccccactaggaatccatgacaactccctggctgggagtgggagggatgcttcattactgctccctatgtggctttcactgacactgtgtgtggcagagagaggggggtgcttgttaccactgagtggtggcgaaagtcccaactctccaccaggccccctttgacactactccaatggggccgggtggaatgcctcatttctgcattggggtggcagtggatgtccaggctgcccacatggtttccaccaacaccacatcgggggagggtgatcccagtggggaggaatgtcccagctccaactctagcatcactctggctgagtatgggagtggcctagggttctttgttacagcctggtgaaggtgaatgtctaggttttccatccggcctttgctgacaagggtgatgatggaacttggatggagcggagcaggtatggactaaaagagctctgtctttctaggatgcccctttcctcgttctttggctagaaaagcatagttttgctagaacttgttctgttgggactttttcttgtttgtgccccttggcattttcagttgttggcttctccagtattcagcctgagatatatgaggcaaagagaaatcccagggaattcaccaccattccttaagtcccgaggttcctaaccagtctgcttctctccatctttcagtcttcttatgcttgtcttgtcacgttcaaggttttcagttatacttggcagaataaataaatacatctactccatctttccagaacatcactgctttttgttcattttatttatttattatttactgagacaaggtcttactttgttgcctgggctggagtgcagtggtgctatcatggctgactgaagccttgaactcccaggctcaagcgatcctcctgcctcagcctcccaagtagtttggaccacaggcatgggccaccatgcccagctacttttcattttttgcttttgttgactagccccacagagttttgctgtgttgttcaggttggtctcaaacttctggccttaagcaatcctcttgccttgctctcccaaagcgtattacaggcataagtcaccacacccagccctatcggtgcttttggaagaaggtgttcttccctctctctgtttccctccctatggccagctgcccatagagggcagtggtgctggagtgaagacctctcagcgcatccctcttcatctgtaggatgtgcatggcttccaatgctgtctgttctgctcctgagacactcgctggctacaggattataaaggtgatgaggtttcagcctaaagactctccattcaataacacttcctacaagatatcttttttgtttttttgagacaggatctggctctgtcacccaggctggagtgccacagcgcgatctgctcactgcaacctcagcctctcgggcttaagtgattctcctgccttagcctcctgagtagctgggaccacaggcgcccgccaccacacccagctaatttttggattttttttttttttttttttagtagagatggggttttaccatgttgcccaagctggtcttgaactcctgagctcaggcaatccacccgccttggccacccaaagcgctaggattacaggcatgagccaccatgcccagtcctacaagatatcttttctttttctttctttctttctttctttctttttttttttttgagacagagtctcactctgtcacccaggctggactgcagtggtgtgatctcttctcactgcagcctccgcctcctgggctcaagtgattctcctgcctcagccccctgagtagctgggactacaggcgcctgccaaaacacttagctaattttttgtatttttagtagagacggaatttcaccatgttcgccaggctggtctcgaattcctgacctcaagtgatccacccgcctcagcctcccaaagtgttgggattccaggcatgagccaccgcacccggccgagatatctttttcctcctcaatatctatgtgctcctccctcaacccttgggggcttcagtgtaggtggtcagattccccaccatttatgcctatgatgaaaagagtggaaatggtaataactggatggtgtcactaaactacaaatactttcaggtaggcctgtgcctttttcagcgggctgcagttctcctgcttggcttgagtcattcgcatttcctgagagctgggtagagggggagttgtggaggagcccattctgaaactgatctgatattgcaaacccatacacggaaaggaagaactgcccatacgtattcgagtcttccaatctgactccgagcccgcatccccctcaatcgtctcttttccccactccccagatcacggtgctgcttggtccccctcagagccatagagaagcagggggtgtggccatggaggggaaacctctgtcaccagagactttactgtacttctccttttgctctcagatgctgccagggtccctgaagagggaagacacgcggaaacaggtaaaaatcattttgcttttattttgcattcaacaagcaagttattacggaacagcagttatgggccaggcatacctcccagagctgggaacacagtggggacctccctggctctctcttaccggtgttacaacaggttgtagacagacccctgtcttgagcatcctccttgccaggcctgctgagtcttctgagagtagggtaggttattggatgcccaggagggaagaaggagccagggaggtcagccccaaggttctgcaaggccctcaacaggcctggactgaggaggtctggacagcatggccctgtcctgagcctctgtgcataataactgctgtccctaacctccaccccaccctcagccttccaattcccgggcctggggccctactcctgtgctccagagactcctggagctccttgaggcagcacacagtcctgctctggaggcgcccatctcccactcatgctgggatgctccagcccgtcccagagcaggttgtggctggagggtgctggcagaggagggacaatggcccggctcctggaggcaagtgttggctgcagggaacggagtctagtccttgccacagcccttgttaccccttaggtaaccttaaggggatttcaaagaactctggctctgcaaccctgctaagttttttatggaacatgtaaaatagatcccatggccaaagaagtatggacaatgtattatactatactctaatccccatgtctagagattaatggtgtagatagagtttactgaaaggtttttaaagtcctgcagtaaagaatcttacttaagccaggtgcggtggctcacgcctgtcatctcagcactttgggaggccaagttggggggatcacctgaggtcaggagtttgaaaccagcctggccaacatggtgaaaccccatctctactaaaacaaacaaacaaacaaacaaatcagacgggtgtggtggcacatgcctatagtcccagctactcaggaggctgaggcaggagaatcacttgaaccctggaggcagaggatgcagtgaactgagatcacaccactgcacttcagcctgggcgacagaagaatgagactctgtctcaaaagaaaaaaaaagaatcttacttaactcttttaccctgcctttctcaagtgtatgtgaccttgaagatgttttttgcatgtgacatgatgagtacctgcggggctgatgtcagctctgcctggggaatctgcctcagcttcagaccctgcaccccaaattcatgcttagcccaggccagacccccttcctgctgtcggcaagcagcttagctttccgcacacccatgaacagatacccagcagcctttggcatgagccatgtggcccgaggagataaacagacagagagaggaacattgatgcgtgtgaaatgttggcaggtgagtgggaggggcttgggttggtgctgagaccggagacacactctgctgtcacatccttcagggtccccgggggccagcacggagggccatgatcatgtaaattgggacttaaatttaactctatgtaaatataactctatgcagaggtcagggatggtagaggacaagctcactgcagagaaagaaagaaagatgggcaacagttgcagaacctaacccacagataggttttgtttgaccagctctgtacatttttagtttattttttatctttttgagacagggtcttgccctgtcgcccaggctggagtgcagttgcatgatctcggctcattccaacttctacttcccgggttcaagcaattctcctgcctcagcctcatgagtatctgggattacaggtgtacgccaccacgcctggctaattttttgcattttgagtaaagatggggttttgccatgttggccaggctggtcttgaactcctgacctcaagtgatctgcctgccttggcatcccaaagtattgggattacaggtgtgagccactgcgcctggctcacccatactttgttactggttttagaattcctttttacctggtctttcaaaccaagttttgaccatgtctgtccctgagtataatccttgatgagtagcaccagagagagctccacgttacccggggcctggcattcaaagcactcagtggtctggtgccactcaaagcctgtgagccaggcaggaagtttataaaattagagatggatgaatggatggatgggaaaaagcaagcaagggaggaaggaaggaagaaaggaaggagttctgggaagagagagaggctgcccccgcagggctctcagtttgaaggagaggtgcagcacactcagaaaaaactcccggccaggagtggtggctcacacctgtagtgccagcactttgggaggccaaggctggcagatcacctgagatcaggagttcaagaccagccaggccaacatggtgaaaccccatctctactgaaaatacaaaaatcagccaggcgtggtggcacctgcctttacccagctacttggggggctgaggccagagaaacgcttgagcctgggaagcagaggttgcagtgagcggagatcacaccactgcactctagcctgggagacagagcaagactctgtctcaaaaaaaaagaaagaactccgtgagcatcccaaatgccacatccccataatgcaaccagacccggtcctcccggatccaggggaagggtcggagctcagtgggtgagcaagaaaagccacgtggaagaagtaagcggccagatggctcaaagacaaactctcccctgctcaggcttgcacccagacacgacaccatgcatctcctcggcccctggctcctgctcctgggtaaggactgtggcctgggctagtactggggttctgggatgtcagtgggcactggaggaggaggaaagacccaggcagagaggcgtcaacatagggtagggtgggggcagcggtgtgtatagataaatgtacatacaaatatatatgtttcccatatatacatacacttctctcattgcatcctgctgtctttgtgtatctctgtatttctcccactatcttcagagggaattctttagtctttagaaagctctctgctgggtgcagtggctcatgcctgaaatctcagcactttgtgaggctgaggtgggaggatcacttgaggtcaggagttcaaggccagcttggacaacatagcaagacccctatctctaaaaagaataaaaaattaaacaaattttaaaataaaaaataacttttaaaagaaggctctcatgcccccttagtaatgctttctgatcactgtggtgagttctagaatacttggctttctctgactcaagtaaatgggtttttgagcaccctgaaaccctctacgcctgggagggggcctgcgtctggatcccctgcacctacagagccctagatggtgacctggaaagcttcatcctgttccacaatcctgagtataacaagaacacctcgaagtttgatgggacaagactctatgaaagcacaaaggatgggaaggttccttctgagcagaaaagggtgcaattcctgggagacaagaataagaactgcacactgagtatccacccggtgcacctcaatgacagtggtcagctggggctgaggatggagtccaagactgagaaatggatggaacgaatacacctcaatgtctctggtaaggccttcggggagcgggtcctctgctctgggcaggggtgagtgggaggcaggaaatacctgactcctggcacagagctcacaaaccgagcttcctgcagagctcaggcaggggacgccagcggatgacgatggcgatgcagcagcactagacagagctgcgggacctcggatgtcccatctgaccctcagttcctgcccctctgggacctggattccaaagttatcggatctcctgatctttccctctttctttctcaccatgtatttttcagcttttgtgacaaactcgtgtttggccttagtactggaaccatccaagcaccgcattcctttagtggggtgggtctgcatgtaaccagcctcagctctcccccaggctggaccgcccccttcagacattggggtctgaagctgcatcgctcaccccaaacttagggctgctcagagctccctatagaggaaactgggtgtggaacggagtaaccacactccctagggcagaggccccagacctgtgtttgatgggtcaaatccacccctacatgtgcagtgccataactgagcctgtgttataaaaatcataaaatgtttgttttactttttttcctcttcttttccccttccccttcctttccctttttatgagacagggtctcgcactgtcatcacccaggctggagtgcagtggcgcgatctcagctcactgcagccttgatctcccgggttcaagcaatcctcccacctccaaagtagctaggatcacaggtgcatgccaccatgcccagctaactttttaatttgttgtagaaatgggtcttgctatgttgccaggctggtcttgaactcctggcctcaagcaatcctcctgcttcaacctctcaaagcattaggattacaggcatgagtcaccgcacccggcctgatcttctgatttttcaagagacgatggaaatctagattttccagtgaaatctcccagcatgttaatgttcatggcaagttctcatgagctcttcaaggtcagatgacactgttgttgaaccccaaaggccaggtttgagctcccgatgcgtagtcagccaaacactgacacctcagtgcttagaagctgagataggattatttaatttagccaaagcaaggggacagaagagagaaactctcaaatctgacctgactttgagcgttagtgggggcttttatgagtaaagtaggtgtgcaggaggtgacctcccgatcgtcaaagctgttgtgcccctcggctcatcaaatttctggatgtcatcaaggaggtctgtgtgacctaaggatcattgttctttgaaagagagacaagttcatttatcttgcaggcagctgccagggagtcaggatgtaagttaaacaattatgagtgaattaatggttacctgctactgaaatgaccaaatgtaactaatcatgcatggaggaaggaaaggacaaagagaaaagaaaagaagtaaaacgaacatcttataatttttataacataggttcagttatagcactgcacatgtaggggtggctttgacccatcaaacacaggtgtggggggcccctgccacagggagtgtggtcactccgttccacaccgtttcccggatgggcagttctgagcagccttaaatttggggtgagcaatgcaacttcagaccccgctgtctgaaggaggtggtccagccttggggagagctgaggctgggtgcacacagacccactccactaaaggaatgcggtgcgtggatggctgcaatactgaaggaagccatgaaagtaacacaagtttgtcaaagctgaaaagtccgtgaactaaactgataaaattcacttagaggtggtgggaataggccgggcacggtggctcacgcctgtaatcccagaactttgggaggccgaggcaggcggatcacaaggtcaggagatcgagaccatcctgactaacacagtgaaaccctgtctctactaaaaatgcaaaaagttagccaggcgtggtggcaggcgcctgtagtcccagctactcaggaggctgaggcaggagaatggcgtgaacccggaaggtggagcttgcagtgagctgagatcacgccactacactccagtccgggcgacagagcgagactctgtctcaaaaaaaaaaaaaaaaaaaaaagaggtggtgaggtggtgggaatagaactttgcaatcattcagaaagcagacatccggcaatgagcatgaaagaaaagtagatcacagttcccaaggaggcttagtgtggtcaaggcctttcagaggggatttcgggtgagcaaaagtgattcaaagtggagaaaagcaggactgcgttgggtgcagtggttcccgcctgtaaccccagcactttgggaggctgagatcggaggatcgtttgagcccaggagtttgagaccagcctgggtaacatggcaagaccctgtctctacaaaatgtttaaaaaactagctaggtgtggtggcacatgcctgtagtcccagttactcagggggctgaggtgagaggattgcttgagcctaggaggttgaggctacagtgagctaggattgcgccactgcactccagcctgggtgacaaagcaagaccctgtctctaaagaaaaaaagaggccgggtgtggtggcttacacctgtaatcccagcactttgggaggccaaggcgggaggatcacttgaggtcaggagttcaagaccagcctggccaacatggcgaaaccctgtctctactagaaatacagaaaaattagctgggtgtggtggtgggcacctgtaatcccagctactcgggaggctgaggcaggaggatcacttgaactggggaggtagaggttacagcgagccaagatcgcgccactgcactccagcctgggtgacagagggaaactccatctcaaaaaaacaacaacacaacaacaacaacaataacaacaaaaaaacaaagcaggactggagagaggtggaatgaagtggcaaggggttcctgaggggtgatttgggacaggacatctaaagccaggtgtacgctcacgtcctcagtcccccaggctcctgcacgggctctgttcttttgcagaaaggccttttccacctcatatccagctccctccagaaattcaagagtcccaggaagtcactctgacctgcttgctgaatttctcctgctatgggtatccgatccaattgcagtggctcctagagggggttccaatgaggcaggctgctgtcacctcgacctccttgaccatcaagtctgtcttcacccggagcgagctcaagttctccccacagtggagtcaccatgggaagattgtgacctgccagcttcaggatgcagatgggaagttcctctccaatgacacggtgcagctgaacgtgaagcgtgagtctccccggcatgcctgtgggaagggcaaggtctgtgtcaccttctccccagccccgcagggggcatgcacccagggcagggggaagcctgcacagacggcggcatcctccagccctggtcacgccgccttgtcagccctggtgtttcgggaaaaagatttgctctagcctaacagaataaaatggtccatcctcaagccatgacatgaattggggattatctggttaggtctttttgttcccctcttggtggggatttttttcgcatcattatcttgtgcctcattcattcaataaatacgtatcatgaacctactaggtaccaggccttattacggctgccaatggggggcatggggcggtgggcagggtgcagcagtgagcaaaactcttgccccacgcggagccagcgctgcagtgaaagagacagacaacaaatggattaccaaagaaatacagagcatgagccaagacattagaatctggaacaaagcaatgttaacaaagaaatatacaacactattgtaggtagtgatatgtgtgttaggaaaaaaataaggccgagagaggggagtgatggagagagacctctctaagaaggtgagcacttaggccgggtgcggtggctcacgcctgtaatcctagcactttggaaggccgaggcggggggatcacaaggtcaggagatcgagaccatcctggctaacatggtgaaaccccatctctagtaaaaatacaaaaaattagccaggcatgatggcaggcgcctgtagtcccagctacttgggaggccaaggcaggagaatgacatgaacccaggaggcggagcttgcagtgagctgagatcgcaccactgcactccaacctgggtgacgagtgagactccatctcaaaaaaagaaaaaaaaagaaggtgagcacttaagctgactggaacggagggagtggctgaggccggcacatatttgggagaggaggatttcagggaaagagaagggcaggttcaaagctttgaggcagcagctgggtgggaggacctcagaggctgaagttcaccgcctctgagggccacatggtaggacaggaggcaggagatggagcaggactctggatcccgagggctgaggtgagggtttgggattctatgcaaagcacagaacccgctcgtggtttccagcaggattggcgaggtctggtagatggtgttaggaaccctgcgtgctgctggtggagaccaagaagtagaggaagtgggagggggaagctgagagccgagttaggaggctgtgaccatcacctgggtgaagggactggcaggccatggctttgtcagaataaaagcactttccacaccctccaccctctgaggattccccgccccctccccgactgcccctctgctcctccagacaccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaagtactgctgtcaggtctccaatgacgtgggcccgggaaggtcggaagaagtgttcctgcaagtgcagtgtgagcccctcggagctggggacaggccaggcagggaggtagcaggggtggacccggagaggggagccacgggggctctcggggccgtgtgcacaggttgggggtgctctcctcacccctccactcgcctctgccccctcttccagatgccccggaaccttccacggttcagatcctccactcaccggctgtggagggaagtcaagtcgagtttctttgcatgtcactggccaatcctcttccaacaaattacacgtggtaccacaatgggaaagaaatgcagggaaggacagaggagaaagtccacatcccaaagatcctcccctggcacgctgggacttattcctgtgtggcagaaaacattcttggtactggacagaggggcccgggagctgagctggatgtccagtgtgagtagccacggagcctctggttctagggagagaagatggacacagggaacggggaaggcagatggggtgcagggcattccggggtcctggagtcaagagcaaggagcagccagggtctcctgttcacaactgctgtctcagctcctagcatagggcctggctcatggagggtgcttggtagatgcttgtttttttgtgttttagttttttttgagatggagtctccctctgtctcccaggctggagtgcagtggcacaatctcggctcactgcaagctccaccttcccgggttcatgccattctcctgtctcagcctcctgagtagctgggatgacaggcgcccgccaccacgcccggctaattatttgtacttttagtagagacggggtttcaccgtgttagccaggatggtctcgatctcctgacctcgtgatccacccgcctcggcctcccaaagtgctgggattagaggcgtgagccaccgcacccggcctgtagatgcttgttgaatgaatgaggccaggtgcggtggctcccgcctgcaatcccagctctttaggaggctgaggcaggagggtctcttgaacccaggagtttgaaaccagcctgggccacacagcgagataccatctctacaaaaaaatacaaaaattagctgagcatggtggtgcgtgcctgtggtcccagctactggggaggctgaggagggagaatcacttgagcccaggaagtgaaggctgcagactgaactatgttcctgctactgcactctagcctgggtgacagagcaagacctgggtctcaaaaagaaaaagaaaatgctgggactgggattataggtgtgagctgggcacagtggctcacacttgtaatcccagcactttgggaggccaaggtaggcagctcacttgaggccacgagttcaagcccagcctgggcaacatggtaaaaccccatctctacaaaaaaaaattagcctggcatggtcatgcatgcctgtagtctcagctacccaggaggctgaggtgggaggatcacctgagcctggaagtagaggctgcagtgagcctgtgatcatgccgctgcactccagcctgggtgactgagtgagaccttgtctcaaaaaaaagaatggattggccaggcatggtggcttacgcctgtaatcccagcactttgggaggccaaggtgggcagatcacctgaggtcaggagttcgagaccagcctggccaacatggcgaaaccctgtctctactaaaaatacaaaaaattagccgggcatggtggcaggcacctgaaatccacctacctgggaggctgaggcaggagaatcgcttgaacccaggaggcagaggttgcaatgagctgagatcatgtcactgcactctagcctgggtagagtgagattctgtctccaaaaaaaaagaataattaactgcaagataccaggactccatgagggacacagagacttagcgtccctaggcagtggctgagtcttgagagatcttgaaatttttaatgagaactggggcaaactgtctcattacctctccattgtaagacccttttaacacagtgaatcttgttaccatgtagagcacaggacaaaaagaacacataagctcgatccttggaccctgagatgtcatatgtgctaacaggacaggggtgcccaaatcctgagcatctcagacagctggatgcgtgaccacagcagccacagtgatgtttgtggagtgtttgccacacgccacactctgctctaagggagtcatctcatttccttcttgaaacaattcccaggaggttaggaactctcaggactcaccccccacccaccccgcactgcctcttgtttttaagatggagtctccctctgttgcccaggctggagtgcagtggcttgatctcggctcactgcaacctccacctcccgggttcaagcgattctcttgcctcgaccacctgagtagctgggactacagacatgcaccagcatgcatagctaatttttaaaatttttagtagagacagggttttgctgtgttggccaggtctcgaactcctgacctcaggtgatctgcccacctcagcctcccaaagtgctgaaattacaagcgtgagccaccatgcccggcctcaggaccccatttgacagacgtgtaaactgagacctgcagcccatgcccgtgcgcctctcagcagccgtctggcttcagcgctgtgccctgaacccctgcaccaaaccacagccaggccagagagccagggcagggtttctgttctcacacagatgcaggaaggacgcacaagcccccctttgattaatttaggacctgtgcagatgcccgggacaggtagcgggagaagaggaagcaggagggaaggggtactgtggacagctggcttttctttactcacctctctggttttcttccagatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcagcttgtaatagttggtgctcgtgggcctcccctgtcgccctgaatgtccagtgtgagtccctgggctaggcagggggatctgggaggtggcccggctgggatgaggggatgaaggcaacgaggccggagcctgggccagtgtcttcaacagaattgagggacaggagcctggcgtcagggccaaggggaggggaggcctggggacagcaaaagggacagggagcggagaggtcagcttgggacccttgccctccagatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtgtgagtgagggccggaggctgggagtggagcagagaagggaccagtggcctgcctggtagtgacttcgcaccccctccccctgcccgccatgcagatgcacccaggaggctgcgtgtgtccatgagcccgggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactgtaaggcctcttcctgctcttgttcttcttggtggtggtcagtccttccttccttccttccttccttccttccttccttccttccttcctttctttctctctttcttttcctcctcctcttcttcccctcctcctcctcttcctccttcttcttcttcttctgctttctccctctcctcctcctctttcttctccttctccttccttcttcttcttcttatagagacaaggtcttactctgtcacccaggctggagtgcagtcgcataatgacacctcactgcagcctccaattcctgggctcaagcaatcctttcgcctcagcctcctgagtagctgggactatagacatgcaccaccacatccggctaatttttacatttttgtagagatggggtcttactatgttgaccaggctggtttcaaactcctgacctcaagtgacccttctgcctcagcttcccaaagccctgggattagagccatgagccactgtgcctctccctgtttcctctcatgctcctctctggccctgtctcctccagtgacttcccgaaaagccttccagaccagctggccccttcttccccacctcactgtcccctttcccattcacaccccttgctccaggccatctgccgctgtgctggaggagtcgtcttgtcctgcgtcaccagtctctctttttctacctcccctgcagtctctcctgcctccctctagggtcccctctcaggctgctccaccttccagaagtcctgagctcctactgggtccctgtctctctctctctgactactctgtctctctcagccctgacaggctccttgtttttatcttttaaaacgtaaatgattttttttttaaacggagtctcgatctgtcacccaggctggagtgcagtggcgtgatcacggctcactgcaagctctgcctcccgggttcacgccattctcctgcctcagctccccgagtagctgggactacaggcgcctgccaccatgcccggctaattttttgtatttttagtagagatgtggtttcaccgtggtctcagtctcctgacctcgtgatccgctcgcctcggccttccaaagtgctgggattacaggcgtgagccaccgcacccggctgattttttttttttttttttgagacagggtctcaccctgttgcacaggttggagtgcagtggcacaatctcagctcctgcagcctcgaccccctaggctcaagtgatactcctgcctcggcctccacagtagtaaacttgagtggtttttaaacagatcatacaagcacctggtaaaaaaataaaatgtaagcatgaaagggtaaatagtggaaagacagacgcggctcactcttgatccctgagaactctcatccccaccaaaggtcctcactgcacacagtcttcccatctcactgcacacagtcttcccatctcctccaggggtatttttcacacgcacaattcacacacagaggtctgtgcctatatacccttcctcatgacacagacgaggcagagcccacactttgcctgggaattttctttgcttttttccatgtaataatactgtttggacattgttctgtatctatgcatacacagagtgtctaaagtctggaggctgggcatggtggttcatgcctgtaatcccaacactttgagaggccatggtgggaggatcacttgagcccaggagtttgagaccagcctgggcaacatagcccacacctcatagcaggatctcatctctacaaaaaataccaaaaaaaattttaaaaacgaacaaacaaaacaataacaacaacaaaaaagagtctgaaagcatatggaagatacctaattaatttattgtactttttcagattaataattggaaacatggccttaaattgagggaacctgaccaggcacagtggctcacgcctgtaatcccagcactttgggaggccaaagtgggtggccacaagcactttgggaggccacaaggtgggtggatcacttgaggccaggagttccagaccagcctggccaatggtgaaaccccatctctactaaaaatacaaaaattagctgggtgtggtggcgcgcacctgtagtcccaactactcgggaggctgaggcaggagaatcgcttgaacccgggaggcagaggttgcagtgagccaagatctcaccactgcactccagcctgggtgaaagagcgagactctgtctcaatcaatcaatcagtcaatctagggaaccatcaccagcacggccgaaggccaaaggaaaacatctgaaacatgttatctggaaatgtaacagacacatcccatcatataaaaccattgttccctgggtatcccctttgcagacagcctgagagcctcctctttttcctggggccatggagtcctcgcgtatgaaggttgcatacatcctcctcccagtgccctcttcagggcctaggtctgcagcctcttgttttgggcacgcctgcaaggacagttgcaagatgcattcccatgtggaattgctgggtggaagacaggtgcactttcagagctgatcacacagctatactgccgtgaagagctgcacccatttatgcgggtgcgggtcacaggctctgggcgtgccacgtctgtgggaaaagcagctcctcaggagaattagtgccttattcctaaagggaggcccagagcaagcaggggccgttgtcaccctcttcctctgcacccctgcacgcatgcaggggagaaggacgagtctggctgcagagagaagtccaggatgccttccaggcaaaggctgccagggagagctggcccctcagttgattaaggtctctcctttctccacccagatagcccggagaccatcggcaggcgagtggctgtgggactcgggtcctgcctcgccatcctcatcctggcaatctgtgggctcaagctccagcgacggtgagctcctgccatcccccaccacctcctctatcccttggcagaggcccgtgtccagttgctccatctcgaagcctccagcctgtggagtccctgaccctcacacatgtgcctttatttctcagttggaagaggacacagagccagcaggggcttcaggagaattccagcggccagagcttctttgtgaggaataaaaaggtaggatggggctgggcacgatggctcatgcctgtaatcccagcactttgggaggctgaggcaggtggatcacctgaggtcaggagttcgagaccagcgtagccaacatggtgaaaccctgtctctacaaaaaatattttaaaaattagccgggcgtgatggtccatgcctgtaattccagctactcgggaggctgaggcaggagaatcgcttgaacctgggaggcagaggttgcagtgagccaagattgcgccattgcactccagcctgggtgacatactgagactctgcctcaaaaaaaaaaaaaaaaaaaaaaaaagggtaggcatgaggcagactgtgaagctgagtggggaaacaaggtgaaggaaggggataaaatgtcctctgaggagacctgggctgtcagaggccagggaaggggacgagggttggggaacaggtggttagcacttcatcctcgtctccctcccaggttagaagggcccccctctctgaaggcccccactccctgggatgctacaatccaatgatggaagatggcattagctacaccaccctgcgctttcccgagatgaacataccacgaactgggtactgagggtaccaggagggtgaccctgcaccctgggagggaggcgggaggaaaagctctgtcccgcctgccctctttgtgccaggcccatgccaggcaccctgatacatgctctgcctcattcccactcggcaacaagcctctgggtaagggccagtctccagaagtggacctgctccttcaagtcagcagcacacatgcccagtccgttcttatcacaaagatcaagacctcatgatacacacccacgcctactccctccacgcacatttgcacgagctcctactgtgagctttgggcactggggaggccacaggttttacataaggggctgaggttggggtgctgttgggggctctgggtgttgagagggaggagagttcgtgggagatgcttcatgcgtggtcgtctatctgccctgtctctcagagatgcagagtcctcagagatgcagagacctcccccggactgcgatgacacggtcacttattcagcattgcacaagcgccaagtggtaaggagggtctccccaggtctccccagaggggctgtggaaggctggggacagggcctggcctcagtggtgggtcccacataggaaggagttgggtaggcatccgtggtggcagaggttgggtgttggagacggttgctccggcagagctggccagagccaggcaggtacctccaggtcctggatgccggccacagccagtttcctgacacgaggacacccgcctgggcttttggacccccgggtggaatgaaggagagaatgcggagaaaagcggcggtggaggctggcgacagtggggccaggctaaccaccatgcggttttctcagggcgactatgagaacgtcattccagattttccagaagatgaggggattcattactcagagctgatccagtttggggtcggggagcggcctcaggcacaagaaaatgtggactatgtgatcctcaaacattgacactggatgggctgcagcagaggcactgggggcagcgggggccagggaagtccccgagtttccccagacaccgccacatggcttcctcctgcgcgcatgtgcgcacacacacacacacacgcacacacacacacacacactcactgcggagaaccttgtgcctggctcagagccagtctttttggtgagggtaaccccaaacctccaaaactcctgcccctgttctcttccactctccttgctacccagaaatccatctaaatacctgccctgacatgcacacctccccctgcccccaccacggccactggccatctccacccccagctgcttgtgtccctcctgggatctgctcgtcatcatttttccttcccttctccatctctctggccctctacccctgatctgacatccccactcacgaatattatgcccagtttctgcctctgagggaaagcccagaaaaggacagaaacgaagtagaaaggggcccagtcctggcctggcttctcctttggaagtgaggcattgcacagggagacgtacgtatcagcggccccttgactctggggactccgggtttgagatggacacactggtgtggattaacctgccagggagacagagctcacaataaaaatggctcagatgccacttcaaagaaccagtgaactcttttcagtctgtggagtcagagtggggatgaggagaggggaaagtcttcatggggcctggggggaaggaaatggagtcacagtggtttactgatcaaaccagcagattagagagtctggtgtcttgttaggaaaagcatactggctttgcagacagacaaacatgagaacttgaatcttgattcttcggcttaactagcccggtgatcttggcagggctcgccagcttgcctgagtctcggcttctccatctgtaaagtggagagggaaaggccactgcctaccacgcaggtggcccacgataggaagtgaggcatcgtcttctatatgctggagccacattgagacccagcacagaagccgaaagtcccccagaaaaccaacctccatcctttatttgcatttttcgaagaatgtgaaaaggggaagccgattcaaaacagctcttcccaaaagtgacgctacccaattcttgcacaacccatagctgccagagaaagtcttgaagaccagccaccctgagaaacctcacagcttttctgtcattcgaggtcctctgcacccgttctctaattctgttcactgattcagactttccagttaggtgccccagctcccctctccgcttctcgggtttgatctttctccacccattcacctgcttctgcaaaggggctaaagcccattctccttagagggcaggggtgtaattatctgatcatcagggagattggctcctggcacgtggcaggtgtccccaaactgccaccttcctctcccaccttcctttccctcccatctctgctcagtacccggacggtaactctaggaattatggttcagggaggccttgatcgccccatcaatccactttgtccctggtttgattcttgtgagcaccattaacttttctggaggaaggtgaggctttaagaactgtttaaagcccaggcgtggtggatcacgcctgtaatcacagcactctgggaggccaaggtgggcagatcacctgaggtcaggagttcaagaccagcctagccaacatggtaaaatcctgtctctaccaaaaatacaaaaattagctgagcgtggtgattcacacctgtaatctcagctacttgggaggctgaggcaggagagtcgcttgaacctgggaggcggaggtagcggtgagccgagatcacgccactgcactccagcctatgcaacagagtgagactccgcctcaaaacaacaacaacaaaaactgtttaaatgagacaggagggtgttggggtgtctgtgcccaaaatggggtctggtgtcattcaactctttcttcattcattctgcacacagatactgagcacctggaaatgcacccagtacagagccagttgttaggacaccaaaaagataaggcgtaggaccaaccttggggatctctagtcgagagccgccaagggtttttaaggcaagataccctggagggagctgaggcagtcctaagcctgaagggcctggcttaagctccctggcaaccatcgagcatcttccccttcccaccctgtccctcagcacagctgctgacataaacctactacatggctgattgaccatgagatatggccatcaatagacattgctgaggcgtgaagagcaggtgaagagcaggtggccaggaagctagggggccaggactccagacgcaggccgccctccacaaccccagctctcacagtcctcccagggtccagacaatggatcgtgcatttaggttcccaaacacctgctgtgcaacgtgtagtagacaaagcagaccaaagcctttgccctcaggaggccggcattctagctcaggagccagccaagacgatgtcgcactgaagaaagacaacaaaagtgagattcacaggagggatgagtgtggctgaggcacccgggaaggccctcggagaaggtgaccttgggcaaagatccagacgccaggtgggtggggccaaagcagagaagatctggaaaacccaatggctccccagggcttcgctcttgctgcaggaggggaaaccacactgctgtggattcttcctccattctgtccatttctcagttcccaacaacacactgaacagtaggaggccagttctccgctcacagcgggcgctgcaccccaggtgctgctctcgagaaaaaggcgagcatggaccaatttagtggctccgaggtatgaaaagcagtgagatggtgtctttgagggagttagtgataggctgagacccccttgctgtcccccctgaagcctggtgaccagagctgccaggaggccacacagagagaggtttatgatgggggacctggaattcggaaggggcagagtggtcaggtgacccatacacgcctgagaaaactgatgtggagagctgtgttgtcattctgaggcaggaaatagagacagactgacagacagacagaaagacacgggcacattgagagacagagacagaatcacagagcagagctggcttctcaccagcaggcgtgggcacacctgagagagcatccagagaggttagaaatcttccttaggtcctcctgggaggctcaggtgaccccagtgggaaacatgaaaggctgggaggacttccagggctgagagaaggaagctacgcagccggccggtgccacattccctgtgccttggggagtgtcccccagtgtccccagccatgcaccctggggggagtcggcttggacatctgtgccaccccttgacaggcagagtgggggccacaacctgggccctccagtgctggtcagcagaggctgcccccgccccttgccttcctcctcccgctccttcctccagccctgggaaggtcggaagccaccaccatggggctagtgcggaagcagagggagaacagaggggccctcctcgcaaaaactaaaacaagcccacagcctgtgcagaaccgcacccgggtgtgatcactccttaccccacacacccactgccacttggagatggcttcatggctgcagtcacagggcaccctgtgttgtcaggggtgagccagtgaggcccgggacctgcccaggggtgcatcaagggtggagaagacctggccccagcgcaggtgtgaacaggcaggagggctggggagggaatagcgctgggttgtggccgagcccccaagccctgctcttgagaggcactggctacacactcggcccctacccagacaggaccccaattcctccctggcccctctgtgttcctgcatcttcccagcccctcacctcctctggcgtggcctcacctccagccgggatgccccattagctgctcacccaagctgctagaacctcaggctaatggccctgtcacccgccctcctccccttccggctcactgcccatcccgggcatcctccatcagcccgctgaggggactgctgagcctcagagacatctggcagcaccaggaggtgggtccctctctctgtggctcctctcttcacctgactgttccccagacccccagaccccctgcccaggtggcccctgagaactgctaagctcaaccgtgacatggatggggcctggctgacccttgggctctccctgggggacccccttttcctgtccctggccacaccctcaccttatccacacccttctcctccctgctgccccgccggctttccaatgtgggtggggggtgggggcagctcctgccttgccagggaaccagggagggcagtgggctcaggagtcaaactcccattcccagggaaggctcaggctgtgggcccagccctgttctgaattctctccacccctcctccccacagagtctcagaaatcgagaagctttcttcctctgggacctgtgcgccacatcctatctcctgtgacgctgcaggagcttccctggttttaagaggtgacatggtggagagaaagcacaggactgggggccacaggtcctggcttctcatccaggctccgctgctccctgactgctgcctttctctgtgggcctcgtttcctccctgatcccaccaggtgaattgtaattctccacacagggctggaacccgcgagtgatcccaggagcccctcctctccggggccccaagatctgaggacagggagccaggttgcacacaggagccgtgcaggccaggacggcggccccatggacctgcccccgcagctctccttcggcctctatgtggccgcctttgcgctgggcttcccgctcaacgtcctggccatccgaggcgcgacggcccacgcccggctccgtctcacccctagcctggtctacgccctgaacctgggctgctccgacctgctgctgacagtctctctgcccctgaaggcggtggaggcgctagcctccggggcctggcctctgccggcctcgctgtgccccgtcttcgcggtggcccacttcttcccactctatgccggcgggggcttcctggccgccctgagtgcaggccgctacctgggagcagccttccccttgggctaccaagccttccggaggccgtgctattcctggggggtgtgcgcggccatctgggccctcgtcctgtgtcacctgggtctggtctttgggttggaggctccaggaggctggctggaccacagcaacacctccctgggcatcaacacaccggtcaacggctctccggtctgcctggaggcctgggacccggcctctgccggcccggcccgcttcagcctctctctcctgctcttttttctgcccttggccatcacagccttctgctacgtgggctgcctccgggcactggcccgctccggcctgacgcacaggcggaagctgcgggccgcctgggtggccggcggggccctcctcacgctgctgctctgcgtaggaccctacaacgcctccaacgtggccagcttcctgtaccccaatctaggaggctcctggcggaagctggggctcatcacgggtgcctggagtgtggtgcttaatccgctggtgaccggttacttgggaaggggtcctggcctgaagacagtgtgtgcggcaagaacgcaagggggcaagtcccagaagtaacgccactgctcgggggaaggagcatggggcaggagggcccggctgcttctccaggcccctgcggggggctgcttcggaggaactgcagggcagcctggcccggaggcctccctggagccactcaagcagagagcggcgcctgctgagggcagcaccccagtcaagagaggagcaccgagccagagcacggtggcagggggaggtaagtttgcccctgcacgtgtcgaggaagtttgtcccttcctcgcctatctttctctcccctctgcacgtcctcacctgcctgtcttccgtgggccgcgagcagaggccttgtacttacgggaaggaggaggcagtctgtttctgacccacaggaactgccactccggtgatgcacttgaggacagctactctgaaatcagttcccaggagagaacatttgatttccgagtaggagaggaggcaggaggcaggataatgacgctgttataccatgtgaaactcgcaagggggcagccttggagcttggaacggaggctggcaggaaaagatgcttcagccggggagggaaaatgcaaggtcccgcagtgatgggcaaaggctacggagttgactcttgaacaacgtggggatggggtgccgaaccccacgcagtggaaaatctgtgcatgactttgactcccccaaagttcaactactaatagcctactgttgaccaaaagctttacccagtatataaacaattgagtaagacatatttgaagccaggcacagtggcttatgcctgtaatcccagcactttaggaggccgaggcgggaagactgctcgaagccaggagttcaagagcagcctgggcaagatagtgagacccccacctctacaaataataaaaataaaaataattagctgggcgtggtggtgcgtgcctgtagtcccagctactcaggggtctgaggtgggaggatcccttgagcccaggattgaggctgcaatgccatgatcgcaccactgcactccagcctgggtgacagagcaagatcctgtctctggaaaaaaaagaaaaaagacatattttgtatgttatatatattatgtgctgtattcttacaataaagtaagctagagaaaagtacatgttactgaaaacattattagaaagagaaaataccttcacagcactgtgctgtatttatcaatactgtaagttttgttgtctgtttataagagaagttgtctgtctcaagcggcagtaaccgcggctgcagatctcaatctgtggtagatatcaagcagttcaacccttttttataatgtcatgacttttctttgcttcttggaacagctctggcatcactagtggccccgcatttggggccaagggggttattcaaagtttacggtattgcattaaacatggtgaaacatatgcaatgactatgaaagatcactttttacttctgtacacagtgtactggcgagaactgctcaccaggaggtgattagcttcacgtggtgttggaagcagatactcaacagttgagctcacggagaaagcacaggaggtggctacgaaattattataggagtacagtatgtctacagtaaatatgcacttatgactttaatactgcacatctttatgtttgcttgcatttctcttgacttcaaatggcatcaaatagagtttgtgtttgcgtgcatatatttttgataaattttaacttttttgttttgttttgtttttagacagagtcttgctctgtctcccaggctggagtgcaatggtgtgatcttggctcactgcaacctccgcctcccaggttcaagcaattctcctgcctcagcctcccaagtggctgggattacaggcacctaccaccacacctggctaattttttgtattgtaagtagagatggggtttcaccatgttggccaggcctgtctcgaacccctgacctcagttgacccacccgcttcggcctcccaaagtgctgggattacaggcgtgaaccacggtaccctgccaattttaactttctataagagatttgcccagcctgggcaacatggagaaaccctgtctttaccaaaaatacaaaaaatttagctgggcatggtggcgtgtgcctgtagtcccaggtacttgggaggctgaggtggaaggatggtttcagcctgggacgcagaggttgcagtgagccgagatcatgtcattgcactccagcctgggtaatagagccagatcctgtctcaaaaaaaaaaaaaaaaaaagctttgtatccattttggcatacatagactagtatgtatggcagtaaatagactagtggcagtaaatagactagtacctacatatattttttgtattcgtggcatacttaacttttgcttaattttaaaaatatatttctaggcttgtagttcctctgtaagttttttcaaattgttgcaaatctctgaaaatgtttccaatgtatttattgaaaaaaatccacaagtaagtggacctgcacagttcaaacctgtgctattcaaaggtcaactatatagtctgtaatacgtgatgggtgtcacgctgagtgtctacagtgagtgttgtctcacttcagcttcacacaacgctgtggtttagccttgccactgccgtacggatgagcgggtgttcactacacgaaggtactgggcagaggagcaaacactgactaaattctagcctgtgctttgctcaacaagccacatgcctgccttgcttgcatcaagggaaggagtattgtgttctaatttgaagaaatacaccagccactccttaggcagcacttggtgcgggatgggctcctctggaaagggagcacctaatttgcacaaaggtgctgcaccaggtagctacgatcctggaatgggatgaagaaacttgcctaaggccctacagcaaggaaggggtaaaagcaggatgtgatccttccaagcagaacccccaagagccaggggacatttgggaaacctgggggtgtgggggttaaccctgtatttgtggtccatttcgcaaaggatagcatcccacatccatgagagggaggtcagaagggagtggctgtggctgtgaaggagacagacacctgggcacctgctctaaattacagcaatgccatgcacatgatgatgtattggagcacaacaaatggcagccaccagctggggtgcacgcaccaatttctacaaatgagctctgttcaggctgaaatgtctacgcatgcaaggatttttcttgtaattgatgatatatccccagcgcccagaagacagagtggcacgcagtaggtgctcagtaaatgttcgctcaatgaatgaatgaatgcctctactagccttccaaagaaagttctatgtctgttttatcaatatggaaacaggccagagaaattaagtaacttcttttggaagccagaaacaagcccggcacttgtgcagtgacaacaccagggaatttcagtaaaaacccaaactccaccccagggagccccagtgcctcctcccgcatcacagggtgccagggcccactctctttgactcctgctccatagcccctgctgggtactgggctgcttctcccagttcctcctgcctccccccacttagggcaaatccagtgcctccctggggcccaggagactcagtgtgatccaccccaggcgcctctcactcctgtgagacccaaacctcctatcagctcccccagcaggccaagtctgtgcacccgtgactcccctgccccaatgttccctccagtggctctttgcctcttcagtatccacgtcttcatgatccttcccctactccatcccccaagccctcaccaccaaatgtaatgtaaacagcatgacagcaaaaactttttgttcacaactgcgtgttgttttttttttttttttttttttttagacacagtctcactctgttgctcaggatggagtgcagtgacacagtcatggctcactgcagccttgaattcctgggctcaagcgattcttccatttcagcttcccaagtagctgggaccagaggcacatgccactaaacccagctaatttcttttttctttctctcttttttttttttttttcccctgtggacatagggtctcctcatgttgcccaggctagtctcaaacaacctgtgctcaagcaatcctcccacctcagcctgccaaagtgctaaaattacggacctgagccaccgtacccagcctactgctgcacttttaacagcttaattgagatattatgtacatatcatcaaattcaccaatgttaagtgtacaattcaatgatttttagtaaatttactgaggtgtgagtccattaccatcagctagttttagaacattttcatgaccctagtaaaggattttacttcatgttcatttaaacttaatcttgggccaggcggggtggctcacgcctataatctcagcattttgggaggctggggtggacggatcacctgaggtcagtagttcgagaccagcctggccaacatggcgaaacctcatctctactaaacatacaaaaattagccaggcgtggtggcgggcacctgtaatcctagctacttaggaagctgaggcatgagaatttcttgaacccgggaggcagagtttgcagtgagccaagattgcaccattgcactccagcctgggcgacaacaagaccctgtctcaaaaaaaaaaaaaaaaaaaaaaaaacaacttaatcttcatttctacctccatcccccagacaaccacgagtctactttctgactctataactttgcctattctggacatatcatataaataaatgaaatctgacaatatatgtacatggtcttttacgtctggcttgtttcactcaacataaggctgtagtgtgtatgggtagctcattccttttctttgccaaatagaattccagtatatggatgtgccacttttttattcactcaccagctgatgggctgccaggtaacatttccactgttggtgactatgaccaacactgctgtgaacatttaggactgagtctttaagtagacctgagtttccacttctcttaagtaaatacctacggtggaattgttggatcgcatggtgaatttatgtttaactttttaagcaactgccacctgctttccaaagcagggacaccacctcccattattctcaccagcagtgaatgaggcccctatctctctacattctcgccgaccataggcattgtctgactttttgcttgtggccattctagtggatgtgaaatcatatctcattgtgccctgtgattgtttgaatgaactaatagatcacctgtgaaactggacagcacttgggtttgaaccctagtggtgacatttcacaagctgtatcacctggagcaagtcacttccctacggtgaaggacaaggattttatgacataaaagcgttgccctagaagcagtgtctgctatggattatgtgcccatgaaaaggcagctgctgtcatcatcgccaccaccattattattgttattattattacattgtcaccccagagataatcctgcaccagtgacagggaaaatagcagctggcatccgctgcctgctcacgaccacacgccaggcttccgtcaaaccactcaacatgtattagtaatcttttaatcgacctacatattgttttaatttgcatgtgttaatgcgttgatctatgagatgggtattatgatgagctctgttctgcaggggagaaagcagaaacatggagaatttaagtcatttcccccaaatcacaaagtcaggaagaaacagacctcacggagctcgctctctgtcattgcatcacacttcctgcccttacaaggcaaattggataaatgccattctagagaagcagacaaaattcaagtgaagaaggggagaggaagacgtcggctggggcctgcttagagcatcccagctgagactgcatgaggagggaggcacgcagttgtggaatttgttccccttttagcatgctgaccagccctggcaacggagctcaaggcatctatgtgccactgctcaacagtgagtgacgtcatgggcacggccaggtctttatcagttctgccggataaatagccaactgcactaggtctggagagacagcaaggtgctgtgcggcagagcatttggggtctcaaagaagcaggtgagcctgggcccgaggggctgggtggaggagcaccttggtgcttctctgctggggaagggacaggggacagggcatgctcaggaagacaggcaggctgaccccgcctggaaggcacccagagacaagaggggtgggcgtagtgacctcgtgcccttttaggggagatgctgctggccagaggccgttagggcccccactaccaactccatgttactctctctcaccagtggccaccaccatggatacaggccccgaccagtcctacttctccggcaatcactggttcgtcttctcggtgtaccttctcactttcctggtggggctccccctcaacctgctggccctggtggtcttcgtgggcaagctgcagcgccgcccggtggccgtggacgtgctcctgctcaacctgaccgcctcggacctgctcctgctgctgttcctgcctttccgcatggtggaggcagccaatggcatgcactggcccctgcccttcatcctctgcccactctctggattcatcttcttcaccaccatctatctcaccgccctcttcctggcagctgtgagcattgaacgcttcctgagtgtggcccacccactgtggtacaagacccggccgaggctggggcaggcaggtctggtgagtgtggcctgctggctgttggcctctgctcactgcagcgtggtctacgtcatagaattctcaggggacatctcccacagccagggcaccaatgggacctgctacctggagttccggaaggaccagctagccatcctcctgcccgtgcggctggagatggctgtggtcctctttgtggtcccgctgatcatcaccagctactgctacagccgcctggtgtggatcctcggcagagggggcagccaccgccggcagaggagggtggcggggctgttggcggccacgctgctcaacttccttgtctgctttgggccctacaacgtgtcccatgtcgtgggctatatctgcggtgaaagcccggcgtggaggatctacgtgacgcttctcagcaccctgaactcctgtgtcgacccctttgtctactacttctcctcctccgggttccaagccgactttcatgagctgctgaggaggttgtgtgggctctggggccagtggcagcaggagagcagcatggagctgaaggagcagaagggaggggaggagcagagagcggaccgaccagctgaaagaaagaccagtgaacactcacagggctgtggaactggtggccaggtggcctgtgctgaaagctaggtcctccgggggaggagggtgtagctggcatgtcatcctcagggcgcttcctcgctcacgccaggagggacttggagtggcgagctggggcccgatggggcttgggggcagagtagacatctagcctccctaagggtatgcgcgctaaagcccagctctcgatctcacctccatccccatccacccacacactatggattgggctctgggaaggggtcagggtgagaggctgctctggagaacaatgaggtcctcatagcagcaggcagctcctgtgttttcttgagggtggcagaggagctaagagcagtgcccagggtctgagggggctgcccagtgagtggcaggggcaggagaggggagaaacttctccttttgctctcagatgctgccagggtccctgaagagggaagacacgcggaaacaggcttgcacccagacacgacaccatgcatctcctcggcccctggctcctgctcctggttctagaatacttggctttctctgactcaagtaaatgggtttttgagcaccctgaaaccctctacgcctgggagggggcctgcgtctggatcccctgcacctacagagccctagatggtgacctggaaagcttcatcctgttccacaatcctgagtataacaagaacacctcgaagtttgatgggacaagactctatgaaagcacaaaggatgggaaggttccttctgagcagaaaagggtgcaattcctgggagacaagaataagaactgcacactgagtatccacccggtgcacctcaatgacagtggtcagctggggctgaggatggagtccaagactgagaaatggatggaacgaatacacctcaatgtctctgaaaggccttttccacctcatatccagctccctccagaaattcaagagtcccaggaagtcactctgacctgcttgctgaatttctcctgctatgggtatccgatccaattgcagtggctcctagagggggttccaatgaggcaggctgctgtcacctcgacctccttgaccatcaagtctgtcttcacccggagcgagctcaagttctccccacagtggagtcaccatgggaagattgtgacctgccagcttcaggatgcagatgggaagttcctctccaatgacacggtgcagctgaacgtgaagcacaccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaagtactgctgtcaggtctccaatgacgtgggcccgggaaggtcggaagaagtgttcctgcaagtgcagtatgccccggaaccttccacggttcagatcctccactcaccggctgtggagggaagtcaagtcgagtttctttgcatgtcactggccaatcctcttccaacaaattacacgtggtaccacaatgggaaagaaatgcagggaaggacagaggagaaagtccacatcccaaagatcctcccctggcacgctgggacttattcctgtgtggcagaaaacattcttggtactggacagaggggcccgggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaaccccatggcgcctgggaggagccatcgcttggggtgctgaagatccaaaacgttggctgggacaacacaaccatcgcctgcgcagcttgtaatagttggtgctcgtgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagcccgggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactatagcccggagaccatcggcaggcgagtggctgtgggactcgggtcctgcctcgccatcctcatcctggcaatctgtgggctcaagctccagcgacgttggaagaggacacagagccagcaggggcttcaggagaattccagcggccagagcttctttgtgaggaataaaaaggttagaagggcccccctctctgaaggcccccactccctgggatgctacaatccaatgatggaagatggcattagctacaccaccctgcgctttcccgagatgaacataccacgaactggagatgcagagtcctcagagatgcagagacctcccccggactgcgatgacacggtcacttattcagcattgcacaagcgccaagtgggcgactatgagaacgtcattccagattttccagaagatgaggggattcattactcagagctgatccagtttggggtcggggagcggcctcaggcacaagaaaatgtggactatgtgatcctcaaacattgacactggatgggctgcagcagaggcactgggggcagcgggggccagggaagtccccgagtttccccagacaccgccacatggcttcctcctgcgcgcatgtgcgcacacacacacacacacgcacacacacacacacacactcactgcggagaaccttgtgcctggctcagagccagtctttttggtgagggtaaccccaaacctccaaaactcctgcccctgttctcttccactctccttgctacccagaaatccatctaaatacctgccctgacatgcacacctccccctgcccccaccacggccactggccatctccacccccagctgcttgtgtccctcctgggatctgctcgtcatcatttttccttcccttctccatctctctggccctctacccctgatctgacatccccactcacgaatattatgcccagtttctgcctctgagggaaagcccagaaaaggacagaaacgaagtagaaaggggcccagtcctggcctggcttctcctttggaagtgaggcattgcacggggagacgtacgtatcagcggccccttgactctggggactccgggtttgagatggacacactggtgtggattaacctgccagggagacagagctcacaataaaaatggctcagatgccacttcaaagaaaaaaaaaa

SEQ ID NO: 9 - DNA, 20 bp, Artificial Sequence
  Primer
cacagctatactgccgtgaa

SEQ ID NO: 10 - DNA, 20 bp, Artificial Sequence
  Primer
cacagctatactgccgtgaa

SEQ ID NO: 11 - DNA, 22 bp, Artificial Sequence
  Primer
gctccttcaaggagaattagtg

SEQ ID NO: 12 - DNA, 20 bp, Artificial Sequence
  Primer
ggcatgaggcagactgtgaa

SEQ ID NO: 13 - DNA, 20 bp, Artificial Sequence
  Primer
aacctctgccaccacggatg

SEQ ID NO: 14 - DNA, 20 bp, Artificial Sequence
  Primer
ccactcggcaacaagcctct

SEQ ID NO: 15 - DNA, 20 bp, Artificial Sequence
  Primer
gaaggagcaggtccacttct

SEQ ID NO: 16 - DNA, 20 bp, Artificial Sequence
  Primer
cacagccagtttcctgacac

SEQ ID NO: 17 - DNA, 50 bp, Artificial Sequence
  Primer
agggaccctggcagcatctgagagcaaaagttctttgaagtggcatctga

SEQ ID NO: 18 - DNA, 20 bp, Artificial Sequence
  Primer
ccagccccaaaccgaaagtc

SEQ ID NO: 19 - DNA, 19 bp, Artificial Sequence
  Primer
ccaggggccgaggagatgc

SEQ ID NO: 20 - DNA, 21 bp, Artificial Sequence
  Primer
gatgtgctccaggctaaagtt

SEQ ID NO: 21 - DNA, 21 bp, Artificial Sequence
  Primer
agaaacggaatgttgtggagt

SEQ ID NO: 22 - DNA, 24 bp, Artificial Sequence
  Primer
ccagccccaccaaaccgaaaagtc

SEQ ID NO: 23 - DNA, 18 bp, Artificial Sequence
  Primer
ccaggggccgaggaatgc

SEQ ID NO: 24 - DNA, 21 bp, Artificial Sequence
  Probe
cctgcctcgccatcctcatcc

(19) United States
(12) Patent Application Publication
(10) Pub. No.: US 2012/0207758 A1
(43) Pub. Date: Aug. 16, 2012

(54) CD22 Exon 12 Deletion Mutants

(76) Inventor: Fatih M. Uckun, White Bear Lake, MN (US)

(21) Appl. No.: 13/230,843

(22) Filed: Sep. 12, 2011

Related U.S. Application Data
(60) Provisional application No. 61/381,938, filed on Sep. 11, 2010.

(51) Int. Cl. A61K 39/395; C07H 21/04; C07K 16/28; A61P 35/02; A61K 31/7088; C12Q 1/68; C07K 7/08; B82Y 5/00

(52) U.S. Cl. 424/139.1; 435/6.11; 536/24.31; 536/24.5; 530/326; 530/387.9; 514/44 A; 977/906; 977/904

(57) ABSTRACT

Provided herein are CD22ΔE12 polynucleotides and polypeptides, as well as diagnostic compositions and methods for identifying patients suffering from B-cell disorders such as leukemias, and particularly for identifying aggressive disease. Use of the disclosed association of specific CD22ΔE12 gene and polypeptide mutants with specific disease and prognosis also provides new targeted therapies for the associated disorders.

### CROSS-REFERENCE TO RELATED APPLICATION

This application claims priority to U.S. Provisional Application No. 61/381,938, filed Sep. 11, 2010, which is herein incorporated by reference in its entirety.

### BACKGROUND

B-precursor leukemia (BPL), the largest subset of acute lymphoblastic leukemia (ALL), is the most common form of childhood cancer. Despite recent improvements in treatment outcome of childhood BPL, infants with BPL continue to have a disappointingly poor treatment outcome even after intensive chemotherapy and supralethal radiochemotherapy in the context of hematopoietic stem cell transplantation.

Although mixed lineage leukemia (MLL) gene rearrangements were originally thought to play the key role in the leukemogenesis and poor prognosis of infant BPL, the failure of these defects to cause leukemia in transgenic or knock-in mice, the absence of universal concordance of BPL in infant monozygotic twins with MLL rearrangements, and clinical biomarker studies in newly diagnosed infant BPL patients revealed that MLL rearrangements are not sufficient to explain the leukemogenesis or aggressive biology of infant BPL. These observations support the notion that yet undefined molecular abnormalities contribute to the uniquely aggressive biology and poor outcome of infant BPL.

There is a continuing need to identify biomarkers for aggressive leukemias, particularly infant BPL, as well as identifying genetic causes that permit early detection and intervention, including targeted therapies.

### SUMMARY

The inventors have now isolated and purified CD22 mutant polynucleotides encoding CD22 mutant polypeptides having of all or a portion of CD22 Exon 12 (E12) deleted. The CD22 Exon-12 deletion mutants (CD22ΔE12) have been identified and shown in the Examples below to be a genetic defect implicating a B-cell co-receptor in the pathogenesis and biology of a human disease, and particularly in leukemia. Significant up-regulation and significantly higher expression of CD22ΔE12 mutant, of associated mutations in CD22 Intron 12 (positioned between Exons 12 and 13, CD22ΔI12), and of a newly identified CD22ΔE12-associated signature transcriptome, described, for example, in the working Examples below, demonstrate the utility of the mutant CD22 molecules as biomarkers of leukemia, and particularly of aggressive B-cell disease, including BPL.

As discussed in the Examples below, the CD22 mutations have been found to include those having one or more, for example, a plurality of mutations in CD22 intron 12 positioned between exons 12 and 13 (CD22ΔI12) that induce changes in the splice machinery for CD22, resulting in splice mutation and deletion of all or a portion of CD22 exon 12. The Examples below demonstrate the newly-discovered association of the mutant biomarker(s) with aggressive leukemic disease, and particularly with B-lineage leukemic disease.

Specific evidence of utility includes demonstration in two large ALL patient cohorts that primary leukemia cells (PLC) obtained from relapsed pediatric B-lineage ALL patients expressed significantly higher levels of the new CD22ΔE12—associated signature transcriptome than PLC obtained from newly diagnosed pediatric B-lineage ALL patients. In addition, comparison of matched pair initial diagnosis versus first relapse leukemic specimens demonstrated significant up-regulation of the CD22ΔE12 deletion mutant and the associated signature transcriptome in clones obtained from relapsed patients. Further, PLC from 19 of 19 pediatric ALL patients with a first bone marrow relapse within the first 12 months of completing primary therapy exhibited the CD22ΔE12 biomarker. Similarly, PLC obtained from diagnostic initial bone marrow specimens from 7 of 7 therapy-refractive newly diagnosed pediatric B-lineage ALL patients with less than 7 months event free survival (EFS), including 4 with induction failures and 3 with early relapses, were positive for the biomarker CD22ΔE12. In contrast, only 1 of 5 PLC samples from newly diagnosed pediatric B-lineage ALL patients with greater than 18 months EFS was positive for this biomarker.

RT-PCR analysis described herein of PLC specimens in matched-pair diagnosis versus induction failure (day 28 bone marrow with M3 status) as well as PLC in diagnosis versus first bone marrow relapse specimens, provided direct evidence that the CD22ΔE12 genetic defect is detectable in PLC of therapy-refractive pediatric ALL patients both at the time of initial diagnosis and at the time of documented treatment failure. These data described herein, including the working Examples, implicate the CD22ΔE12 and CD22ΔI12 genetic defects in newly diagnosed infant ALL, and in the aggressive biology of relapsed and/or therapy-refractory leukemia, particularly in pediatric ALL patients. Accordingly, the CD22 E12 deletion mutations, I12 intron mutations, and associated signature transcriptome provide useful biomarkers for the diagnosis of leukemia, identification of risk for aggressive disease, for example, in the evaluation of remission bone marrow specimens for the presence of residual therapy-refractory clones and/or the identification of patients at high risk for treatment failure.

Particular embodiments of the invention include methods, systems, probes, kits, and the like, that utilize one or more of the CD22 mutation biomarkers and/or gene signature transcriptome for identifying risk, presence, diagnosis and/or prognosis of leukemia, for producing therapeutic molecules targeting the CD22ΔE12 defect and thereby treat disease associated with the defect, for example, B-cell disorders, including leukemia, and particularly BPL. In one embodiment, the presence and/or amount of a CD22ΔE12 deletion and/or CD22ΔI12 mutation(s) inducing exon 12 deletion and/or the gene signature transcriptome described herein, indicates a patient has or is at risk for developing aggressive leukemic disease. In alternative embodiments, the presence and/or amount of a CD22ΔE12 deletion and/or CD22ΔI12 mutation(s) inducing exon 12 deletion and/or the gene signature transcriptome described herein, indicates a patient's response to chemotherapy, bone marrow transplant, radiation therapy, hematopoietic or cord blood stem cell transplant, or other therapy for treatment of leukemia.

Diagnostic molecules and methods for detecting one or more of the CD22ΔE12 deletion mutations and/or one or more of the CD22ΔI12 mutations and/or the CD22ΔE12-associated gene signature transcriptome for the diagnosis of disease risk, presence, severity, prognosis, response to therapy, production, and screening of therapeutic molecules, and the like, are provided herein. CD22ΔE12 positive leukemia cells can be detected by one of many known methods for detecting genetic biomarkers of disease, that include, for example, use of RT-PCR, confocal immunofluorescence microscopy, FRET analysis, labeled oligonucleotide probes, dual labeled molecular beacons, for example, reactive with Exons 12 and 13 individually or with the Exon 12-Exon 13 junction specific to the CD22ΔE12 mutation, antibody based peptide detection methods, for example, directed to the unique 15-amino acid peptide of the CD22ΔE12 mutant described herein, splice-sensitive microarrays, for example high density microarrays, deep-transcriptome sequencing, and high throughput DNA sequencing. The diagnostic methods can be performed on one of many known biologic samples obtained from a subject, including, for example, cells, tissue, and/or fluids obtained from the subjects bone marrow, blood, bone, cerebrospinal fluid, cord blood, and the like. CD22ΔE12 positive leukemic cells can also be detected by methods used to identify the CD22ΔE12 deletion mutant nucleic acid or amino acid molecule or portion thereof, the CD22ΔI12 intronic mutant nucleic acid or amino acid molecule or portion thereof, and/or the CD22ΔE12 associated gene expression profile, as described herein.

Methods for interfering with the expression of the target CD22ΔE12 genetic defect, abnormal RNA species, and/or truncated CD22 protein, such as by administering targeted antibodies, sense and antisense oligonucleotides, spliceosome-mediated RNA transsplicing, exon-specific splicing enhancements, nanoparticles and/or other formulations loaded with antisense or siRNA sequences targeting CD22ΔE12, and other such therapeutic molecules are also provided.

The Examples further disclose the identification of specific genetic signatures useful for the diagnosis of B-cell disease risk, presence, severity, prognosis, and the like, and also provides pathways for the development and/or use of targeted therapeutic treatments based on the newly identified CD22ΔE12 mutations.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows expression of a truncated CD22 receptor in cells obtained from infant BPL patients, and identified as described in Example 1. Panels A-D show results of CD22 Western blotting of whole cell lysates of primary leukemia cells from 6 infant BPL patients and a pediatric BPL patient (PT7) (panels A-C) as well as control lysates obtained from RAMOS, DAUDI, FL.8.2⁺, and FL8.2⁻ cell lines (panels D and E). The absence of apoptotic ladder-like DNA fragmentation induced by treatment with the anti-CD22 monoclonal antibody, HB22.23 in infant BPL cells as compared to the presence of apoptotic ladders in treated controls is shown in panels F and G.

FIG. 2 is a series of blots showing the results of a genomic PCR analysis of human CD22 gene exons 10-14 designed to identify genomic changes that might induce the identified CD22 truncation mutant shown in FIG. 1. Samples of amplified nucleic acid sequence included an 885-bp PCR product spanning CD22 exons 10 and 11 and the exon-intron junctions (Panels A.1, B.1); a 905-bp PCR product spanning CD22 exons 12 and 13, and the exon-intron junctions (A.1, A.2, B.2); and a 964-bp PCR product spanning exon 14 and its exon/intron junctions are shown (A.2). PCR products from 6 infant BPL patients (PT1-PT6) and controls including two pediatric BPL patients (PT7 and PT8), the Burkitt's/B-ALL leukemia/lymphoma cell lines RAJI and DAUDI, BPL cell lines REH (Pre-Pre-B), and NALM-6 (Pre-B), normal fetal liver B-cell precursor cell line FL8.2⁻ (Pro-B), and the EBV-transformed B-lymphoblastoid cell line BCL-4 are shown. Empty lanes are shown with a dash. Normal size PCR products are indicated by the arrowheads.

FIG. 3 shows the normal genomic sequence of exon 12 and its surrounding intron/exon junctions, as obtained from NCBI Reference Sequence: NC_{—}000019.9. Coding sequence is shown in upper case and non-coding intronic sequence is depicted in lower case. The splice donor and acceptor sites are underlined. Sense and anti-sense genomic PCR primers are indicated by the direction of the arrows, right or left, respectively.

FIG. 4 is a sequence alignment comparing intronic mutations of the CD22 gene in infant B precursor leukemia cells (PT1, PT3, PT5, PT6). Panel A aligns the intronic segment between exons 11 and 12 starting at position NC_{—}000019.9: c.2208-83G=g.35,836,421G and ending at position NC_{—}000019.9: c.2208-1G=g.35,836,503G plus a short segment of exon 12 between c.2208 and c.2214 in primary leukemic cells from 4 infant BPL patients (PT1, PT3, PT5, PT6). Panel B aligns the genomic sequence of exon 12 in primary leukemic cells from 4 infant BPL patients (PT1, PT3, PT5, PT6). The genomic sequence, starting at position NC_{—}000019.9: c.2208-15T=g.35,836,490T and ending at position NC_{—}000019.9: c.2327+26C=g.35,836,649C) is shown for each patient in comparison to the wild-type (WT) sequence. Panel C aligns the intronic segment between exons 12 and 13 starting at position NC_{—}000019.9: c.2327+29G=g.35,836,652G and ending at position NC_{—}000019.9: c.2328-1G=g.35,837,053G. Locations of genomic DNA sequence mutations in the intron segment are shown boxed.

FIG. 5 shows the predicted secondary structures of the mutant CD22 pre-mRNA sequences in infant BPL cells. Panel A.1 shows the predicted folded structure for the wild-type CD22 pre-mRNA sequence with the target motifs for the splicing factors hnRNP-L, PTB, and PCBP. Positions of the misalignments caused by genomic mutations for each patient are indicated by arrow symbols. Panel A.2 shows the predicted folded structure of wild-type CD22 pre-mRNA as compared to the predicted folded structure of CD22 pre-mRNA in infant BPL cells. Panels B.1 and B.2 show binding motifs for hnRNP-L. Panels C.1 and C.2 show PTB binding motifs. Panels D.1 and D.2 show PCBP binding motifs.

FIG. 6 shows an RNA sequence alignment of the pre-mRNA sequence corresponding to the intronic sequence between Exons 12 and 13 (CD22ΔI12). Genomic DNA sequence for the wild-type consensus sequence and patient sequences were converted to the sequences of the positive strand RNA complement for alignment.

FIG. 7 shows an identified CD22 Exon 12 splicing defect in transcripts obtained from infant B-precursor leukemia cells. Panel A shows a restriction map of CD22 cDNA. Arrows designate the location of RT-PCR oligonucleotide primers, 22-1 and 22-2, used to amplify a 975 base pair product encompassing a sequence encoding the CD22 transmembrane and cytoplasmic domains. Panel B shows results of RT-PCR analysis of control, FL8.2-negative fetal liver derived non-leukemic B-cell precursors as compared with infant BPL cells obtained from patients, PT1, PT3, and PT5. Panel C shows results of Southern blot analysis of the CD22 PCR products shown in (B) using an oligonucleotide probe specific for exon 11. The positions of the CD22 RT-PCR products are indicated with arrow heads. Panel D shows results of EcoRI restriction analysis of cloned CD22 RT-PCR products from control FL8.2⁻ cells. Panels E and F EcoRI show results of restriction analysis of cloned CD22 RT-PCR products from primary infant BPL cells from PT1 (E) and PT3 (F). Panel G shows Sequence chromotographs of the wild-type and mutant CD22 RT-PCR products. Panel I provides a nucleic acid sequence of three Exons: 11, 12, and 13 and the aberrantly spliced mRNA translated sequence of CD22ΔE12.

FIG. 8 demonstrates the generation of transgenic mice harboring a human CD22ΔE12 gene. Panel A is a schematic diagram of the transgene construct. Panel B is a Southern blot showing results of genomic DNA analysis of the founder mice. M: size markers, 1-kb DNA ladder. Positive control=transgene construct. Negative control=genomic DNA from a non-transgenic mouse. Panel C shows results of Dual color FISH analysis of metaphase chromosomes from bone marrow cells of an hCD22ΔE12-Tg mouse showing the human CD22ΔE12 transgene on one chromosome 14 of the diploid set. Panel D shows reverse DAPI karyotyping of chromosomes obtained from chromosomes shown in C. Panel E shows Dual color FISH analysis of metaphase chromosomes from bone marrow cells obtained from an hCD22ΔE12-Tg male mouse showing the hCD22ΔE12 transgene on sex chromosome X. Panel F shows Reverse DAPI karyotyping of chromosomes obtained from E. Panel G shows results of genomic CD22 transgene PCR analysis of splenocytes from the hCD22ΔE12-Tg mice. Positive control=genomic DNA from a founder mouse. Negative control=genomic DNA from a non-Tg control mouse. Mouse β-casein exon 7 was used as an internal control for DNA integrity and PCR efficiency. Panel H shows an Exon 11 Southern blot of CD22 exon 12 RT-PCR products from splenocytes obtained from transgenic mice. Positive control=PCR product from intact human CD22 cDNA. Negative control=non-transgenic FVB mice. Panel I shows results of Western blot analysis of splenocytes from CD22ΔE12 transgenic mice probed with an anti-N-terminal CD22 antibody. Panel J shows the DAUDI cell line control Western blot probed with the anti-C-terminal CD22 antibody.

FIG. 9 shows B-precursor/B-lymphocyte hyperplasia in CD22ΔE12 transgenic mice. Total numbers of B-lineage lymphoid cells in spleen and bone marrow samples obtained from 9 hCD22ΔE12 transgenic mice and 3 control FVB mice were determined by flow cytometric immunephenotyping using monoclonal antibodies to murine B220 and CD19 surface pan-B cell antigens and polyclonal antibodies to murine IgM.

FIG. 10 shows differential expression of the twelve hCD22ΔE12-associated signature genes in transgenic mice and infant ALL patients.

FIG. 11 is a table showing the expression of the 12 gene signature in splenocytes transfected with hCD22ΔE12 and in ALL patients.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

### CD22 Exon 12 Deletion

CD22ΔE12 is the first reported genetic defect implicating a B-cell co-receptor in the pathogenesis and biology of a human leukemia as well as linking homozygous mutations of the CD22 gene to a human disease. Analysis of the gene expression profiles of two large ALL patient cohorts provided previously unknown evidence that PLC (primary leukemia cells) obtained from relapsed pediatric B-lineage ALL patients have significantly higher expression levels of a CD22ΔE12-associated signature transcriptome than PLC obtained from newly diagnosed pediatric B-lineage ALL patients. Furthermore, comparison of matched pair initial diagnosis versus first relapse leukemic specimens revealed a statistically significant up-regulation of the CD22ΔE12-associated signature transcriptome in relapse clones.

In agreement with and validating the results of the gene expression profiling, PLC from 19 of 19 pediatric ALL patients in first bone marrow relapse occurring within 12 months of the completion of primary therapy were found to be CD22ΔE12-positive. Likewise, PLC in diagnostic initial bone marrow specimens from 7 of 7 therapy-refractory newly diagnosed pediatric B-lineage ALL patients with less than 7 months event free survival (EFS), including 4 patients with induction failures and 3 patients with early relapses, were found to be CD22ΔE12-positive, whereas PLC from only 1 of 5 newly diagnosed pediatric B-lineage ALL patients with greater than 18 months EFS was CD22ΔE12-positive.

RT-PCR analysis of PLC in matched pair specimens of diagnosis versus induction failure (day 28 bone marrow with M3 status) and PLC in matched pair specimens of diagnosis versus first bone marrow relapse provided direct evidence that a CD22ΔE12 genetic defect is detectable in PLC of therapy-refractory pediatric ALL patients both at the time of initial diagnosis as well as the time of documented treatment failure. Based on this compelling experimental evidence, CD22ΔE12 expression appears to confer ALL cells with a selective advantage to survive primary chemotherapy.

Detection of CD22ΔE12 in PLC obtained from therapy-refractory pediatric B-lineage ALL patients implicates this genetic defect in the aggressive biology of relapsed or therapy-refractory leukemia in pediatric ALL patients. In addition, the detection of CD22ΔE12 mRNA species in 12 of 12 newly diagnosed infant ALL cases as disclosed herein, confirms the presence of CD22ΔE12 in 4 of 6 newly diagnosed infant ALL cases.

The CD22ΔE12 mutant provides a useful biomarker, for example in the evaluation of remission bone marrow samples for the presence of residual therapy-refractory clones, as a prognostic molecular marker to identify patients at high risk for treatment failure, and many additional diagnostic, prognostic, and therapeutic evaluations. In one example, detection of CD22ΔE12 mRNA in live leukemia cells can be performed, for example, using fluorescent oligonucleotide probes or dual-labeled stem-loop oligonucleotide hairpin probes, commonly known as “molecular beacons” in combination with linear fluorescence resonance energy transfer (FRET)-based, as described, for example, in Bao et al., 2009.

The observed aggressive biological behavior and signature transcriptome associated with CD22ΔE12 expression in relapsed ALL are not the result of a homozygous loss of function mutation. While the intronic mutations associated with CD22ΔE12 likely promote aberrant splicing by causing marked changes in the predicted secondary structures of CD22 pre-mRNA harboring target motifs for the hnRNP family splicing factors, there do not appear to be any exon deletions, and normal CD22 mRNA with corresponding intact CD22 protein is expressed along with Exon 12-deleted mRNA species with corresponding truncated CD22 protein in most cases with CD22ΔE12⁺ leukemia.

Intronic sequences often dictate the correct splicing of pre-mRNA and pathogenic intronic mutations, including single point mutations, have been linked to aberrant splicing and human disease. RNA splicing requires a complex interplay of multiple RNA-binding proteins that are equipped with domains to bind sequence motifs on single stranded RNA to ensure accurate determination of exon recognition. CD22ΔE12 is the first genetic defect implicating intronic mutations in the pathogenesis and biology of aggressive ALL. The observed impact of the recurring intronic mutations on the predicted secondary structures of the patients' CD22 pre-mRNA molecules support the hypothesis that these mutations would likely affect the recognition of 5′ splice site of exon 12 by the splicing machinery and perturb proper splicesome assembly thereby causing aberrant pre-mRNA splicing as the underlying mechanism of CD22ΔE12.

Recent discoveries regarding the molecular regulatory mechanisms governing RNA processing, alternative splicing, and pleiotropic functional profiles of splicing proteins may provide the foundation for therapeutic innovation against difficult to treat diseases associated with aberrant RNA processing or inappropriately amplified expression of specific gene products. For example, spliceosome mediated RNA transsplicing can potentially replace an aberrant transcript with a wildtype sequence (Buratti et al., 2010). The trans-splicing process is induced by engineered ‘RNA trans-splicing molecules’ (RTMs), which target a selected pre-mRNA to be reprogrammed via two complementary binding domains.

The unique ability of U1snRNP bound to the last exon to promote mRNA degradation can be used to design gene silencing strategies using synthetic bifunctional oligonucleotides known as U1 adaptors that contain a target domain complementary to a site in the target gene's last exon and a U1 domain for recruitment of U1snRNP via base-pair interaction with the U1 small nuclear RNA component (Buratti et al., 2010; Goraczniak et al., 2009). It has been shown that tethering of U1 snRNP to the target pre-mRNA using U1 adaptors inhibits poly(A)-tail addition and promotes degradation (Goraczniak et al., 2009).

CD22ΔE12 can act as a dominant-negative isoform by binding to the same cis ligands of CD22 and its abundance may prevent wild-type CD22 from in cis ligand binding leading to increased proliferation and defective apoptosis. It will be important to explore in future laboratory studies if the depletion of CD22ΔE12 with an anti-sense oligo via preventing the faithful translation of the exon 12-depleted aberrant CD22 mRNA species would change the biologic features of CD22ΔE12+ ALL blast cells and thereby abrogate the putative dominant-negative effects of the truncated CD22 product. Advances in the nanotechnology field provide a unique opportunity to selectively deliver appropriately designed antisense oligonucleotides or chimeric molecules for exon-specific splicing enhancement to therapy refractory leukemia cells by using multifunctional nanoparticles.

See, for example, Bao G, Rhee W J, Tsourkas A. Fluorescent probes for live-cell RNA detection. Annu Rev Biomed Eng 11:25-47, 2009; Buratti E, Baralle D. Novel roles of U1 snRNP in alternative splicing regulation. RNA Biology 7:412-419, 2010; and Goraczniak R, Behlke M A, Gunderson S I. Gene silencing by synthetic U1 adaptors. Nature Biotechnology doi:10.1038/nbt.1525, 2009.

The invention provided herein is based, in part, on the identification of mutations in CD22 in infant BCL. Provided herein are CD22ΔE12 polynucleotides and polypeptides encoded by such polynucleotides. In addition, methods of identifying patients with a B-cell disorder (e.g., BPL or hairy cell leukemia (HCL)) at risk of being resistant to anti-leukemia treatment targeted to CD22 are provided. Further provided are methods of treating a patient with a B-cell disorder based on the expression, or lack thereof, of a CD22ΔE12 polynucleotide and/or polypeptide in leukemic cells. Methods for predicting the level of severity of a B-cell disorder of a patient are also provided. Methods of treating leukemic cells expressing the mutated CD22ΔE12 polynucleotides described herein with targeted antibodies, toxins, immunconjugates, sense and antisense oligonucleotides, including morphilinos and microRNA molecules and the like to dampen or deplete CD22ΔE12 polynucleotides and/or induce apoptosis of CD22ΔE12 expressing cells.

CD22 is an inhibitory co-receptor of B-cells and B-cell precursors that acts as a negative regulator of multiple signal transduction pathways functional in B-cell homeostasis, survival, activation, and differentiation. The inhibitory and apoptosis-promoting signaling function of CD22 is dependent on recruitment of the Src homology 2 domain-containing tyrosine phosphatase (SHP)-1 to the immunoreceptor tyrosine-based inhibitory motifs (ITIMs) of its cytoplasmic domain upon phosphorylation by the Src family tyrosine kinase LYN (Songyang et al. (1993), Cell 72: 767-78; Law C L et al. (1996), J Exp Med 183: 547-60; Tuscano, et al. (1996), Eur. J. Immunol. 26: 1246-52; Cornall et al. (1998), Immunity 8: 497-508; Blasioli et al. (1999), J. Biol. Chem. 274: 2303-2307.).

Provided herein are CD22ΔE12 polynucleotides (e.g., DNA or RNA). In some embodiments, the provided polynucleotides have mutations as compared to a wild type CD22, e.g., human CD22 (SEQ ID NO:1) that affect splicing. The effects on splicing can include removal of all or a part of CD22 exon 12 following pre-mRNA processing. Mutations that can affect splicing of human CD22 exon 12 include mutations in GenBank Accession No. NC_{—}000019.9 as detailed in Table 1. In some cases, a CD22ΔE12 polynucleotide can include more than one mutation provided in Table 1. In some embodiments, a CD22ΔE12 polynucleotide can include one or more of the mutations provided in Table 1 as well as CD22 exon 12.

TABLE 1
Mutations that affect CD22 exon 12 splicing
Position of DNA Sequence Change

| | Standard Human Genome |
|---|---|
| Chr. Location in | Variation Society (HGVS) |
| NC_000019.9 | Nomenclature |
| g.35,836,751 | c.2327 + 128A > G |
| g.35,836,770 | c.2327 + 147G > A |
| g.35,836,826 | c.2327 + 203C > G |
| g.35,836,859 | c.2328 − 195A > G |
| g.35,836,747 | c.2327 + 124delT |
| g.35,836,763 | c.2327 + 140G > C |
| g.35,836,768 | c.2327 + 145delT |
| g.35,836,769 | c.2327 + 146delG |
| g.35,836,770 | c.2327 + 147G > C |
| g.35,836,808 | c.2327 + 185delA |
| g.35,836,855 | c.2328 − 199C > G |
| g.35,836,859 | c.2328 − 195A > G |
| g.35,836,727 | c.2327 + 104_105InsG |
| g.35,836,736 | c.2327 + 113C > A |
| g.35,836,764 | c.2327 + 141delC |
| g.35,836,808 | c.2327 + 185delA |
| g.35,836,726 | c.2327 + 103C > G |
| g.35,836,859 | c.2328 − 195A > G |

In some embodiments, a CD22ΔE12 polynucleotide can exclude all or part of the CD22 exon 12 without or without including any mutations that affect splicing. For example, a CD22ΔE12 polynucleotide can be an mRNA that excludes the CD22 exon 12(SEQ ID NO:2).

In some embodiments, the provided CD22ΔE12 polynucleotides can be included in vector, such as an expression vector or a vector for transgene insertion. An expression vector can be used, for example, to produce a CD22ΔE12 polypeptide in a cell or to produce a transgenic animal. In some embodiments, an expression vector comprising a CD22ΔE12 polynucleotide can be used to “knock in” a CD22ΔE12 polynucleotide at a specific location in a chromosome, such as the CD22 locus. Thus, a transgenic animal that has an exogenous CD22ΔE12 polynucleotide incorporated in to its genome by either targeted insertion or random insertion can be produced using a vector comprising a CD22ΔE12 polynucleotide.

An expression vector can include nucleic acid sequences other than a CD22ΔE12 polynucleotide sequence. Such nucleic acid sequences include a promoter suitable for promoting the expression of a CD22ΔE12 mRNA in a cell, a selection marker, or a sequence encoding a detection marker (e.g., GFP, myc tag, FLAG tag, poly-His tag, or RFP). Sequences such as promoters and enhancers are operatively linked to a CD22ΔE12 polynucleotide sequence in order to promote or enhance CD22ΔE12 polynucleotide expression, respectively. Sequences such as detection markers are operatively linked to a CD22ΔE12 polynucleotide sequence in order to produce a chimeric protein comprising a CD22ΔE12 polypeptide and the detection marker.

### Diagnostic Methods

Nucleic acid molecules including probes and primers can be used to identify a CD22ΔE12 polynucleotide, such as a CD22ΔE12 polynucleotide having a mutation described in Table 1. For example, a nucleic acid provided herein can be a probe that specifically hybridizes to a CD22ΔE12 polynucleotide. In another embodiment, a nucleic acid provided herein can be a primer designed to amplify a region of CD22 to identify the presence of one or more mutation that results in a CD22ΔE12 polynucleotide. The nucleic acids provided herein can be used in methods of identifying a CD22ΔE12 polynucleotide. For example, the nucleic acids provided herein can be used to identify whether a CD22 sequence has a mutation that alters exon 12 splicing. Methods for identifying a CD22ΔE12 polynucleotide include single-nucleotide polymorphism (SNP) analysis, PCR, RT-PCR, sequence analysis, and the like.

A mutation found in a CD22ΔE12 polynucleotide can result in reduced expression of a CD22 polypeptide and/or the expression of a CD22 polypeptide that is mutated from wild type CD22 (SEQ ID NO:1). Such mutated CD22 polypeptides are also provided herein. In some embodiments, the polypeptides provided herein lack all or part of the amino acid sequence encoded by CD22 exon 12. In some embodiments, the polypeptides provided herein include a sequence that is not included in wild type CD22. An example of such a sequence is SEQ ID NO:3 (RCRVLRDAETSPGLR). The presence of SEQ ID NO:3 can be indicative of a CD22ΔE12 mutation.

In some embodiments, an antibody (e.g., polyclonal or monoclonal) to SEQ ID NO:3 can be produced using known methods. For example, an expression vector comprising a CD22ΔE12 polynucleotide can be transformed into a bacteria and recombinant CD22ΔE12 polypeptide can be extracted from the transformed bacteria. The extracted protein can be introduced into, for example, a rabbit to produce a polyclonal antibody. Anti-CD22ΔE12 antibodies can be modified using known methods to produce, for example, humanized antibodies. Anti-CD22ΔE12 antibodies are useful, for example, for the detection of a CD22ΔE12 polypeptide.

In some embodiments, the provided polynucleotides and/or polypeptides can be used to identify individuals at risk of developing B-cell disorder (e.g., BPL or HCL). Risk of developing B-cell disorder can be predicted by determining whether a tissue or a cell (e.g., a lymphocyte) in an individual includes a CD22ΔE12 polynucleotide or polypeptide, and predicting, based on the presence or absence of the CD22ΔE12 polynucleotide or polypeptide, whether the individual is at risk of developing B-cell disorder. Specifically, the presence of a CD22ΔE12 polynucleotide or polypeptide in a tissue or cell of an individual is predictive of an increased risk of developing B-cell disorder as compared to an individual that lacks a CD22ΔE12 polynucleotide or polypeptide, and predicting, based on the presence or absence of the CD22ΔE12 polynucleotide or polypeptide.

The presence of a CD22ΔE12 polynucleotide can be detected by known methods. For example, genomic DNA from a region surrounding exon 12 of CD22 using PCR and sequenced to determine whether a mutation that affects the splicing of exon 12 is found in that region. In another example, single nucleotide polymorphism (SNP) analysis can be performed to identify whether a CD22ΔE12 polynucleotide is present in an individual's genome. In some embodiments, the presence of a CD22ΔE12 polynucleotide can be determined by analyzing RNA. For example, the presence of a CD22ΔE12 polynucleotide can be detected by using RT-PCR to identify whether a CD22 mRNA is expressed that lacks a nucleic acid sequence from exon 12.

The presence of a CD22ΔE12 polypeptide can be detected using antibody methods. For example, the presence of a CD22ΔE12 polypeptide can be detected by using an antibody that specifically binds a mutated portion of the polypeptide, for example, an inserted sequence such as RCRVLRDAETSPGLR (SEQ ID NO:3). In another example, a CD22ΔE12 polypeptide can be determined by comparing the detection of a polypeptide using an antibody specific for the amino terminal portion of CD22 to the detection of a polypeptide using an antibody specific for an amino acid sequence encoded by exon 12 of CD22.

The presence of CD22ΔE12 mRNA species can be detected, for example, using RT-PCR, as well as using oligonucleotides complementary to the exon 11-13 junction found in these mutants. Nanotechnology methods can be used for delivery of such molecules for example, as reported for detecting breast cancer related gene defects not related to CD22.

In some embodiments, the provided polynucleotides and/or polypeptides can be used to predict the level of aggressiveness of a B-cell disorder. A B-cell disorder's aggressiveness can be measured by known means. For example, a more aggressive B-cell disorder can disseminate more quickly or be more resistant to anti-leukemia therapies than a less aggressive B-cell disorder. Aggressiveness of a B-cell disorder in an individual can be predicted by determining whether a tissue or a cell (e.g., a leukemia cell) in an individual includes a CD22ΔE12 polynucleotide or polypeptide, and predicting, based on the presence or absence of the CD22ΔE12 polynucleotide or polypeptide, whether the individual is at risk of developing an aggressive B-cell disorder. Specifically, the presence of a CD22ΔE12 polynucleotide or polypeptide in a tissue or cell of an individual is predictive of an increased risk of developing a more aggressive B-cell disorder as compared to an individual lacking a CD22ΔE12 polynucleotide or polypeptide.

In some embodiments, the provided polynucleotides and/or polypeptides can be used to predict whether B-cell disorder in a patient will be resistant to an anti-leukemia treatment such as one or more of Vincristine, Dexamethasone, Prednisolone, L-asparaginase, PEG-Asparaginase, Daunorubicin, Fludarabine, Cytarabine, Mitoxantrone, Vinorelbine, Cyclophosphamide, ionizing radiation, for example. Whether a B-cell disorder in a patient will be resistant to an anti-leukemia treatment can be predicted by determining whether a tissue or a cell (e.g., a leukemia cell) in an individual includes a CD22ΔE12 polynucleotide or polypeptide, and predicting, based on the presence or absence of the CD22ΔE12 polynucleotide or polypeptide, whether the B-cell disorder will be resistant to an anti-leukemia treatment. Specifically, the presence of a CD22ΔE12 polynucleotide or polypeptide in a tissue or cell of an individual having B-cell disorder is predictive of an increased risk of a B-cell disorder being more resistant to an anti-leukemia treatment as compared to an individual lacking a CD22ΔE12 polynucleotide or polypeptide.

In some embodiments, the presence of a CD22ΔE12 polynucleotide or polypeptide in a tissue or cell of an individual having B-cell disorder is predictive of resistance of the B-cell disorder to an anti-CD22 antibody that lacks fusion toxin or immunoconjugate. The presence of a CD22ΔE12 polynucleotide or polypeptide may still indicate that the B-cell disorder is sensitive to a treatment including an anti-CD22 antibody fusion toxin or immunoconjugate such as one or more of Epratuzumab, a humanized monoclonal antibody targeting CD22, anti-CD22 mAb (G5/44), a CD22-targeted innunoconjugate of CalichDMH designated CMC-544 (inotuzumab ozogamicin), and the like.

Thus, the presence of a CD22ΔE12 polynucleotide or polypeptide in a tissue or cell of an individual having B-cell disorder can be used to determine an appropriate anti-leukemia therapy for a patient having B-cell disorder. For example, the presence of a CD22ΔE12 polynucleotide or polypeptide in a tissue or cell of an individual having B-cell disorder would indicate that the use of a more aggressive anti-leukemia therapeutic regimen should be used or that an anti-CD22 immunotherapy that lacks a fusion toxin or immunoconjugate should be avoided. Conversely, the absence of a CD22ΔE12 polynucleotide or polypeptide in a tissue or cell of an individual having B-cell disorder would indicate that an anti-CD22 fusion toxin or immunoconjugate would be an appropriate therapy rather than an anti-CD22 immunotherapy that lacks a fusion toxin or immunoconjugate.

Diagnostic methods further include use of the gene signatures disclosed herein for the diagnosis of B-cell disease, including BLL, for identifying patients at risk for aggressive B-cell disease, for evaluating patient response to therapeutic treatments, and the like. The preferred gene signature for interrogation contains the following six gene markers whose reduced expression is associated with aggressive B-cell disease, as discussed more fully in the Examples below: APC, GNB2, MDM2, SATB1, CCNG1, and TP53. Additional genes include those set out in the genetic profile determined and described in the Examples below, and confidentially deposited with NCBI's GEO database and available for public access beginning Sep. 13, 2010.

### Therapeutic Methods

The present invention teaches the association of particular CD22 mutations associated with disease, particularly leukemia such as BPL and ALL. The presence of CD22ΔE12 mutations indicate the presence of disease, and provides a basis for targeted therapeutic treatments employing, for example, antibodies that specifically bind mutated CD22ΔE12, as discussed herein, as well as anti-CD22ΔE12 antibodies alone or fused to a cytotoxic, cytostatic, or other chemotherapeutic molecule, sense and antisense molecules directed to dampen or deplete CD22ΔE12 polynucleotides and/or induce apoptosis of CD22ΔE12 expressing cells, including, for example, morpholinos, shRNA, and microRNA molecules and the like. Therapeutic antibodies also include those directed against CD22 alone or in combination with other B-cell antigens such as CD19, CD20, and CD40. Such antibodies can be used to functionalize nanoparticles or oligonucleotides.

A morpholino useful as a therapeutic molecule include, for example, a 24-mer phosphorothioate oligo (S-oligo) representing the antisense orientation of the Exon 11-Exon 13 junction of the aberrant CD22ΔE12 mRNA (5′-GACTCTGCATCTCTTTTTATTCCT-3′) (SEQ ID NO: 4) having all diester bonds substituted to provide greater nuclease resistance. The anti-sense oligo will change the biologic features of CD22ΔE12+ leukemia cells by preventing the faithful translation of the exon 12-depleted aberrant CD22 mRNA species and thereby abrogating the dominant-negative effects of the truncated CD22 product.

It is well established that CD22 via its sialic acid binding domains is bound in cis to BCR as well as other surface membrane-associated co-receptors (e.g. CD45) in B-cells and B-cell precursors. These interactions are required for the normal inhibitory signaling function of CD22 and blocking them has been associated with reduced tyrosine phosphorylation of CD22 cytoplasmic domain and impaired recruitment of the inhibitory tyrosine phosphatase SHP-1 hyperactivation of B-cells with increased calcium signaling.

CD22ΔE12 can act as a dominant-negative isoform by binding to the same cis ligands of CD22 and its abundance may prevent wild-type CD22 from in cis ligand binding leading to development of a lymphoproliferative state with defective apoptosis.

Such therapeutic oligonucleotides may be delivered to the targeted genes using various known expression vectors such as lentiviral expression vectors and the like. Another preferred oligonucleotide delivery vehicles are nanoparticles adapted to carry CD22ΔE12 directed sense or antisense oligonucleotides, including shRNA, microRNA, and the like. The CD22ΔE12 directed molecules can be used in combination with standard chemotherapy or radiation therapies.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

The present invention is further exemplified by the following examples. The examples are not intended to in any way limit the scope of the present application and its uses.

### EXAMPLES

### Example 1

### Identification of a CD22 Mutation in B-Precursor Leukemia

### Methods and Materials—Leukemic Cells

Highly enriched populations of Ficoll-Hypaque-separated surplus leukemia cells isolated from bone marrow specimens of 6 infants (PT1-PT6) and 3 children (PT7-PT9) with newly diagnosed acute B-precursor leukemia (BPL) as well as surplus leukemia cells isolated from peripheral blood specimens of 3 adults with hairy cell leukemia (HCL) (PT10-PT12) were used in the described experiments with approval of the PHI Institutional Review Board (IRB) under the exemption category (45 CFR Part 46.101; Category #4: Existing Data, Records Review, and Secondary Use of Pathologic Specimens) in accordance with DHHS guidelines. Controls included (a) the Burkitt's/B-ALL leukemia/lymphoma cell lines RAJI (ATCC No. CCL-86), DAUDI (ATCC No. CCL-213), and RAMOS (ATCC No. CRL-1596), (b) B-cell precursor ALL cell lines REH (Pre-Pre-B, ATCC No. CRL-8286), NALM-6 (Pre-B, DSMZ No. ACC-128), normal fetal liver B-cell precursor cell lines FL8.2⁺ (CD2⁺CD19⁺ Pro-B/T) and FL8.2⁻ (CD2⁻CD19⁺ Pro-B) (1,2), and (c) the EBV-transformed B-lymphoblastoid cell line BCL-4 from a non-leukemic individual.

In addition, surplus leukemia cells isolated from bone marrow specimens of 31 infants (<1 year of age) with newly diagnosed ALL, who were treated on the CCG Infant ALL Protocol CCG-1953 and 23 children (>1 year of age) with newly diagnosed high risk ALL, who were treated on the CCG High Risk ALL Protocol CCG-1961 (Eligibility: Age≧10 years or Age 1-9 years with presenting WBC≧50,000/μL) as well as 7 children with newly diagnosed standard risk ALL, who were treated on the CCG Standard Risk ALL protocol CCG-1952 (Eligibility: Age 1-10 years and WBC<50,000/μL) were used for gene expression profiling (3) with approval of the PHI IRB under the exemption category (45 CFR Part 46.101; Category #4: Existing Data, Records Review, and Secondary Use of Pathologic Specimens) in accordance with DHHS guidelines. The secondary use of leukemic cells for subsequent molecular studies did not meet the definition of human subject research per 45 CFR 46.102 (d and f) since it does not include identifiable private information, as confirmed by the IRB (CCI) at Children's Hospital Los Angeles (CHLA).

### Western Blot Analysis of CD22 Expression

Western blot analysis of whole cell lysates for CD22 expression was performed by immunoblotting using N-20, a polyclonal goat IgG CD22 antibody recognizing the N-terminus of the human CD22 molecule (Santa Cruz, Catalog #7031), C-20, a C-terminal anti-CD22 antibody (Santa Cruz, Catalog #7029), and the ECL chemiluminescence detection system (Amersham Life Sciences).

### Apoptosis Assays

Leukemic cells from 3 infant BPL patients (PT1, PT5, PT6) with CD22ΔE12 as well as DAUDI Burkitt's leukemia and FL8.2⁻ normal fetal liver pro-B cells were treated with the anti-CD22 monoclonal antibody HB22.23 (6) at 1.0 and/or 10 μg/mL final concentrations. To detect apoptotic fragmentation of DNA, cells were harvested 24 hours after exposure to anti-CD22 antibodies. DNA was prepared from Triton-X-100 lysates for analysis of fragmentation (7,8). In brief, cells were lysed in hypotonic 10 mmol/L Tris-HCl (pH 7.4), 1 mmol/L EDTA, 0.2% Triton-X-100 detergent; and subsequently centrifuged at 11,000×g. To detect apoptosis-associated ladder-like DNA fragmentation, supernatants were electrophoresed on a 1.2% agarose gel, and the DNA fragments were visualized by ultraviolet light after staining with ethidium bromide.

### RT-PCR Analysis of CD22 Expression in Leukemia Cells

Reverse transcription (RT) and polymerase chain reaction (PCR) were used according to published PCR assay procedures (13) to amplify a 975-bp region (1858 bp to 2833 bp, GenBank accession code X59350) of the CD22 transcript. Total cellular RNA was extracted from cells lysed in guanidinium isothiocyanate using the RNeasy™ total RNA isolation kit (Qiagen, Santa Clarita, Calif.). cDNA was synthesized from total RNA using random primers and Superscript II reverse transcriptase (Gibco BRL). Oligonucleotide primers, 22-1 (SEQ ID NO: 5) (5′-GCCCGGGGGACCAAGTGATG-3′) and 22-2 (SEQ ID NO: 6) (5′-GTGGAAGAGAACAGGGGCAGGAGT-3′) were used to amplify the target PCR product encompassing the sequence corresponding to the transmembrane and intracellular domains of CD22 The enzyme mix eLONGase [Tag polymerase plus the proofreading (3′->5′ exonuclease activity) Pyrococcus species GB-D polymerase, Gibco BRL] was used with the following cycling conditions: 1 cycle (2 minutes 94° C., 1 minute 55° C., 1 minute 72° C.); 14 cycles (1 minute 94° C., 1 minute 55° C., 1 minute 72° C.); 19 cycles (1 minute 94° C., 1 minute 55° C., 3 min 72° C.); 1 cycle (1 minute 94° C., 1 minute 55° C., 7 minutes 72° C.). Negative controls included PCR products from an RNA-free cDNA synthesis and amplification reaction (negative control 1) and a DNA polymerase-free reaction (negative control 2).

PCR products were separated by electrophoresis in 1.2% agarose and visualized by ethidium bromide staining. In parallel, PCR products were transferred to nylon membranes and hybridized with an oligonucleotide probe specific for the CD22 Exon 11 sequence (5′-CCT GCC TCG CCA TCC TCA TCC-3′) (SEQ ID NO:7). The RT-PCR products were gel eluted (Geneclean II kit, Bio 101, Vista, Calif.) and then cloned by TA Cloning into PCR-2.1 (Invitrogen, San Diego, Calif.) for restriction analysis and subsequent sequencing. For EcoRI restriction digest analysis, the insert was released following digestion with EcoRI. Two EcoRI fragment sizes (608-bp and 367-bp) are expected for the insert based on GenBank Data (HSRNACD22). The insert was sequenced by cycle sequencing with Cy5-labeled primers and ThermoSequenase Fluorescent Labeled Primer Cycle Sequencing Kit (Amersham Pharmacia Biotech, Piscataway, N.J.) using an automated ALF express sequencer (Amersham Pharmacia Biotech) and analyzed using DNAStar LaserGene. The sequences were compared with the published human cDNA CD22 sequence (SEQ ID NO: 8) obtained through GenBank (Accession codes X59350 and U62631, NCBI Reference Sequence: NP_{—}001762.2).

### SCID Mouse Model of Infant BPL

In the SCID mouse xenograft experiments, female CB.17 SCID mice (6-8 weeks of age; Taconic/Germantown, N.Y.) were inoculated intravenously with 0.5 mL of a cell suspension containing 1×10⁶ primary infant BPL cells. All SCID mice were electively killed at 60 days unless they died or became moribund earlier due to their disseminated leukemia (SI-text). At the time of their death or killing, mice were necropsied to confirm leukemia-associated marked hepatomegaly and/or splenomegaly.

### Genomic PCR Analysis of CD22 Gene in Leukemia Cells

DNA sequencing was carried out using the BigDye Terminator v.3.1 cycle sequencing kit (Applied Biosystems, Foster City, Calif.) (FIGS. 2 to 4). Total genomic DNA was extracted from both patient's leukemia cells and cell lines using the Qiagen DNeasy Blood & Tissue kit (Catalog No 6950) according to the manufacturer's specifications. An 885-bp PCR product encompassing the CD22 exons 10, 11 and their exon-intron junctions was PCR-amplified using the genomic PCR primers CD22-10/11-seqF4 (5′-CACAGCTATACTGCCGTGAA-3′; SEQ ID NO:9 and CD22-10/11-seqR4 (5′-AGGCAGAGTCTCAGTATGTC; SEQ ID NO:10). The sequencing primer was CD22-10/11-seq (5′-GCTCCTTCAAGGAGAATTAGTG-3′; SEQ ID NO:11). A 905-bp PCR product encompassing the CD22 exons 12, 13, and their exon-intron junctions was PCR-amplified using the genomic PCR primers CD22-seqF10 (5′-GGCATGAGGCAGACTGTGAA-3′; SEQ ID NO:12) and CD22-seqR10 (5′-AACCTCTGCCACCACGGATG-3′; SEQ ID NO:13). The sequencing primers were CD22-12/13-seq (5′-CCACTCGGCAACAAGCCTCT-3′; SEQ ID NO:14) and CD22-12-seqr (5′-GAAGGAGCAGGTCCACTTCT; SEQ ID NO:15). CD22 exon was PCR amplified using the genomic PCR primers CD22-seqF11N (5′-CACAGCCAGTTTCCTGACAC-3; SEQ ID NO:16) and CD22-R11 (5′-AGGGACCCTGGCAGCATCTGAGAGCAAAAGTTCTTTGAAGTGGCATCTGA-3′; SEQ ID NO:17).

The primer sets (50 pmol/μL) (0.7 μL of each primer, 50 μL reaction volume, 150 ng genomic DNA, 0.5 μL of 10 mM dNTP, 2.5 UTaq polymerase/Invitrogen-Cat. No. 12355-036) were used with the following thermal cycling conditions: 1 cycle for initial denature (5 minutes 95° C.), 32 cycles (30 seconds 95° C., 30 seconds 58° C., 1 minute 72° C.); hold at 72° C. for 5 minutes; indefinite hold at 4° C. The PCR products were directly sequenced using the indicated PCR primers in 5 μL reaction volumes containing 0.5 μL BigDye terminator mix v3.1, 1 μL 5× sequencing buffer (Applied Biosystems), 1 μL 3.2 pmol primer, and 25 ng PCR product. Sequencing thermal cycling parameters were: 1 cycle (1 minute, 96° C.), 35 cycles (10 seconds at 96° C., 5 seconds at 50° C., 150 seconds at 60° C.); hold 180 seconds at 60° C., and indefinite hold at 4° C. The sequencing products from each reaction were cleaned using GenScript QuickClean 5M purification kit (GenScript, MD) and analyzed on an ABI 3730XL DNA Analyzer (Applied Biosystems). Sequence obtained from the genomic PCR products was analyzed using SeqMan II contiguous alignment software in the LaserGene suite from DNASTAR Inc. and the MegAlign multi-sequence alignment software in comparison with the wild-type CD22 sequence (NCBI Reference Sequence: NC_{—}000019.9. Homo sapiens chromosome 19, Genome Reference Consortium Human Build 37/GRCh37) primary reference assembly, on www at ncbi.nih.gov.

### Determination of the Pre-mRNA Secondary Structure in Intron-Derived Segments

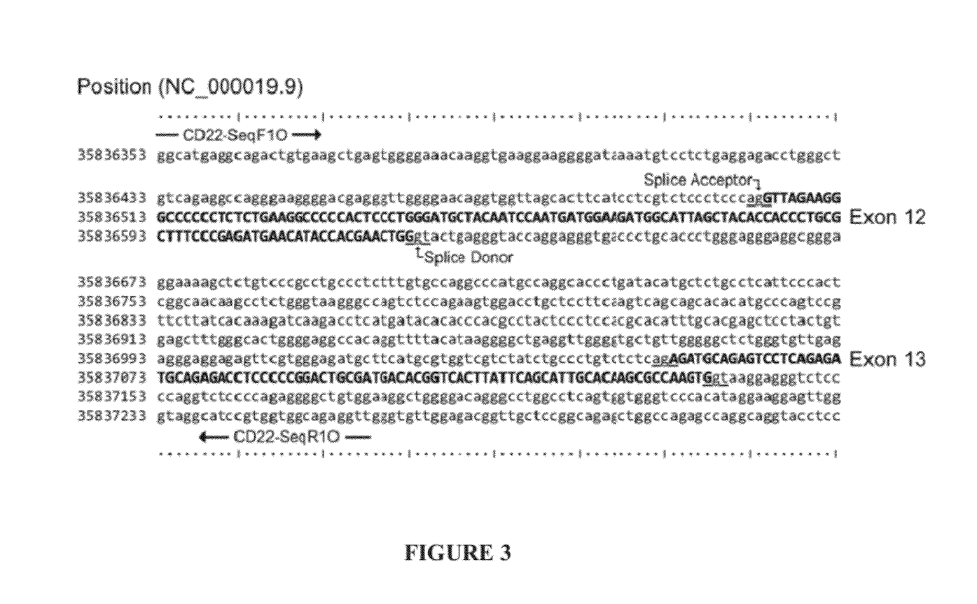

The CD22 sequence was interrogated using the UCSC Genome browser (http://genome.ucsc.edu/) that reported and aligned known human ESTs in the CD22 region of interest (chr19: 35,836,500-35,837,143). The splice acceptor and donor sites were deduced from this alignment and cross-referencing the Collaborative Consensus Coding Sequence (CCDS) project (FIG. 3). The assigned CCDid number ensures that coding sequences are consistently represented on the NCBI, Ensemb1, and UCSC Genome Browsers (hg19_ccds Gene_CCDS12457.1 for CD22). The DNA sequence for wild type and patient sequences were converted to positive strand RNA complement sequences for alignment using the Clust W program (BioEdit Sequence Alignment editor).

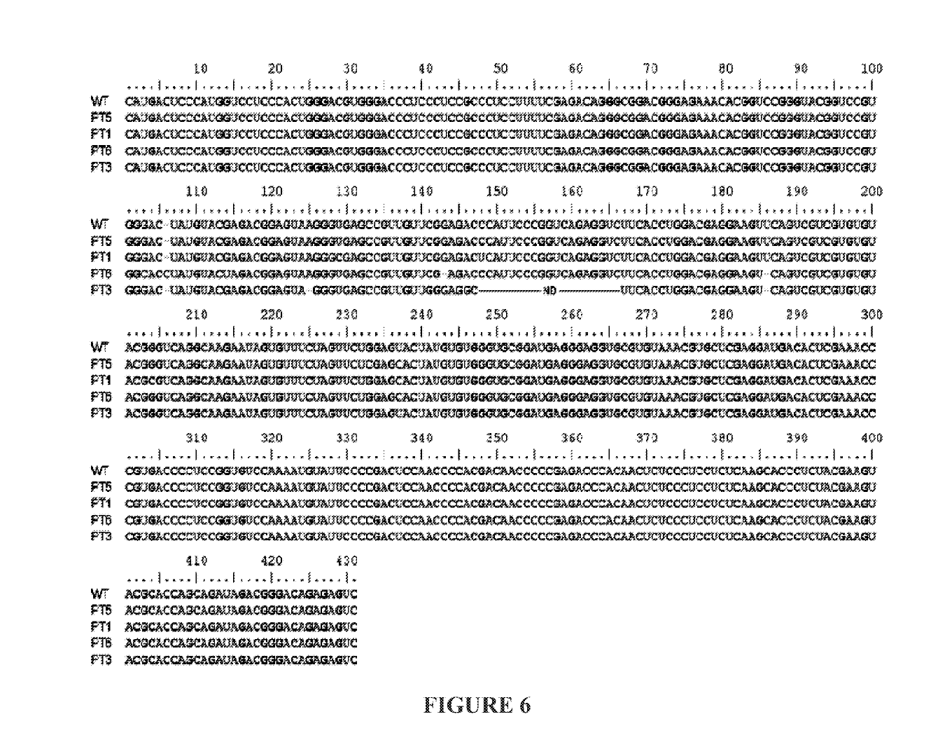

Multiple alignment was constructed using gap penalties in a position- and residue-specific manner such that all pairs of sequences were aligned separately in order to calculate a distance matrix for each pair of sequences (Fast Approximate Method), then a guide tree was calculated from the distance matrix and the sequences were progressively aligned according to the branching order in the guide tree (Neighbour-Joining method) (FIG. 6). The accessibility of target motifs to RNA binding proteins was assessed after predicting of secondary structure of the positive strand of the CD22 pre-mRNA molecule using the minimum free energy (MFE) calculations for sequences obtained from intronic regions between exons 12 and 13 of the CD22 genomic sequence (NC_{—}000019.9; 35,836,624-35,837,053, Homo sapiens chromosome 19, GRCh37 primary reference assembly). We implemented a dynamic programming algorithm, RNAfold, provided by the Vienna RNA package (http://rna.tbi.univie.ac.at/) that calculated MFE folding and equilibrium base-pairing probabilities for the pre-mRNA segment corresponding to the intronic RNA complement (C^{35,836,624}-C^{35,837,053}) to explore how the target sequences for RNA binding proteins residing in loop structures, bulges or binding pair probabilities with low values varied between the wild-type and patient secondary structures.

### Results

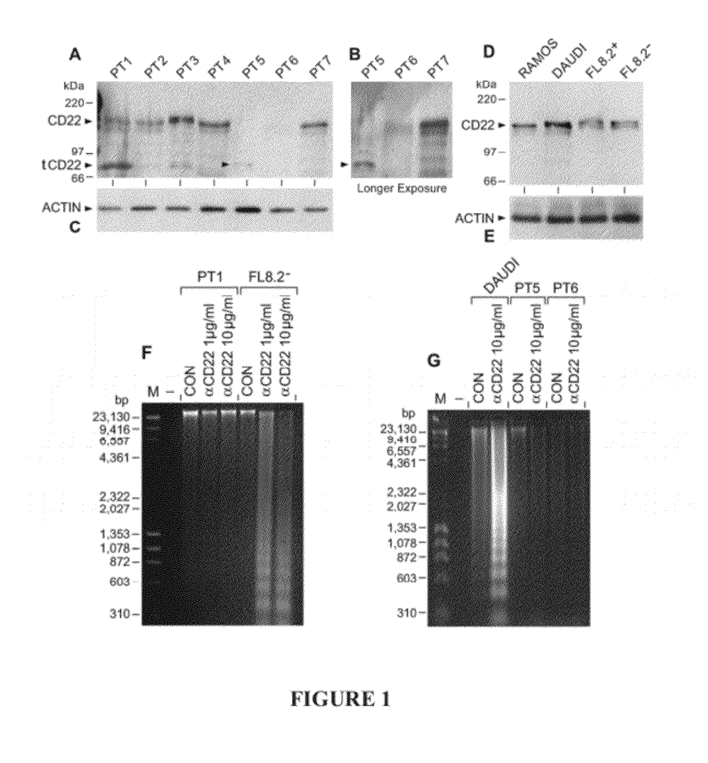

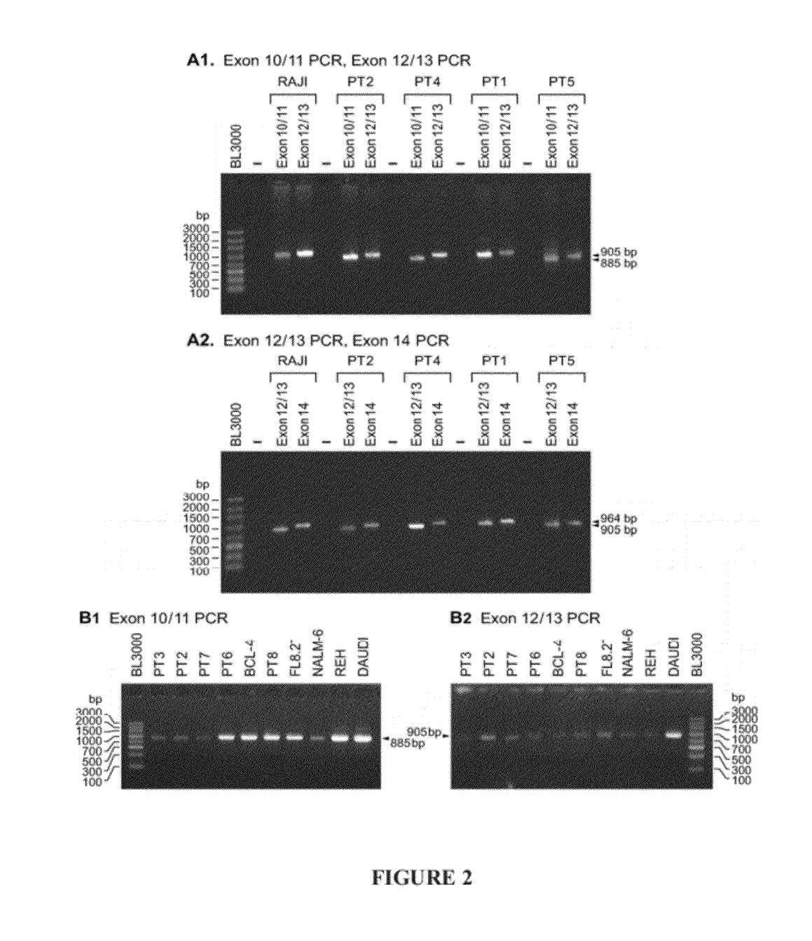

Primary leukemic cells from infants with BPL were evaluated for possible structural and functional CD22 defects. Using Western blot analyses, a truncated CD22 protein in primary leukemic B-cell precursors from infant patients with newly diagnosed BPL was detected (FIG. 1, A-C), that was not present in fetal-liver derived normal B-cell precursors or Burkitt's leukemia/lymphoma cell lines (FIGS. 1, D and E). While a 140 kDa intact CD22 co-receptor protein was detected in all control cell lines and primary leukemic cells from a pediatric BPL patient, no or very low levels of intact CD22 could be detected in the lysates of leukemic cells from some of the infant BPL patients (FIG. 1, A-E).

To test whether the truncated CD22 co-receptor of infant BPL cells can transmit apoptotic signals, the effects of CD22 ligation on infant BPL cells as compared to control cell lines using the apoptosis-inducing HB22.7 monoclonal antibody that blocks the ligand binding site of CD22 were evaluated. HB22.7 induced apoptosis in DAUDI and FL8.2⁻ cell lines that express an intact CD22 protein, but not in infant BPL cells from PT1 and PT5 expressing a truncated CD22 or PT6 with negligible levels of CD22 (FIGS. 1, F and G). Thus, the truncated CD22 co-receptor that is selectively expressed in infant BPL cells is functionally defective.

To determine if the abnormal CD22 protein expression profile affects the in vivo biological behavior of infant BPL cells, primary leukemic cells from PT1, PT3, PT5, expressing a truncated CD22 and PT6, expressing very low levels of intact CD22 were compared to primary leukemic cells from PT2, and PT4, expressing abundant levels of an intact CD22 in the absence of truncated CD22 were compared for their ability to cause disseminated leukemia in a SCID mouse xenograft model of infant leukemia. While 38 of 40 SCID mice inoculated with BPL cells exhibiting an abnormal CD22 expression profile developed disseminated leukemia within 60 days, none of the 20 mice inoculated with BPL cells with a normal CD22 expression profile did (Table 2, Fishers Exact test, P<0.0001). These results suggest that the expression of a truncated CD22 protein devoid of pro-apoptotic function may provide infant BPL cells with an in vivo growth and survival advantage.

TABLE 2
In Vivo Growth of Infant BPL Cells in SCID Mice in relationship
to Expression of a Truncated CD22 Co-Receptor Protein

| | Patient | | | | | |
|---|---|---|---|---|---|---|
| | PT1 | PT2 | PT3 | PT4 | PT5 | PT6 |
| Age/sex | 4/F | 8/M | 6/M | 2/F | 2/F | 1/F |
| MLL-AF4 Status | + | + | − | + | + | + |
| Truncated CD22 expression | + | − | + | − | + | + |
| Overt Leukemia SCID Mice | 10/10 | 0/10 | 8/10 | 0/10 | 10/10 | 10/10 |

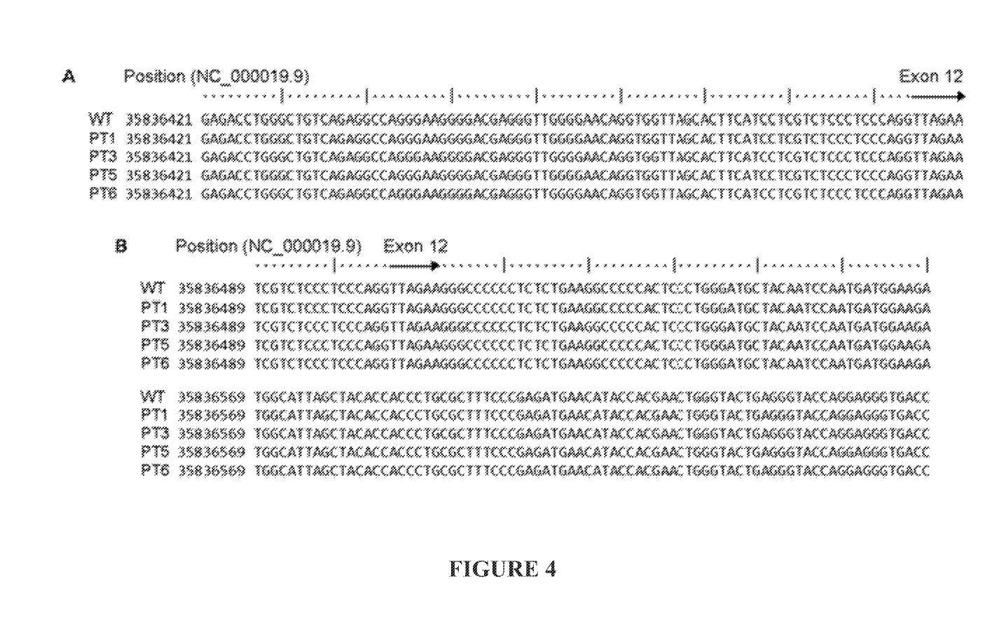

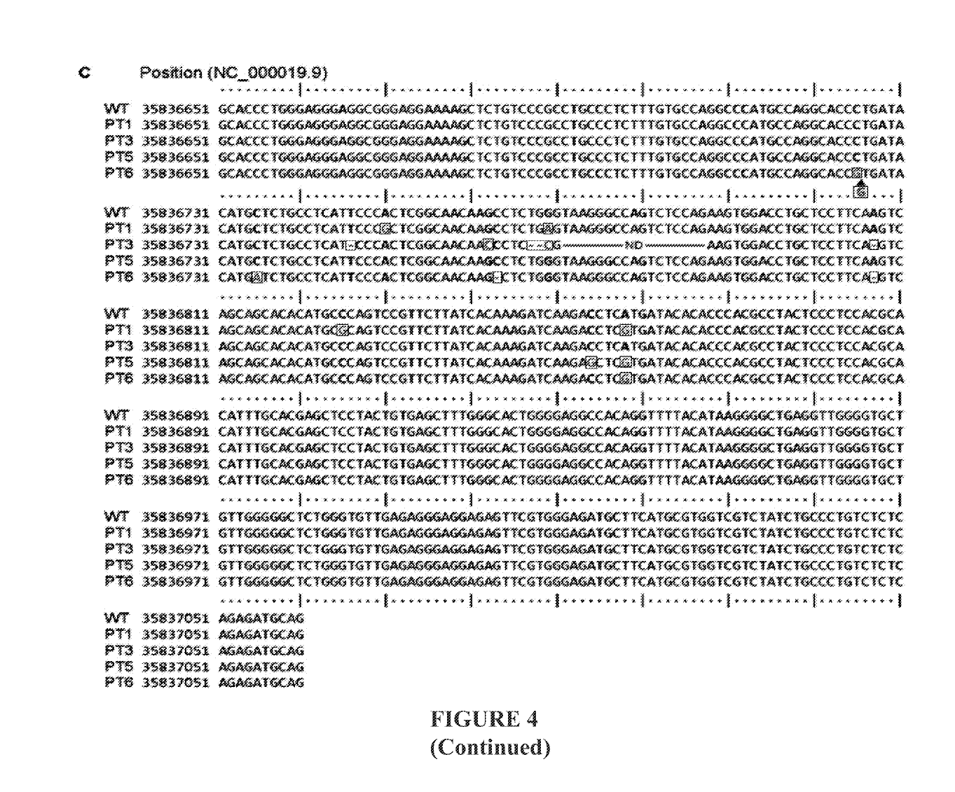

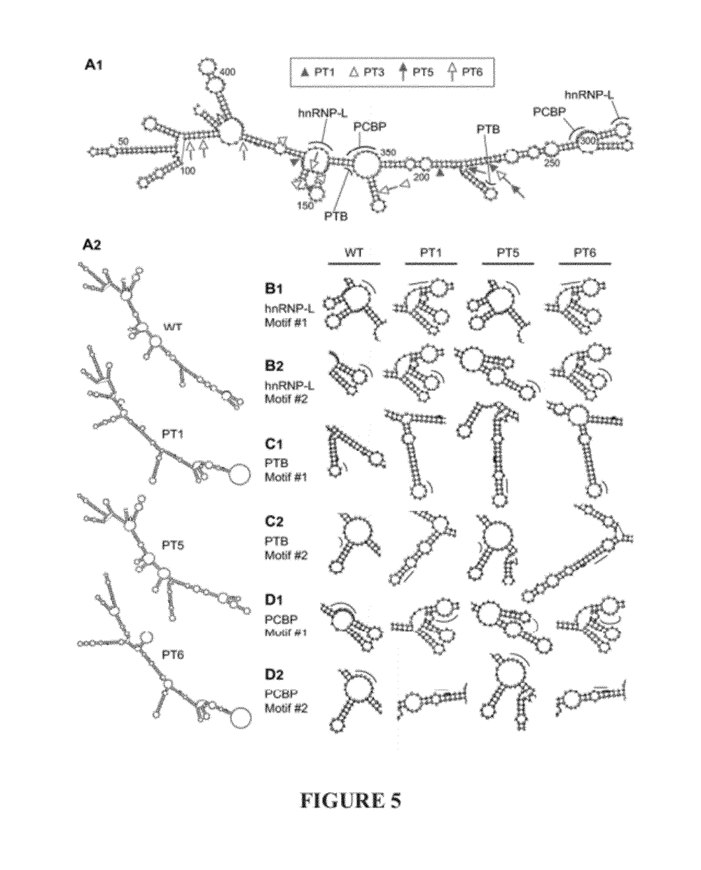

To explore the genetic mechanism for the expression of a structurally and functionally defective CD22 co-receptor protein in infant BPL cells, exons 10-14, encoding the signal transmitting transmembrane and cytoplasmic domains, were amplified and sequenced by PCR from genomic DNA samples from primary leukemia cells of 6 infants with newly diagnosed BPL. Normal size PCR products were obtained in each of the 6 infant BPL cases, including those with truncated or near absent CD22 co-receptor protein expression, providing evidence against genomic deletions of CD22 exons encoding the cytoplasmic and transmembrane domains as a cause for the observed expression of a truncated CD22 protein or substantially reduced expression levels of an intact protein (FIG. 2). However, multiple homozygous mutations within a 132-bp segment of the intronic sequence between exons 12 and 13 (NC_{—}000019.9: c.2327+104/G^{[35,836,727]}-c.2328-195/G^{[35,836,859]}) were identified, including transversions/transitions, deletions, and insertions (FIG. 3 and FIG. 4, Table 3). Surprisingly, none of the mutations were within exon 12 or its splice acceptor/donor sites or in the intronic sequence between exons 11 and 12.

The genomic C D22 intron12 gene sequences isolated from the four individual infant BPL patients (PT1, PT3, PT5, PT6) were confidentially deposited in both EMBL and GENBANC databases and will be publically available on line on Sep. 13, 2010 under the following accession numbers for the respective samples PT1, PT3, PT5, PT6:

EMBL Accession No: FR687955, FR687956, FR687957, FR687958

GenBanc Accession No: HQ225617, HQ225618, HQ225619, HQ225620

Genomic mutations discovered in the infant BPL patients were next cross-referenced with the database of single nucleotide polymorphisms (SNP) housed in NCBI's Entrez system of data mining tools (dbSNP; www.ncbi.nlm.nih.gov/sites/entrez?db=snp). The intronic region of interest between exons 12 and 13 of the CD22 gene (forward mRNA strand at NM_{—}001771) was queried and found that it contains 5 SNPs of unknown clinical or biological significance, namely rs4805119, rs10406539, rs10413500, rs10413526, and rs4805120. Notably, 4 of 4 mutations in PT1, 2 of 2 mutations in PT5 and 2 of 6 mutations in PT6 were at the exact locations of these previously reported SNPs (Table 3). Four of the 6 mutations in PT6 and all 6 mutations in PT3 were unique and showed no concordance with reported SNP sites.

TABLE 3
Homozygous Intronic Mutations of CD22 Gene in Infant BPL Cells

| | | Position of DNA Sequence Change | | |
|---|---|---|---|---|
| | | | Standard Human | |
| | | | Genome | |
| | | Chr. Location | Variation Society | Corre- |
| | Mutation | in | (HGVS) | sponding |
| Patient | No. | NC_000019.9 | Nomenclature | SNP |
| PT1 | 1 | g.35,836,751 | c.2327 + 128A > G | rs4805119 |
| | 2 | g.35,836,770 | c.2327 + 147G > A | rs10406539 |
| | 3 | g.35,836,826 | c.2327 + 203C > G | rs10413500 |
| | 4 | g.35,836,859 | c.2328 − 195A > G | rs4805120 |
| PT3 | 1 | g.35,836,747 | c.2327 + 124delT | — |
| | 2 | g.35,836,763 | c.2327 + 140G > C | — |
| | 3 | g.35,836,768 | c.2327 + 145delT | — |
| | 4 | g.35,836,769 | c.2327 + 146delG | — |
| | 5 | g.35,836,770 | c.2327 + 147G > C | — |
| | 6 | g.35,836,808 | c.2327 + 185delA | — |
| PT5 | 1 | g.35,836,855 | c.2328 − 199C > G | rs10413526 |
| | 2 | g.35,836,859 | c.2328 − 195A > G | rs4805120 |
| PT6 | 1 | g.35,836,727 | c.2327 + | — |
| | | | 104_105InsG | |
| | 2 | g.35,836,736 | c.2327 + 113C > A | — |
| | 3 | g.35,836,764 | c.2327 + 141delC | — |
| | 4 | g.35,836,808 | c.2327 + 185delA | — |
| | 5 | g.35,836,826 | c.2327 + 203C > G | rs10413500 |
| | 6 | g.35,836,859 | c.2328 − 195A > G | rs4805120 |

Intronic sequences often dictate the correct splicing of pre-mRNA and pathogenic intronic mutations, including single point mutations, have been linked to aberrant splicing and human disease (Hatta et al. (1999), Immunogenetics 49:280-286; The Wellcome Trust Case Control Consortium, Nature 447:661-678 (2007); Kawamata et al. (2008), Blood 111:776-784; Miyagawa et al. (2008), Rheumatology 47(2):158-64; Mullighan et al. Leukemia 23:1209-1218). RNA splicing requires a complex interplay of multiple RNA-binding proteins that are equipped with domains to bind sequence motifs on single stranded RNA to ensure accurate determination of exon recognition.

In silico interrogation of the wild-type CD22 pre-mRNA segment derived from the 132-bp intronic sequence between exons 12 and 13 for accessible splicing factor binding sites resulted in identification of multiple potential binding sites for the RNA-binding proteins hnRNP-E2/PCBP, hnRNP-I/PTB, and hnRNP-L, three members of the heterogenous nuclear ribonucleoprotein family of splicing factors (See, for example, Venables et al. (2004), Cancer Res. 64, 7647-7654; Hui et al. (2005), EMBO Journal 24, 1988-1998; Baralle et al. (2005), J. Med. Genet. 42:737-748; Venables et al. (2008), Mol Cell Biol 28, 6033-6043; Hung et al. (2008), RNA 14, 284-296; Z. Wang et al. (2008), RNA 14, 802-813; Pomares et al. (2009), IVOS 50, 5107-5114 (2009); Davis et al (2009), Hum Mutat 30, 221-227; Fogel et al. (2009), Cerebellum 8, 448-453; Galante et al. (2009), RNA Biology 6:426-433) (FIG. 5A.1).

A computational secondary structure prediction algorithm was used to predict the effect the observed mutations might have on the secondary structure of the pre-mRNA and the accessibility of its target motifs for splicing factors. Sequence alignment of the pre-mRNA sequences of infant BPL patients with the consensus pre-mRNA sequence yielded only few differences (FIG. 6).

The RNA sequence deviations from the wild-type forward strand complement RNA sequence (C^{35,836,624}-C^{35,837,53}) for the depicted pre-mRNA segment are as follows: PT1: 129 U>C, 148 C>U, 204 G>C, 237U>C; PT3: 125A>-, 141C>G, 146A>G, 186U>-; PT5:233 G>C, 237U>C; PT6: 103G>C, 106->C, 114G>U, 143G>-, 186U>-, 237U>C. Residues corresponding to positions 148 to 166 in the aligned sequence could not be accurately determined for PT3. However, the documented mutations resulted in strikingly different secondary structure predictions (FIG. 5A.2). In particular, there were marked changes in secondary structure conformation and folding patterns that affected the target motifs for hnRNP-E2/PCBP, hnRNP-I/PTB, and hnRNP-L as well as the surrounding structural features in the predicted pre-mRNA molecules (FIG. 5, B-D).

Whereas the wild-type secondary structure contained 11 hairpin loops, 2 bulges, 6 multi-branched loops and 8 internal loops in the RNA helix, there were 9 hairpin loops, 4 bulges, 5 multi-branched loops and 11 internal loops in PT1 secondary structure; 10 hairpin loops, 3 bulges, 6 multi-branched loops and 10 internal loops in PT5 secondary structure, and 7 hairpin loops, 3 bulges, 5 multi-branched loops and 14 internal loops in PT6 structure. (FIGS. 5, B.1 & B.2).

The CACA binding motif for hnRNP-L appeared at a base of a multi-branched loop comprised of two hairpin loops in wild-type and PT5 pre-mRNA, while this motif was sequestered between an internal loop and a multi-branched loop through formation of a double strand between CAC and GUG complementary pairs in pre-mRNA from PT1 and PT6. A second loop structure with an ACAC binding motif showed open access in a hairpin loop structure from wild-type and PT5 pre-mRNA and apparent potential for steric hindrance adjacent to a region with an internal loop and a bulge in pre-mRNA from PT1 and PT6. (FIG. 5, C.1 & C.2) A PTB-binding site UCU showed two bases in a hairpin loop structure of wild-type pre-mRNA. Notably, in PT1 and PT6 all three bases appear within the hairpin loop at the end of a stem with 10 base pairs making this motif more accessible for PTB binding.

An alternative PTP-binding site (viz.: CCU) formed a junction between a multi-branched loop and a stem in wild-type pre-mRNA and PT5 pre-mRNA, but in the other two patients the GGG is double-stranded making this motif inaccessible to protein binding. (FIGS. 5, D.1 and D.2) There were two binding sites for PCBP that exhibited variation in the binding site accessibility and surrounding structural conformations for the wild-type and patient sequences.

In one motif, the multi-branched portion of the double hairpin loop structure contained a triple C site and the junction with the helix contained the quadruple C site with low base-pair binding probabilities in the wild type, whereas, the patient secondary structures showed complex folding patterns that resulted in close proximities of adjacent stem-loop structures that could potentially hinder PCBP binding events. The second binding site for PCBP was found in a bulge portion that also contained a single stem-loop structure. This site contained 5 C's in the wild type and PT5 RNA sequences, whereas in PT1 and PT6 this bulge region collapsed making the motif inaccessible to binding.

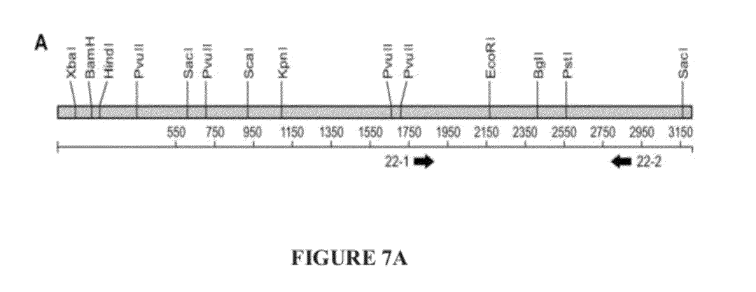

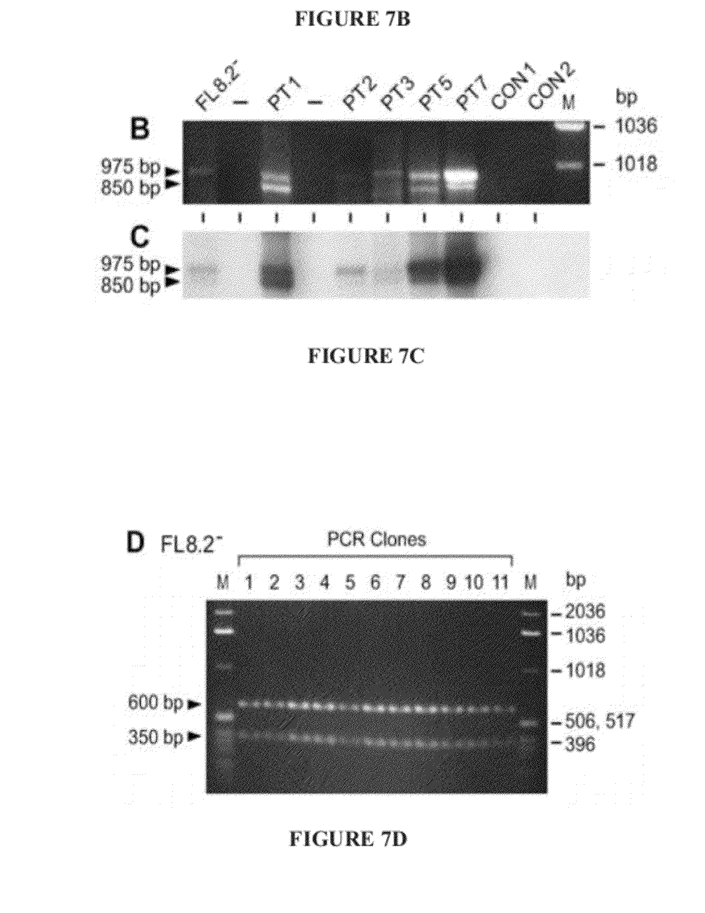

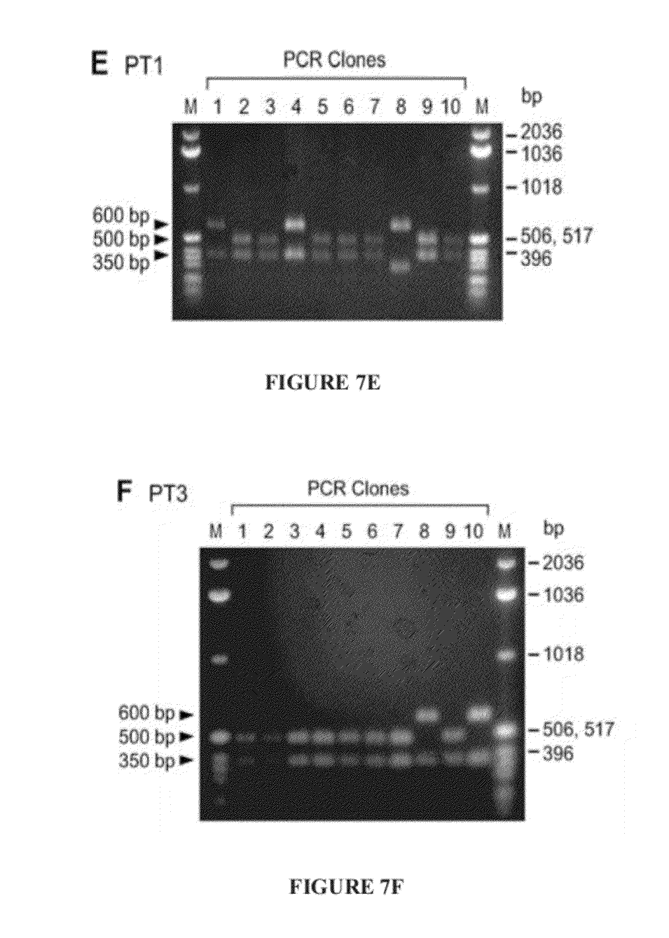

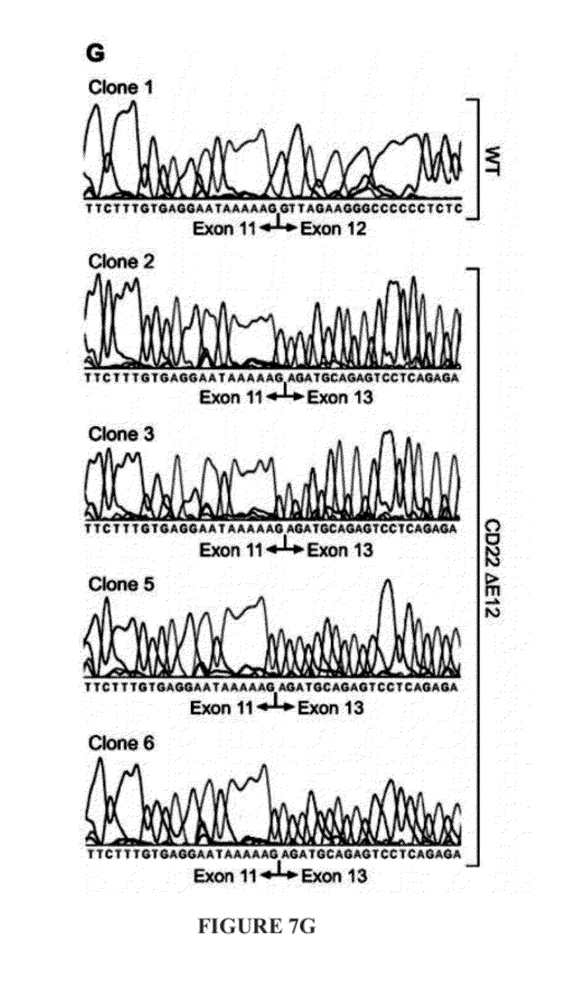

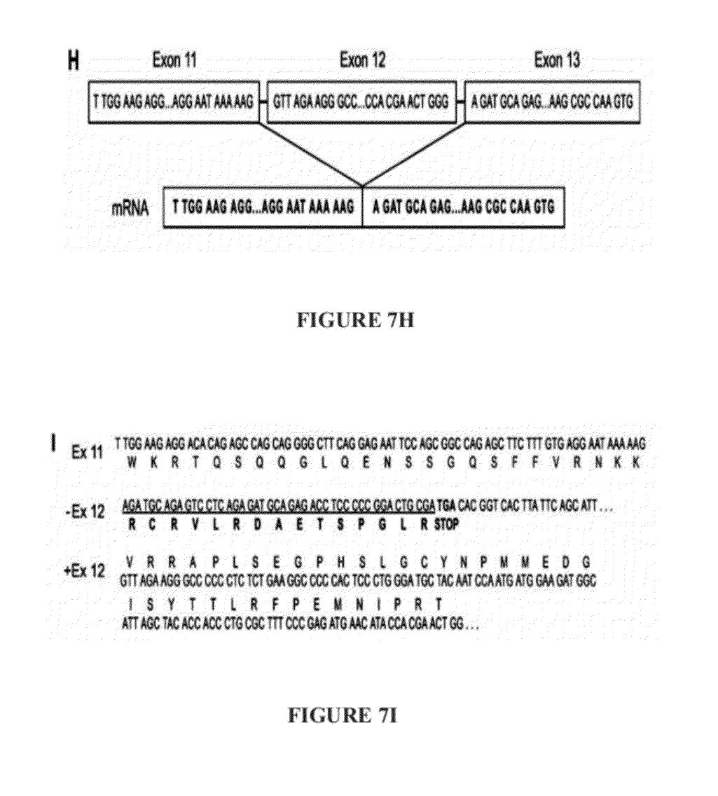

To test whether the observed CD22 mutations would affect the recognition of 5′ splice site of exon 12 by the splicing machinery and cause aberrant pre-mRNA splicing, RT-PCR assays were performed that specifically amplified a 975-bp region of CD22 mRNA (c.1801-c.2776) encompassing Exons 11-14 encoding the entire cytoplasmic domain of CD22 (FIG. 7A). RT-PCR analysis of fetal liver-derived normal B-precursor cell line FL8.2⁻ showed the anticipated 975-bp single PCR product, whereas infant BPL cells yielded a smaller second PCR product of approximately 850-bp size as well (FIG. 7B). Both PCR products hybridized to a CD22 exon 11-specific oligonucleotide probe (FIG. 7C).

EcoRI restriction analysis of cloned CD22 RT-PCR products was performed. FL8.2⁻ cells yielded two fragments of the expected sizes of 600-bp and 350-bp (FIG. 7D). In contrast, EcoRI restriction analysis of cloned CD22 RT-PCR products from primary infant BPL cells yielded abnormal fragment pairs of 500-bp (instead of 600-bp)+350-bp in the majority of the clones (FIGS. 7, E and F). These findings indicate that the truncated CD22 co-receptor in infant BPL cells is the product of abnormal CD22 mRNA species. Sequence analysis of the RT-PCR products demonstrated that the smaller approximately 850-bp RT-PCR product in infant BPL cells results from an aberrant coding sequence due to a splicing defect causing the deletion of exon 12 (c.2208-c.2327) (FIGS. 7, G and H). This exon skipping (CD22ΔE12) involves an exact splice with no other mutation at the splice junction. CD22ΔE12 was not detected in normal B-precursor cells (Table 4). A minority of PCR clones from adult hairy cell leukemia (HCL) patients and a single clone from a pediatric BPL patient also harbored CD22ΔE12 (Table 4).

TABLE 4
CD22 RT-PCR Analysis of Primary Leukemia Cells

| RNA Source | PCR Clones with CD22ΔE12 |
|---|---|
| B-lineage Leukemia Patients | |
| PT1, Infant BPL | 7/10 |
| PT3, Infant BPL | 8/10 |
| PT5, Infant BPL | 4/7 |
| PT6, Infant BPL | 2/4 |
| PT7, Pediatric BPL | 0/3 |
| PT8, Pediatric BPL | 1/4 |
| PT9, Pediatric BPL | 0/4 |
| PT10, Adult HCL | 2/21 |
| PT11, Adult HCL | 2/13 |
| PT12, Adult HCL | 2/15 |
| Normal B-Precursor Cell Lines | |
| FL8.2⁺, Fetal Liver Pro-B/T | 0/9 |
| FL8.2⁻, Fetal Liver Pro-B | 0/11 |

Without being bound by theory, it is proposed that the mutations within the downstream intronic sequence flanking exon 12 of CD22 gene contribute to the observed splicing defect in infant BPL cells by altering the genomic sequence environment for the exon 12 splice sites and influencing their recognition by the pre-mRNA splicing machinery. The observation that some infant BPL cases had very low levels of CD22 protein expression also suggests that these mutations may adversely affect pre-mRNA stability and efficiency of transcription in some cases.

The deletion of exon 12 in CD22 mRNA results in a truncating frameshift mutation starting at K736 with an insertion of 15 amino acids (RCRVLRDAETSPGLR; SEQ ID NO:3) not seen in wild-type CD22 sequence followed by a TGA termination codon (FIG. 7I). Wild-type CD22 has 14 exons; exons 3-9 each encode a single Ig domain, exon 10 encodes the transmembrane domain, whereas the cytoplasmic tail is encoded by exons 11-14 (Wilson et al. (1991), J. Exp. Med. 173, 137-146; Wilson et al. (1993), J. Immunol. 150, 5013-5024). CD22ΔE12 protein lacks the conserved tyrosines and tyrosine-based inhibitory motifs (ITIMs) that provide docking sites for the SH2 domains of the tyrosine phosphatase SHP1 (Songyang et al. (1993), Cell 72: 767-78; Law C L et al. (1996), J Exp Med 183: 547-60; Tuscano, et al. (1996), Eur. J. Immunol. 26: 1246-52; Cornall et al. (1998), Immunity 8: 497-508; Blasioli et al. (1999), J. Biol. Chem. 274: 2303-2307). It also lacks regions homologous to ITAMs (tyrosine-based activation motifs) which are docking sites for SH2 containing proteins (Songyang et al. (1993), Cell 72: 767-78; Law C L et al. (1996), J Exp Med 183: 547-60; Tuscano, et al. (1996), Eur. J. Immunol. 26: 1246-52; Cornall et al. (1998), Immunity 8: 497-508; Blasioli et al. (1999), J. Biol. Chem. 274: 2303-2307.).

A YXXM motif recognized by the N-terminal SH2 domain of the p85 subunit of PI3-kinase (Songyang et al. (1993), Cell 72: 767-78; Law C L et al. (1996), J Exp Med 183: 547-60; Tuscano, et al. (1996), Eur. J. Immunol. 26: 1246-52; Cornall et al. (1998), Immunity 8: 497-508; Blasioli et al. (1999), J. Biol. Chem. 274: 2303-2307.) is also located within the deleted cytoplasmic portion of CD22ΔE12. Thus, CD22ΔE12 mRNA encodes a truncated CD22 protein lacking most of the intracellular domain including regulatory signal transduction elements and all of the cytoplasmic tyrosine residues, which is in agreement with the results of Western blot analyses and apoptosis assays of infant BPL cells (depicted in FIG. 1).

### Example 2

### Gene Expression in Cells Expressing a Truncated CD22 Protein

Methods and Materials—hCD22ΔE12 Transgene Construct

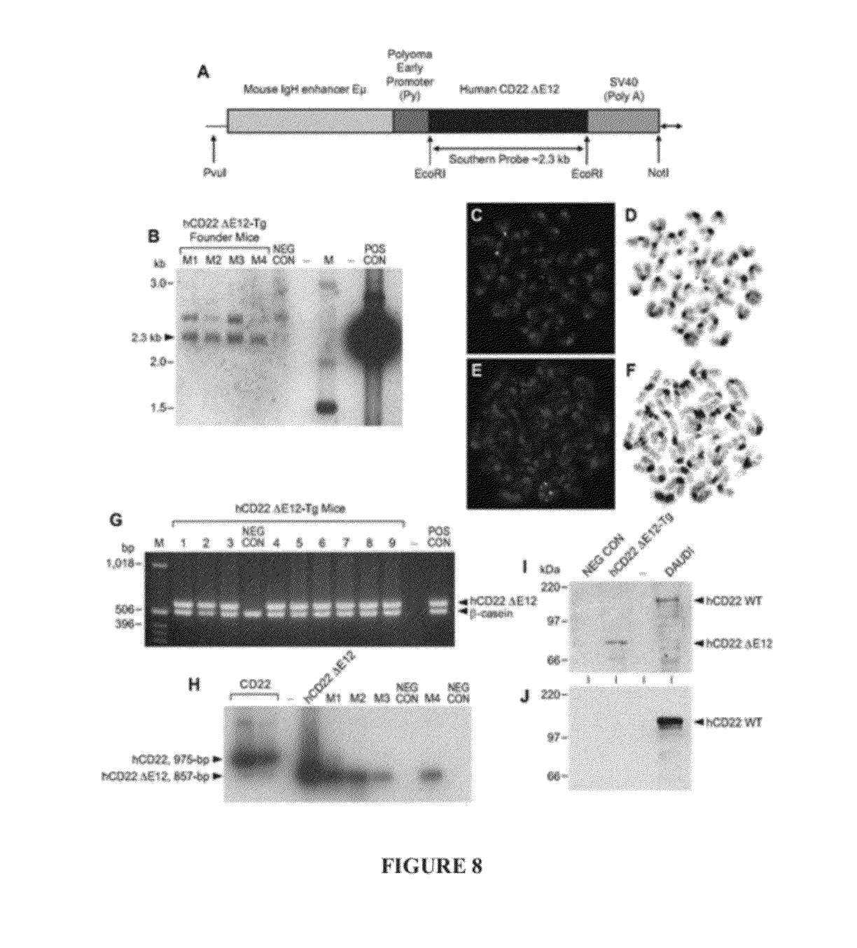

The pEμ(Py) plasmid which utilizes a polyoma early promoter regulated by immunoglobulin (Ig) heavy chain enhancer Eμ to drive B-cell specific gene expression in transgenic (Tg) mice was treated with BamHI and dephosphorylated. A 0.8-kb SV40 Poly(A) fragment was released from the pKV-461 plasmid using BamHI-BgIII. The linear pEμ(Py) and SV40 Poly(A) fragments were ligated together to create the B-lineage specific transgene cassette designated pEμ(Py)-SV40(Poly A). A full-length human CD22 cDNA with the exon 12 deletion (hCD22ΔE12) was isolated from pBluescript-CD22ΔE12 plasmid with NotI-XhoI, filled-in with Klenow polymerase, recut with PvuI, filled-in, gel-purified, and subcloned in frame between the Eμ-Py and SV40 Poly(A) sequences of the dephosphorylated SmaI-linearized pEμ(Py)-SV40 (Poly A) using standard procedures (FIG. 8 A).

### Transgenic Mice

The hCD22ΔE12 transgene construct was microinjected into the male pronucleus of fertilized FVB/N mouse oocytes using standard protocols. The oocytes were implanted into the oviducts of pseudopregnant female mice to generate hCD22ΔE12-Tg mice. Tg founder mice were identified by Southern blot analysis of EcoRI-digested genomic tail DNA using a 2.3 kb EcoRI/EcoRI [Δ-³²P]dCTP labeled CD22 probe (FIG. 8 B).

Tg founders were bred to age-matched wild-type mice to produce transgenic lines and pups were screened for the presence of the transgene by Southern blot analysis of tail-extracted DNA. Bone marrow cells from a male Tg hemizygous mouse were subjected to fluorescence in situ hybridization (FISH) analysis and reverse 4′-6-diamidino-2-phenylindole (DAPI) karyotyping to confirm the integration of the hCD22ΔE12 transgene into the mouse genome using a biotinylated human CD22 DNA FISH probe and standard procedures. Initial analysis of the mouse chromosomes indicated that the human CD22 transgene was incorporated into the mouse genome on the long arm of one chromosome 14.

A chromosome 14-specific P1-derived artificial chromosome (PAC) clone (PAC 445119, Research Genetics, Inc./Invitrogen) was used to prepare a digoxigenin-labeled FISH probe for two-color FISH analysis on the Vysis Quips system using Avidin-FITC for detection of the biotinylated human CD22 probe (green) and CY-3 (red) for detection of the digoxigenin-labeled mouse chromosome 14 probe. Both probes were hybridized together onto a slide containing the specified mouse chromosomes. Bone marrow cells displayed both probes on only one chromosome 14 with the other chromosome 14 of the diploid set displaying only the red signal consistent with the hCD22ΔE12 hemizygosity of the transgenic mouse (FIGS. 8, C and D). In another male transgenic mouse, the transgene was detected on the X-chromosome by dual labeling using the human CD22 probe and a chromosome X specific FISH probe derived from the PAC clone 473L8 (Research Genetics, Inc/Invitrogen) (FIGS. 8, E and F).

Transgenic were screened by multiplex PCR of their genomic DNA for the presence of the hCD22ΔE12 transgene and connected mIgH enhancer sequence using 5′-CCAGCCCCACCAAACCGAAAGTC-3′ (5′-primer of the mIgH enhancer; SEQ ID NO:18) and 5′-CCAGGGGCCGAGGAGATGC-3′ (3′-primer of hCD22ΔE12; SEQ ID NO:19) yielding a 0.6-kb PCR product. PCR primers for mouse β-casein exon 7, 5′GATGTGCTCCAGGCTAAAGTT-3′ (SEQ ID NO:20) and 5′-AGAAACGGAATGTTGTGGAGT-3′ (SEQ ID NO:21) provided an internal control for DNA integrity and PCR efficiency, and yielded a 0.5-kb PCR product (FIG. 8, G). The 100 μL PCR reaction consisted of 2.5 mM MgCl₂, 1×PCR buffer, 0.2 mM each deoxynucleoside triphosphate, 0.1 μM each primer, 2.5 U Taq DNA polymerase (Gibco BRL, Grand Island, N.Y.), and 2.0 μg of template DNA. The PCR conditions consisted of 31 cycles of 1 minute 15 seconds at 94° C., 2 minute 15 seconds at 60° C., and 3 minute 15 seconds at 72° C. (DeltaCycler II System, Ericomp). The PCR products were resolved on a 1×TAE agarose gel. Controls included genomic DNA from a founder mouse (POS. CON) as well as genomic DNA from a non-Tg control mouse (NEG. CON).

Tg mice were examined for expression of the hCD22ΔE12 transgene transcript in splenocytes by RT-PCR analysis using human CD22 primers spanning exon 12 (F1:5′-CCAGCCCCACCAAACCGAAAAGTC-3′ (SEQ ID NO:22); R1: 5′CCAGGGGCCGAGGAGATGC-3′ (SEQ ID NO:xx23)). Controls included the PCR products from intact human CD22 cDNA (Clontech) as well as non-Tg FVB mice. The PCR products were subjected to Southern blot analysis with an end-labeled oligonucleotide probe specific for the human CD22 Exon 11 sequence (5′-CCT GCC TCG CCA TCC TCA TCC-3′) (SEQ ID NO: 24) (FIG. 8, H). A plasmid containing hCD22ΔE12 served as a positive control for the transgene and human CD22 cDNA (Clontech) was included as an additional control for the Southern blot.

General procedures for Southern blot analysis, PCR, RT-PCR, Western blot analysis, and karyotyping were previously published (Reference 19-23). Single-cell suspensions of splenocytes and bone marrow cells obtained from electively sacrificed 6-7 wk old Tg mice and their wild-type controls were purged of erythrocytes by hypotonic lysis and immunophenotyped by direct fluorescence staining and flow cytometry using anti-CD19-phycoerythrin (PE), anti-B220/CD45R-PE, and anti-IgM-FITC, using published procedures. The PHI Animal Care and Use Committee (IACUC) approved Mouse experiments, and all animal care procedures conformed to the Guide for the Care and Use of Laboratory Animals of the National Research Council (National Academy Press, Washington D.C. 1996).

Gene Expression Profiling of Splenocytes from hCD22ΔE12-Tg Mice and Primary Leukemic Cells from Patients with Newly Diagnosed Infant vs. Pediatric ALL Expression profiling of mouse splenocytes for 588 genes in six functional groups was performed using the Atlas Mouse cDNA Expression arrays from Clontech Laboratories Inc. (Cat. Number 634539) according to the manufacturer's specification using standard procedures. Density readings from the phosphor images were normalized to 5 housekeeping genes (glyceraldehyde-3-phosphate dehydrogenase (G3PDH; GADPH), myosin I, ornithine decarboxylase (ODC), phospholipase A and hypoxantine-guanine phosphoribosyltransferase (HPRT)). The expression values of the housekeeping genes were within the dynamic range of the expression values of the genes on the array (34-233 units after background subtraction).

The average background subtracted expression values for the 5 housekeeping genes ranged from 26.2 to 45.7 across the 10 samples. The tenth percentile mean and standard deviation pixel intensity values for the genes represented on the array were considered to be ‘blank’ spots, and were used to calculate the ‘presence’ and ‘absence’ calls. Genes were considered ‘present if the mean expression value of the gene was greater than 3 standard deviations from the mean expression value of the blank spots. All expression levels were log to the base 2 transformed for statistical comparisons.

The normalization procedure was examined by performing bi-variate plots between two samples such that the expression values were equally dispersed around the line of unity and this was a necessary prerequisite to determine differentially expressed genes between hCD22ΔE12-Tg and control FVB mice. T-tests with degrees of freedom correction for unequal variances (Excel formula) were performed on normalized values to identify discriminating genes between hCD22ΔE12-Tg and FVB control mice and True Discovery Rates were calculated using observed and expected number of changes at three p-values (0.01, 0.02, 0.05).

Gene expression changes were visualized using mean centered and standardized expression values from the 10 samples represented on a cluster figure. We employed a one-way agglomerative hierarchical clustering technique to organize expression patterns using the average distance linkage method. For enrichment analysis, we utilized a publically available web tool to identify over-represented functional annotations using a curated, standardized set of description terms (http://amigo.geneontology.org/cgi-bin/amigo/term enrichment). Gene ontology term for ‘GO:0048523 negative regulation of cellular process’ constituted 178 genes on the Clontech Mouse array of which a significant proportion (23 genes compared to 25 genes/410 genes unaffected, Fishers Exact Test, 2-tailed, p=0.008) were differentially regulated in hCD22ΔE12-Tg mice.

The transgenic mouse gene expression profile was deposited with GEO (ncbi.nlm.nih.gov/geo/) confidentially and will be publically available on Sep. 13, 2010 as GEO Accession Number GSE23998.

### Human Pediatric Patients

Expression profiling of primary leukemia cells from 31 infants and 30 non-infant pediatric patients with newly diagnosed ALL for 588 genes in six functional groups was performed using the Atlas human cDNA expression arrays from Clontech Laboratories Inc. (Cat No 634511) according to the manufacturer's specification using previously detailed standard procedures and described above for the mouse array. Pixel processing of the digital images was also performed using the procedure outlined for the mouse array. To compare the gene expression levels from the ALL patients, a normalization procedure was applied using the raw signal intensities. In brief, the mean signal intensity within each spot was subtracted from the sub-grid median of the background signal. For thresholding, the intensities of the duplicate spots for each gene were averaged and floored to a signal value of 2 units. Density readings from the phosphorimages were normalized to 5 housekeeping genes (glyceraldehyde 3-phosphate dehydrogenase (GAPDH); brain-specific tubulin alpha 1 subunit (TUBA1); HLA class I histocompatibility antigen C-4 alpha subunit (HLAC); cytoplasmic beta-actin (ACTB); ubiquitin). All expression levels were log to the base 2 transformed. T-tests with degrees of freedom correction for unequal variances (Excel formula) were performed on normalized values to identify discriminating genes between patient subsets.

The gene expression profile of the 61 pediatric patients was deposited confidentially with GEO (www.ncbi.nlm.nih.gov/geo/) and will be publically available on Sep. 13, 2010 under GEO Accession Number GSE24000.

To visualize the gene expression relationships between genes across samples for both hCD22ΔE12-Tg mice and newly diagnosed leukemia patients, a one-way agglomerative hierarchical clustering technique was performed to organize expression patterns using the average distance linkage method using mean centered, standardized intensity values after log 2 transformation and normalization procedure outlined above. Most consistent discriminating genes in both the human and the mouse cDNA expression arrays were cross-referenced to the Oncomine™ Research Data Base (http://www.oncomine.org/) for leukemia and lymphoma studies.

A meta-analysis was used to interrogate each of the signature genes for its previously reported expression values and associations in other B-lineage leukemia (10 studies; 11 comparisons) or lymphoma studies (5 studies; 15 comparisons) in the Oncomine database. For each gene the fold difference and T-test p-value are reported for log-transformed, normalized expression levels. In order to control for normalization artifacts in the evaluation of significant differences between this study and other published studies, gene expression profiles were compared using the same set of housekeeping genes. Specifically, the distribution of the average fold-difference values for the five housekeeping genes were examined from the leukemia and lymphoma studies reported on the Oncomine database for outliers that affected the comparison with this study (GAPDH, HPRT1, ODC, PLA2G1B, MYH6 representing the human orthologs of the housekeeping genes used for normalization on the mouse array and showed expression values within the dynamic range of the measured pixel intensities). Mouse ortholog genes significantly down-regulated in hCD22ΔE12-Tg mice were analyzed for groups of genes assigned as cyclins, CDK inhibitors, tumor suppressors and G-proteins as assigned by Clontech. Fishers exact test (2-tailed, P≦0.05 deemed significant) was performed comparing the proportion of significantly assigned changes using the housekeeping genes and those differentially expressed in both hCD22ΔE12-Tg mice and infant ALL patients.

### Results

To further examine the functional effect of the exon 12 splicing defect on B-lineage lymphoid cells, transgenic mice were produced with human CD22ΔE12 under control of the immunoglobulin enhancer Eμ that is activated in early B-cell ontogeny prior to Ig gene rearrangements (FIG. 8). Western blot analysis of splenocytes from hCD22ΔE12-Tg mice (but not transgene negative control mice) using N-20, a polyclonal anti-CD22 antibody recognizing the N-terminus of the human CD22 molecule (Santa Cruz, Catalog #7031) revealed the presence of a truncated CD22, which was not reactive with C-20, a C-terminal anti-CD22 antibody (Santa Cruz, Catalog #7029), reminiscent of the Western blot results obtained with human infant leukemia cells (FIGS. 8, I and J).

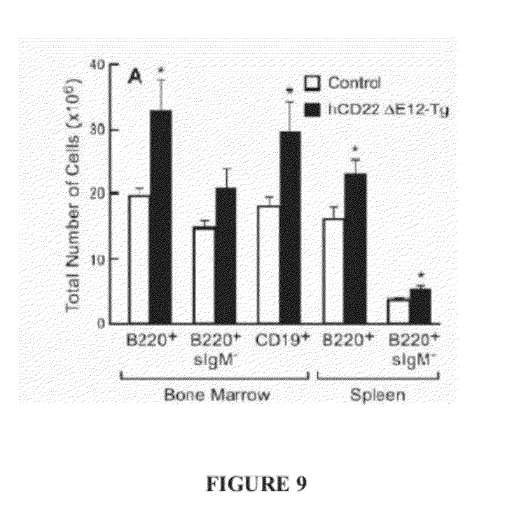

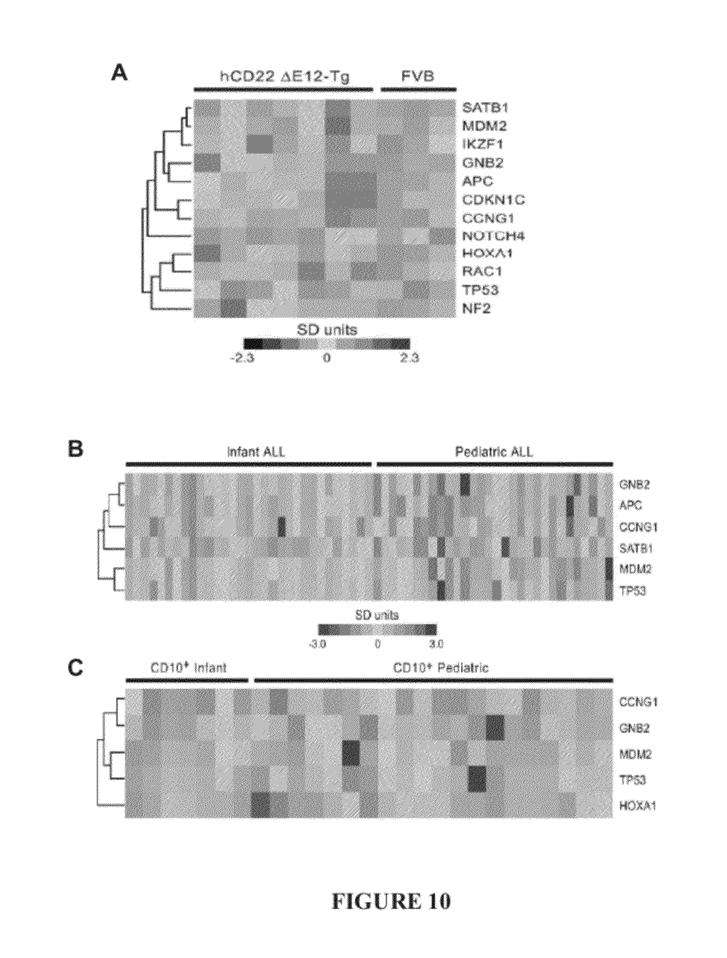

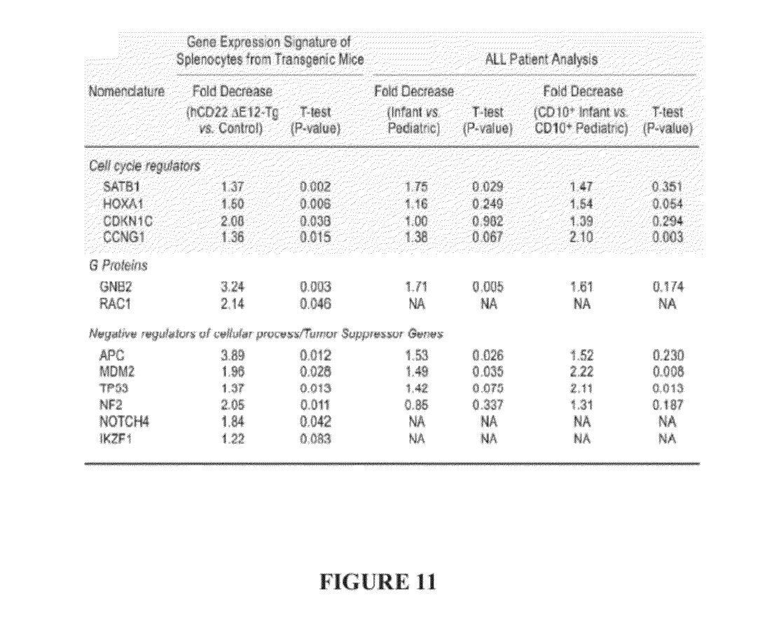

At 6 weeks of age, hCD22ΔE12 transgenic mice showed flow cytometric evidence for B-precursor/B-cell hyperplasia (FIG. 9A). The B220⁺ (23.0±2.2×10⁶/spleen vs. 16.2±1.8×10⁶/spleen, T-test P=0.042; 32.9±4.7×10⁶/bone marrow from 2 femurs vs. 19.9±0.9×10⁶/bone marrow from 2 femurs, T-test P-value=0.026) total B-lineage lymphoid cell numbers as well as B220⁺sIgM⁻ B-precursor numbers (5.3±0.5×10⁶/spleen vs. 3.6±0.3×10⁶/spleen, T-test P-value=0.017; 20.8±3.1×10⁶/bone marrow from 2 femurs vs. 15.0±0.8×10⁶/bone marrow from 2 femurs, T-test P-value=0.1) in the spleen as well as bone marrow were moderately elevated in hCD22ΔE12 transgenic mice. Likewise, there were more CD19⁺ B-lineage lymphoid cells in the bone marrows of hCD22ΔE12-Tg mice than in control mice (29.7±4.5×10⁶ vs. 18.0±1.4×10⁶, P=0.038).

To examine the deregulatory biologic effects of the expression of the defective CD22ΔE12 protein at a molecular level, the gene expression profiles of splenocytes from hCD22ΔE12 transgenic mice were compared to non-transgenic wild-type control mice. Twelve differentially expressed genes that had standardized values of expression outside the range of the control values in wild-type mice were classified as the most discriminating genes. This CD22ΔE12-associated 12-gene signature transcriptome included (i) tumor suppressor genes TP53 (as well as TP53 regulator MDM2), neurofibromatosis 2 (NF2) (as well as NF2 regulator RAC1), and the adenomatous polyposis coli (APC) gene, a tumor suppressor known to regulate the Wnt/beta-catenin signaling, (ii) genes for chromatin remodeling/global gene expression regulators with a tumor suppressor function IKZF1/IKAROS and SATB1, as well as (iii) cell cycle regulatory genes CDKN1C, CCNG1, and NOTCH4 (FIG. 9, B). These results provide evidence that CD22ΔE12 alters the regulation of gene expression and results in reduced expression levels of several genes that have a tumor suppressor function.

Gene expression profiling of primary leukemia cells from 31 infants and 30 non-infant children with ALL was performed to determine if any of the CD22ΔE12-associated signature genes identified in mice are differentially expressed in infant ALL vs. pediatric ALL. Reduced expression levels of 6 of the 9 CD22ΔE12 signature genes that were represented on the human cDNA arrays, including TP53 and APC as well as MDM2, SATB1, CCNG1 and GNB2 discriminated infant BPL from non-infant BPL (FIG. 9, B). The signature transcriptome was confirmed as independent of MLL gene rearrangements by comparing the gene expression profiles of CD10 antigen positive infant leukemia cells that do not have MLL gene rearrangements with those of CD10⁺ pediatric ALL cells (FIG. 9, B). The data suggest that CD22ΔE12 directly contributes to the biology of infant leukemia.

A meta-analysis was used to interrogate each of the most discriminating signature genes with significant T-test statistics (viz., APC, GNB2, MDM2, and SATB1) for its previously reported expression values and associations in 10 B-lineage leukemia studies with 11 comparative analyses and 5 B-lineage lymphoma studies with 15 comparative analyses in the Oncomine™ Research Data Base (Rhodes et al. (2007), Neoplasia 9:166-180). Each of these genes was expressed in malignant cells from patients with B-lineage lymphoid malignancies at significantly lower levels than in normal B-cell controls (Table 5).

TABLE 5
Meta-Analysis of CD22ΔE12 Signature Gene Expression -
Oncomine ™ Database

| | Ref. | | | | |
|---|---|---|---|---|---|
| Comparison (versus Normal) | No. | APC | GNB2 | MDM2 | SATB1 |
| A. B-lineage Leukemias | | | | | |
| BPL | 2 | −3.75 | −1.55 | | |
| BPL | 5 | −1.27 | | | |
| CLL | 1 | −1.33 | | −1.44 | −3.11 |
| CLL | 3 | | | | −3.95 |
| CLL | 4 | | | −1.55 | −2.79 |
| CLL | 6 | | | −1.86 | −5.04 |
| HCL | 3 | −1.35 | | −1.87 | −2.65 |
| B. B-lineage Lymphomas | | | | | |
| Diffuse Large B-Cell | 1 | −1.59 | | | −2.22 |
| Lymphoma | | | | | |
| Burkitt's Lymphoma | 3 | −1.54 | | | −6.23 |
| Centroblastic Lymphoma | 3 | −1.87 | | | −3.56 |
| Cutaneous Follicular | 8 | −1.38 | | −1.44 | |
| Lymphoma | | | | | |
| Diffuse Large B-Cell | 1 | −1.61 | | | −2.61 |
| Lymphoma | | | | | |
| Diffuse Large B-Cell | 3 | −1.48 | | | −2.90 |
| Lymphoma | | | | | |
| Diffuse Large B-Cell | 7 | | | −1.39 | |
| Lymphoma | | | | | |
| Diffuse Large B-Cell | 6 | | | −1.42 | −3.65 |
| Lymphoma | | | | | |
| Diffuse Large B-Cell | 8 | −1.61 | | | |
| Lymphoma | | | | | |
| Follicular Lymphoma | 1 | | | −1.26 | −1.57 |
| Follicular Lymphoma | 3 | −1.32 | | | |
| Follicular Lymphoma | 6 | | | −2.01 | −2.83 |
| Germinal Center B-Cell-Like, | 1 | −1.58 | | | −2.26 |
| Diffuse Large B-Cell | | | | | |
| Lymphoma | | | | | |
| Mantle Cell Lymphoma | 3 | | | | −5.86 |
| Marginal Zone B-Cell | 8 | −1.73 | | −1.36 | |
| Lymphoma | | | | | |
| Comparison (versus Normal) | | | | | |

Four signature genes that were significantly down-regulated in Infant ALL patients and hCD22ΔE12-Tg mice were interrogated using the Oncomine database for their expression in other studies comparing B-lineage leukemias and non-Hodgkin's lymphomas. (A) Enrichment of the gene expression signature was observed in 7 out of the 11 comparisons for three different B-lineage leukemias deposited into the database. Fold differences relative to ‘normal’ B-cell/B-precursor expression (T-test p-values<0.05, negative values represent down-regulation in leukemic cells) are shown for BPL, CLL, and HCL cells. (B) Enrichment of the gene expression signature was observed in 15 out of the 18 comparisons for 9 different B-lineage lymphomas deposited into the Oncomine database. Fold differences relative to ‘normal’ expression (T-test P-values<0.05, negative values represent down regulation in leukemic cells) are shown for comparisons of B-lineage lymphoma cells with normal B-cells.

The invention being thus described, it will be apparent to one of ordinary skill in the art that various modifications of the materials and methods for practicing the invention can be made. Such modifications are to be considered within the scope of the invention as defined by the following claims.

Each of the references from the patent and periodical literature cited herein is hereby expressly incorporated in its entirety by such citation.

1. An isolated polynucleotide molecule encoding a mutated CD22 polypeptide having all or a part of Exon 12 deleted, or a fragment of said isolated polynucleotide.

2. The isolated polynucleotide molecule of claim 1, wherein said molecule is a probe that hybridizes with and/or is used to amplify all or a portion of a polynucleotide encoding a CD22ΔE12 mutant polypeptide.

3. A method of identifying the presence of the polynucleotide of claim 1 in a sample, comprising analyzing the sample for the presence of a CD 22 polynucleotide sequence mutation that alters splicing of exon 12.

4. The method of claim 3, wherein said identification includes single-nucleotide polymorphism analysis, PCR, RT-PCR, or a combination of these.

5. The method of claim 3, wherein said identification further includes sequence analysis.

6. An isolated antisense oligonucleotide having a nucleotide sequence that selectively binds the polynucleotide of claim 1 at Exon 12 to inhibit exclusion of exon 12 during splicing of CD22 mRNA.

7. The antisense oligonucleotide of claim 6, wherein the oligonucleotide is a morpholino.

8. An isolated polypeptide encoded by the polynucleotide of claim 1.

9. The isolated polypeptide of claim 8, having an amino acid sequence comprising: RCRVLRDAETSPGLR.

10. An isolated antibody that specifically binds the polypeptide of claim 9 at the amino acid sequence RCRVLRDAETSPGLR.

11. The antibody of claim 10, further comprising a toxin.

12. A method for identifying if a subject's B-cell disorder is likely to be susceptible or resistant to treatment with an anti-CD22 antibody, said method comprising:
a) analyzing a leukemic cell sample obtained from the subject for expression of the isolated CD22ΔE12 polynucleotide of claim 1; and
b) identifying the subject's disorder as likely to be resistant to said treatment and not treating with anti-CD22 antibody if the subject's sample expresses the CD22ΔE12 polynucleotide of claim 1; or
c) identifying the subject's disorder as likely to be susceptible to said treatment and treating with anti-CD22 antibody if the subject's sample does not to express the CD22ΔE12 polynucleotide of claim 1.

13. A method of treating a subject suffering from a B-cell disorder, said method comprising:
treating said subject with an anti-CD22 antibody if said subject's leukemia cell sample expresses the CD22ΔE12 polynucleotide of claim 1.

14. A method of treating a subject suffering from a B-cell disorder, said method comprising:
treating said subject with the antisense oligonucleotide of claim 6 if said subject's leukemia cell sample expresses the CD22ΔE12 polynucleotide of claim 1.

15. (canceled)

16. A method for treating a B-cell disorder in a subject, the method comprising:
a) analyzing a sample obtained from the subject for the presence of leukemic cells expressing the CD22ΔE12 polynucleotide of claim 1; and
b) identifying and treating the subject's B-cell disorder as an aggressive B-cell disorder, if the subject's sample is found to express the CD22ΔE12 polynucleotide of claim 1.

17. A method of treating a subject as having an increased risk for developing leukemia comprising:
a) analyzing a sample obtained from the subject for the presence of genomic CD22 intronic mutations in heterozygous or homozygous constellation that induce expression of the CD22ΔE12 polynucleotide of claim 1; and
b) identifying the subject as having increased risk for developing leukemia if the subject's sample is found to contain the genomic CD22 intronic mutations.

18. A method of identifying parents at risk of having children with leukemia, comprising:
a) analyzing a sample obtained from the parents for the presence of genomic CD22 intronic mutations in heterozygous or homozygous constellation that induce expression of the CD22ΔE12 polynucleotide of claim 1; and
b) identifying the parents as at risk of having children with leukemia if the subject's sample is found to contain the genomic CD22 intronic mutations.

19. A method of treating a subject to reduce or eliminate expression of the CD22ΔE12 polynucleotide of claim 1, comprising:
administering to the subject an interfering RNA directed to reduce or eliminate the Exon 12 deletion mutant expressed in the subject.

20. A method of treating a subject suffering from leukemia or lymphoma comprising administering to the subject a composition comprising oligonucleotides dispersed within nanoparticles, the oligonucleotides having a sequence designed to bind the CD22ΔE12 polynucleotide of claim 1.

21. A method of treating a subject suffering from leukemia or lymphoma comprising administering to the subject a composition comprising an antibody that specifically binds a polypeptide or fragment expressed by the polynucleotide of claim 1.

22. A method of claim 20, where the polypeptide comprises the sequence of SEQ ID NO:3.
